(19) United States
(12) Patent Application Publication
(10) Pub. No.: US 2019/0330337 A1
(43) Pub. Date: Oct. 31, 2019

(54) Tunable Chimeric Antigen Receptors

(71) Applicant: AUTOLUS LIMITED, London (GB)

(72) Inventors: Martin Pulé, London (GB); Simon Thomas, London (GB); Shaun Cordoba, London (GB); Evangelia Kokalaki, London (GB)

(21) Appl. No.: 16/310,076

(22) PCT Filed: Jun. 15, 2017
(86) PCT No.: PCT/GB2017/051743

§ 371 Date: Dec. 14, 2018

(30) Foreign Application Priority Data
Jun. 16, 2016 (GB) 1610515.7

(51) Int. Cl. C07K 16/28; C12N 5/0783; A61K 35/17; C07K 14/705; C07K 14/725

(52) U.S. Cl. CPC C07K 16/2803; A61K 38/00; A61K 35/17; C07K 14/70521; C07K 14/7051; C07K 14/70517; C07K 14/70578; C07K 2319/02; C07K 2319/33; C07K 2319/03; C07K 2317/622; C07K 2317/24; C07K 2317/53; C07K 2317/524; C07K 2317/526; C07K 2317/92; C12N 5/0636

(57) ABSTRACT

The present invention provides a cell which co-expresses a first chimeric antigen receptor (CAR) and second CAR at the cell surface, each CAR comprising an antigen-binding domain, a transmembrane domain and an intracellular domain wherein the antigen-binding domain of the first CAR binds to CD19 and the antigen-binding domain of the second CAR binds to CD22; and wherein the first and/or second CAR is a tunable CAR having an intracellular domain comprising a heterodimenzation domain, which intracellular domain is capable of binding a separate intracellular signalling molecule which comprises a reciprocal heterodimenzation domain and a signalling domain.

### FIELD OF THE INVENTION

The present invention relates to a cell which comprises more than one chimeric antigen receptor (CAR).

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

Chimeric Antigen Receptors (CARs)

Chimeric antigen receptors are proteins which graft the specificity of, for example, a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

CD19

The human CD19 antigen is a 95 kd transmembrane glycoprotein belonging to the immunoglobulin superfamily. CD19 is expressed very early in B-cell differentiation and is only lost at terminal B-cell differentiation into plasma cells. Consequently, CD19 is expressed on all B-cell malignancies apart from multiple myeloma. Since loss of the normal B-cell compartment is an acceptable toxicity, CD19 is an attractive CAR target and clinical studies targeting CD19 with CARs have seen promising results.

However it has been observed that using a CD19 CAR approach for cancer treatment, tumour heterogeneity and immunoediting can cause escape from CAR treatment. For example, in the study described by Grupp et al (2013; New Eng. J. Med 368:1509-1518, paper No 380, ASH 2014) CAR-modified T cell approach was used for the treatment of acute B-lymphocytic leukemia. In that clinical trial it was found that 10 patients with a complete remission after one month did relapse and 5 of them relapsed with CD19-negative disease.

There is thus a need for alternative CD19 CAR treatment approaches which address the problems of cancer escape and tumour heterogeneity.

Another problem associated with CD19 CAR treatment is toxicity. In a CD19 CAR clinical trial treating adults with B cell acute lymphoblastic leukaemia, 25 out of the 30 patients developed cytokine release syndrome (CRS) after CAR-T cell infusion, and seven developed severe CRS requiring admission to an intensive care unit (Turtle et al 2016 J. Clin Invest. http://dx.doi.org/10.1172/JC185309). CRS is initiated by activation and proliferation of CAR-T cells after recognition of CD19+ target cells and is characterised by elevated serum levels of IL-6 and IFN-γ. Severe neurotoxicity also occurred in 15 of the 30 patients. The clinical presentation of neurotoxicity was variable and included mild to severe encephalopathy, focal neurologic deficits, and in 3 patients, generalized seizures.

In relapsed and refractory chronic lymphocytic leukaemia (CLL) a study with 14 patients with CD19 CAR-T cells gave a response rate of 57% with 4 complete remissions. However all patients developed B cell aplasia and experiences CRS (Porter et al (2015) Science Translational Medicine 7: 303 303ra139).

There is thus a need for alternative CD19 CAR treatment approaches which address the problem of toxicities associated with CD19 CAR-T cell treatment.

### SUMMARY OF THE INVENTION

The present inventors have developed a CAR T cell which expresses two CARs at the cell surface, one specific for CD19 and one specific for CD22. Signalling via the CD19 and/or CD22 CAR is/are controllable using an agent, such as a small molecule, which disrupts the CAR signalling system.

Thus in a first aspect the present invention provides a cell which co-expresses a first chimeric antigen receptor (CAR) and second CAR at the cell surface, each CAR comprising an antigen-binding domain, a transmembrane domain and an intracellular domain
wherein the antigen-binding domain of the first CAR binds to CD19 and the antigen-binding domain of the second CAR binds to CD22; and
wherein the first and/or second CAR is a tunable CAR having an intracellular domain comprising a heterodimerization domain, which intracellular domain is capable of binding a separate intracellular signalling molecule which comprises a reciprocal heterodimerization domain and a signalling domain.

Binding of the first and second/or CAR to the intracellular signalling molecule may be disrupted by the presence of an agent, such that in the absence of the agent the first and/or second CAR heterodimerize(s) with the intracellular signalling molecule and binding of the antigen binding domain to antigen results in signalling through the signalling domain; whereas in the presence of the agent, the first and/or second CAR do/does not heterodimerize with the intracellular signalling molecule and binding of the antigen binding domain to antigen does not result in signalling through the signalling domain.

There is thus provided a cell which expresses a CAR signalling system comprising:

(i) a receptor component (the tunable CAR) comprising an antigen binding domain, a transmembrane domain and a first binding domain; and
(ii) an intracellular signalling component comprising a signalling domain and a second binding domain which specifically binds the first binding domain of the receptor component;

wherein, binding of the first and second binding domains is disrupted by the presence of an agent, such that in the absence of the agent the receptor component and the signalling component heterodimerize and binding of the antigen binding domain to antigen results in signalling through the signalling domain, whereas in the presence of the agent the receptor component and the signalling component do not heterodimerize and binding of the antigen binding domain to antigen does not result in signalling through the signalling domain.

The CD19 CAR may be tunable and part of a CAR signalling system as defined above. The CD22 CAR may be a “classical” CAR which comprises an endodomain integral to the molecule.

The fact the one CAR binds CD19 and the other CAR binds CD22 is advantageous because some lymphomas and leukaemias become CD19 negative after CD19 targeting, (or possibly CD22 negative after CD22 targeting), so it gives a “back-up” antigen, should this occur.

The cell may be an immune effector cell, such as a T-cell or natural killer (NK) cell. Features mentioned herein in connection with a T cell apply equally to other immune effector cells, such as NK cells.

Each CAR may comprise:
(i) an antigen-binding domain;
(ii) a spacer; and
(iii) a trans-membrane domain.

The spacer of the first CAR may be different to the spacer of the second CAR, such the first and second CAR do not form heterodimers.

The spacer of the first CAR may have a different length and/or configuration from the spacer of the second CAR, such that each CAR is tailored for recognition of its respective target antigen.

The antigen-binding domain of the second CAR may bind to a membrane-distal epitope on CD22. The antigen-binding domain of the second CAR may bind to an epitope on Ig domain 1, 2, 3 or 4 of CD22, for example on Ig domain 3 of CD22.

The antigen-binding domain of the first CAR may bind to an epitope on CD19 which is encoded by exon 1, 3 or 4.

The first CAR, which binds to CD19, may be a tunable CAR, having an intracellular domain which comprises a heterodimerization domain which binds a heterodimerization domain of an intracellular signalling molecule; and the second CAR, which binds to CD22, may be a classical CAR, having an intracellular domain which comprises a signalling domain.

Alternatively, the first CAR, which binds to CD19, may be a classical CAR, having an intracellular domain which comprises a signalling domain; and the second CAR, which binds to CD22, may be a tunable CAR, having an intracellular domain which comprises a heterodimerization domain which binds a heterodimerization domain of an intracellular signalling molecule.

Alternatively both the first and second CAR may be tunable CARs, each having an intracellular domain which comprises a heterodimerization domain which binds a heterodimerization domain of an intracellular signalling molecule.

In this embodiment where both CARs comprise an intracellular domain which comprises a heterodimerization domain which binds a heterodimerization domain of an intracellular signalling molecule, the first and second CAR independently may bind the same intracellular signalling molecule. Both binding of the first CAR to the intracellular signalling molecule; and binding of the second CAR to the intracellular signalling molecule may be disrupted by the presence of the same agent.

Alternatively, the first CAR may bind to a first intracellular signalling molecule and the second CAR may bind to a second, distinct, signalling molecule. In this respect, binding of the first CAR to the first intracellular signalling molecule may be disrupted by the presence of a first agent; and binding of the second CAR to the second intracellular signalling molecule may be disrupted by the presence of a second agent.

The antigen-binding domain of the first CAR may comprise

a) a heavy chain variable region (VH) having complementarity determining regions (CDRs) with the following sequences:

(SEQ ID No. 1)

CDR1 - SYWMN;

(SEQ ID No. 2)

CDR2 - QIWPGDGDTNYNGKFK

(SEQ ID No. 3)

CDR3 - RETTTVGRYYYAMDY;

and
b) a light chain variable region (VL) having CDRs with the following sequences:

(SEQ ID No. 4)

CDR1 - KASQSVDYDGDSYLN;

(SEQ ID No. 5)

CDR2 - DASNLVS

(SEQ ID No. 6)

CDR3 - QQSTEDPWT.

The antigen binding domain of the first CAR may comprise a VH domain having the sequence shown as SEQ ID No. 7, or SEQ ID NO 8; or a VL domain having the sequence shown as SEQ ID No 9, SEQ ID No. 10 or SEQ ID No. 11 a variant thereof having at least 90% sequence identity which retains the capacity to bind CD19.

The antigen binding domain of the first CAR may comprise the sequence shown as SEQ ID No. 12, SEQ ID No. 13 or SEQ ID No. 14 or a variant thereof having at least 90% sequence identity which retains the capacity to bind CD19.

The antigen-binding domain of the second CAR may comprise

a) a heavy chain variable region (VH) having complementarity determining regions (CDRs) with the following sequences:

(SEQ ID No. 15)

CDR1 - NYWIN;

(SEQ ID No. 16)

CDR2 - NIYPSDSFTNYNQKFKD

(SEQ ID No. 17)

CDR3 - DTQERSWYFDV;

and
b) a light chain variable region (VL) having CDRs with the following sequences:

(SEQ ID No. 18)

CDR1 - RSSQSLVHSNGNTYLH;

(SEQ ID No. 19)

CDR2 - KVSNRFS

(SEQ ID No. 20)

CDR3 - SQSTHVPWT.

The antigen binding domain of the second CAR may comprise a VH domain having the sequence shown as SEQ ID No. 21, or SEQ ID NO 22; or a VL domain having the sequence shown as SEQ ID No 23, or SEQ ID No. 24 or a variant thereof having at least 90% sequence identity which retains the capacity to bind CD22.

The antigen binding domain of the second CAR may comprise the sequence shown as SEQ ID No 25 or SEQ ID No. 26 or a variant thereof having at least 90% sequence identity which retains the capacity to bind CD22.

The first and/or second CAR may comprise a coiled-coil spacer domain. In particular the second CAR may comprise a coiled-coil spacer domain.

The coiled-coil spacer domain may enables the multimerization of at least three CAR-forming polypeptides. For example, the CAR may be made up of five CAR-forming polypeptides, giving a pentameric CAR.

The coiled-coil spacer domain may be from, for example: cartilage-oligomeric matrix protein (COMP), mannose-binding protein A, coiled-coil serine-rich protein 1, polypeptide release factor 2, SNAP-25, SNARE, Lac repressor or apolipoprotein E.

The coiled-coil spacer domain may comprise one of the sequences shown as SEQ ID No. 50 to 63 or a fragment thereof or a variant thereof which has at least 80% sequence identity.

The cell may also comprise one or more intracellular signalling molecule(s) as defined above.

In a second aspect, the present invention provides a nucleic acid construct encoding both the first and second chimeric antigen receptors (CARs) as defined above.

The nucleic acid construct may have one of the following structures:

a) AgB1-spacer1-TM1-HD1-coexpr-AgB2-spacer2-TM2-endo2;
b) AgB1-spacer1-TM1-endo1-coexpr-AgB2-spacer2-TM2-HD2;
c) AgB2-spacer2-TM2-HD2-coexpr-AgB1-spacer1-TM1-endo1;
d) AgB2-spacer2-TM2-endo2-coexpr-AgB1-spacer1-TM1-HD1;
e) AgB1-spacer1-TM1-HD1-coexpr-AgB2-spacer2-TM1-HD1;
f) AgB2-spacer2-TM2-HD2-coexpr-AgB1-spacer1-TM1-HD1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR
HD1 is a nucleic acid sequence encoding a heterodimerisation domain of the first CAR
Endo1 is a nucleic acid sequence encoding an intracellular domain which comprises a signalling domain of the first CAR;
coexpr is a nucleic acid sequence enabling co-expression of both CARs
AgB2 is a nucleic acid sequence encoding the antigen-binding domain of the second CAR;
spacer 2 is a nucleic acid sequence encoding the spacer of the second CAR;
TM2 is a a nucleic acid sequence encoding the transmembrane domain of the second CAR;
HD2 is a nucleic acid sequence encoding a heterodimerisation domain of the second CAR
Endo2 is a nucleic acid sequence encoding an intracellular domain which comprises a signalling domain of the second CAR.

The nucleic acid sequence coexpr may encode a sequence comprising a self-cleaving peptide.

Alternative codons may be used in regions of sequence encoding the same or similar amino acid sequences, in order to avoid homologous recombination.

The nucleic acid construct may also comprise a nucleic acid sequence encoding an intracellular signalling molecule as defined above.

In a third aspect, the present invention provides a kit which comprises
(i) a first nucleic acid sequence encoding the first chimeric antigen receptor (CAR) as defined above, which nucleic acid sequence has one of the following structures:
   AgB1-spacer1-TM1-HD1 or AgB1-spacer1-TM1-endo1
   in which
   AgB1 is a nucleic acid sequence encoding the antigen-binding domain of the first CAR;
   spacer 1 is a nucleic acid sequence encoding the spacer of the first CAR;
   TM1 is a nucleic acid sequence encoding the transmembrane domain of the first CAR;
   HD1 is a nucleic acid sequence encoding a heterodimerisation domain of the first CAR; and
   Endo1 is a nucleic acid sequence encoding an intracellular domain which comprises a signalling domain of the first CAR
   and
(ii) a second nucleic acid sequence encoding the second chimeric antigen receptor (CAR) as defined above, which nucleic acid sequence has the following structure:
   AgB2-spacer2-TM2-HD2 or AgB2-spacer2-TM2-endo2
   in which
   AgB2 is a nucleic acid sequence encoding the antigen-binding domain of the second CAR;
   spacer 2 is a nucleic acid sequence encoding the spacer of the second CAR;
   TM2 is a nucleic acid sequence encoding the transmembrane domain of the second CAR;
   HD2 is a nucleic acid sequence encoding a heterodimerisation domain of the second CAR;
   and
   Endo2 is a nucleic acid sequence encoding an intracellular domain which comprises a signalling domain of the second CAR.

The kit may also comprise (iii) a third nucleic acid sequence encoding an intracellular signalling molecule as defined above.

The kit may comprise: a first vector which comprises a first nucleic acid sequence as defined above; and a second vector which comprises a second nucleic acid sequence as defined above; and optionally a third vector which comprises a third nucleic acid sequence as defined above.

In a fourth aspect the present invention provides a vector comprising a nucleic acid construct according to the second aspect of the invention.

The vector or kit of vectors may, for example, be retroviral vector(s), lentiviral vector(s) or transposon(s).

In a fifth aspect, there is provided a method for making a cell according to the first aspect of the invention, which comprises the step of introducing: a nucleic acid construct according to the second aspect of the invention; a kit according to the third aspect of the invention; or a vector according to the fourth aspect of the invention, into a cell.

The cell may be from a sample isolated from a subject.

In a sixth aspect, the present invention provides a pharmaceutical composition comprising a plurality of cells according to the first aspect of the invention.

In a seventh aspect, there is provided a method for treating and/or preventing a disease, which comprises the step of administering a pharmaceutical composition according to the sixth aspect of the invention to a subject.

The method may comprise the following steps:
(i) isolation of a cell-containing sample from a subject;
(ii) transduction or transfection of the cells with: a nucleic acid construct according to the second aspect of the invention; a kit according to the third aspect of the invention; or a vector according to the fourth aspect of the invention; and
(iii) administering the cells from (ii) to a the subject.

The method may involve monitoring toxic activity in the subject and may comprise the step of administering an agent to the subject in order to reduce adverse toxic effects. The agent causes dissociation of the tunable CAR and the intracellular signalling molecule, as discussed above.

The disease may be a cancer, such as a B cell malignancy.

There is also provided a pharmaceutical composition according to the sixth aspect of the invention for use in treating and/or preventing a disease.

There is also provided the use of a cell according to the first aspect of the invention in the manufacture of a medicament for treating and/or preventing a disease.

There is also provided a method for inhibiting a tunable CAR system of a cell according to the first aspect of the invention, which comprises the step of administering an agent as defined above.

By providing one CAR which targets CD19 and one CAR which targets CD22, it is possible to target each of these markers, thereby reducing the problem of cancer escape.

By providing a single nucleic acid which encodes the two CARs separated by a cleavage site, it is possible to engineer cells to co-express the two CARs using a simple single transduction procedure. A double transfection procedure could be used with CAR-encoding sequences in separate constructs, but this would be more complex and expensive and requires more integration sites for the nucleic acids. A double transfection procedure would also be associated with uncertainty as to whether both CAR-encoding nucleic acids had been transduced and expressed effectively.

The CARs will have portions of high homology, for example the transmembrane and/or intracellular signalling domains are likely to be highly homologous. If the same or similar linkers are used for the two CARs, then they will also be highly homologous. This would suggest that an approach where both CARs are provided on a single nucleic acid sequence would be inappropriate, because of the likelihood of homologous recombination between the sequences. However, the present inventors have found that by “codon wobbling” the portions of sequence encoding areas of high homology, it is possible to express two CARs from a single construct with high efficiency. Codon wobbling involves using alternative codons in regions of sequence encoding the same or similar amino acid sequences.

By providing a “tunable” system, whereby signalling via the CD19 and/or CD22 CAR is/are controllable using an agent which disrupts the CAR signalling system, it is possible to reduce or block signalling if a CAR-related toxicity is observed. Since this inhibition is reversible by removal of the agent, it enables the clinician to control a toxicity without sacrificing the CAR-expressing cells in the patient.

By providing a system in which the CD19 CAR is “tunable” and either the CD22 is “classical” i.e. constitutively active in the presence of antigen, or the CD22 is tunable with a separate agent it is possible to tune down or turn off signalling via CD19 CAR engagement whilst maintaining signalling via CD22 CAR engagement. This provides a mechanism for controlling CD19 CAR-associated toxicity in the patient, whilst maintaining the anti-tumour effect via the CD22 CAR.

The presence of a coiled-coil spacer on the CD22 CAR causes multimerisation of the CD22 CAR at the cell surface. This enhances antigen recognition and signalling via the CD22 CAR, which is typically less than via a CD19 CAR due to the nature of the CD22 extracellular domain. Where the CD22 CAR comprises a 41BB co-stimulatory domain, the multimerisation enables constitutive 41BB signalling allowing progressive CAR-T cell accumulation and persistence in vivo.

### DESCRIPTION OF THE FIGURES

FIG. 1: a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2: B-cell maturation pathway/B-cell ontogeny. DR=HLA-DR; cCD79=cytoplasmic CD79; cCD22=cytoplasmic CD22. Both CD19 and CD22 antigens are expressed during early stages in B-cell maturation. It is these cells that develop into B-cell acute leukaemias. Targeting both CD19 as well as CD22 simultaneously is most suited for targeting B-cell acute leukaemias.

FIG. 3: Strategies for design of an anti-CD19 OR CD22 CAR cassette. Binders which recognize CD19 and binders which recognize CD22 are selected. An optimal spacer domain and signalling domain is selected for each CAR. (a) an OR gate cassette is constructed so that both CARs are co-expressed using a FMD-2A peptide. Any homologous sequences are codon-wobbled to avoid recombination. (c) The two CARs are co-expressed as separate proteins on the T-cell surface.

FIG. 4: Example of codon-wobbling to allow co-expression in a retroviral vector of identical peptide sequences but avoiding homologous recombination. Here, wild-type HCH2CH3-CD28tmZeta is aligned with codon-wobbled HCH2CH3-CD28tmZeta.

FIG. 5: Demonstrating functionality of anti-CD19 OR CD22 CAR gate. (a) Cartoon of construct: S1—signal peptide 1; HA—haemagglutin tag; HCH2CH3—hinge, CH2CH3 of IgG1 wild-type sequence; CD28tmZ—CD28 transmembrane domain and CD3ζ Zeta wobbled sequence; 2A—Foot and mouth disease 2A peptide; S2—signal peptide 2; V5—v5 epitope tag; aCD22—anti-CD22 scFv; HCH2CH3′—hinge, CH2CH3 of IgG1 wobbled sequence; CD28tmZ—CD28 transmembrane domain and CD3ζ Zeta wobbled sequence; (b) Co-expression of two receptors from a single vector. Peripheral blood T-cells were transduced with bicistronic vector after stimulation with OKT3 and anti-CD28. Cells were analysed five days after transduction by staining with anti-V5-FITC (invitrogen) and anti-HA-PE (abCam). The two CARs can be detected simultaneously on the T-cell surface. (c) Non-transduced T-cells, T-cells expressing just anti-CD19 CAR, T-cells expressing just anti-CD22 CAR and T-cells expressing the anti-CD19 OR CD22 CAR gate were challenged with target cells expressing neither CD19 or CD22, either CD19 or CD22 singly, or both antigen. T-cells expressing the anti-CD19 OR CD22 CAR gate could kill target cells even if one antigen was absent.

FIG. 6: Biacore affinity determination for murine CD22ALAb scFv, humanised CD22ALAb scFv and M971 scFv

FIG. 7: Biacore affinity determination for murine CD19ALAb scFv and humanised CD19ALAb

FIG. 8: Comparison of the binding kinetics between soluble scFv-CD19 binding for CD19ALAb scFv and fmc63 scFv

FIG. 9: Schematic diagram illustrating CD19ALAb CAR, fmc63 CAR, CD22ALAb CAR and M971 CAR used in the comparative studies FIG. 10: Killing assay of CD19 positive target cells comparing a CAR with a CD19ALAb antigen binding domain and an equivalent CAR with an fmc63 binding domain.

FIG. 11: A) Killing assay of CD22 positive target cells comparing a CAR with a CD22ALAb antigen binding domain and an equivalent CAR with an M971 binding domain. B) Assay comparing IFNγ release following co-culture 1:1 with CD22 positive SupT1 cells

FIG. 12: CD19 structure and exons

FIG. 13: Schematic diagrams and construct maps illustrating the four constructs tested in Example 5. In the construct map, portions marked with are codon-wobbled. A: CD19 and CD22 CAR both have 41BB-CD3zeta compound endodomains; B: CD19 and CD22 CAR both have OX40-CD3zeta compound endodomains; C: CD19 CAR has 41BB-CD3zeta compound endodomain and CD22 CAR has CD28-CD3zeta compound endodomain; and D: CD19 CAR has OX40-CD3zeta compound endodomain and CD22 CAR has CD28-CD3zeta compound endodomain

FIG. 14: Target cell killing by “cells expressing the constructs shown in FIG. 13.

FIG. 15—Structures of TetR and TiP. (a) sequence of TiP attached at the amino-terminus of an arbitrary protein; (b) Crystallography derived structure of TiP interacting with TetR (from PDB 2NS8 and Luckner et al (J. Mol. Biol. 368, 780-790 (2007)). TiP can be seen engaged deep within the TetR homodimer associating with many of the residues tetracycline associates with.

FIG. 16—(a) A membrane spanning receptor component comprises an extracellular antigen-binding domain, a transmembrane domain and an intracellular linker to TetR. A separate molecule, the signalling component, comprises an intracellular protein which is generated by fusion of TiP to one or several T-cell signalling domains. In the absence of tetracycline or tetracycline analogues, the receptor and the signalling components interact and in the presence of cognate antigen the system signals. (b) In the presence of tetracycline or tetracycline analogues, TiP is displaced from TetR and the receptor can not transmit signals even in the presence of cognate antigen.

FIG. 17—Intracellular linker domain derived from CD4.

FIG. 18—Test construct with eGFP to demonstrate function of the system. (a) a bicistronic construct expressed as a single transcript which self-cleaves at the 2A site to yield: TiP fused to eGFP; and a CAR with TetR as its endodomain. (b) Fluorescent micrograph of SupT1 cells expressing this construct in the absence of tetracycline. The eGFP fluorescence can clearly be seen at the cell membrane; (c) Fluorescent micrograph of the same cells but now in the presence of tetracycline. Here, the eGFP is cytoplasmic showing that tetracycline has displaced TiP.

FIG. 19—Initial TetCAR construct and control (a) a bicistronic construct expressed as a single transcript which self-cleaves at the 2A site to yield: a signalling component which comprises TiP fused via a flexible linker to the endodomain of CD3-Zeta; and a receptor component which comprises a CD33 recognizing scFv, a spacer derived from the Fc domain of IgG1, a CD4 derived transmembrane and intracellular domain; and TetR. (b) a control was also constructed which was identical except TiP was absent from the signalling component. (c) annotated amino-acid sequence of the basic TetCAR is shown.

FIG. 20—Function of the initial TetR construct in comparison with control. (a) TetCAR was expressed in BW5 T-cells. These T-cells were challenged with wild-type SupT1 cells or SupT1 cells engineered to express CD33 in the absence of tetracycline or in the presence of increasing concentrations of tetracycline. T-cells challenged with wild-type SupT1 cells do not activate in either the presence or absence of Tetracyline; T-cells challenged with SupT1 cells expressing CD33 activate in the absence of Tetracycline, but activation is rapidly inhibited in the presence of tetracycline with activation fully inhibited in the presence of 100 nM of Tetracycline. (b) Control TetCAR which lacks the TiP domain was transduced into BW5. Once again, these T-cells were challenged with wild-type SupT1 cells or SupT1 cells engineered to express CD33 in the absence or in the presence of increasing concentration of Tetracycline. A lack of TiP element in the signalling component resulted in no signalling in any conditions.

FIG. 21—Dual tetR domain tetCARs. tetR is expressed as a single-chain with two TetRs attached together. If tetR domains with differing affinity for tetracycline (and hence TiP) are used, the kinetics of Tetracycline mediated displacement of TiP can modulate the levels of signalling.

FIG. 22—A tetCAR signalling system utilising a plurality of signalling components containing single endodomains. A single CAR is expressed with many different signalling components all of which comprise TiP at their amino terminus but a different individual signalling domain, in contrast to a compound signalling domain. These randomly interact with the receptor component. Lack of steric interaction between the different signalling domains and their second messengers improves their function.

FIG. 23—A tetCAR signalling system utilising a plurality of signalling components containing single endodomains and different TiP domains. Each signalling component comprises of an individual signalling domain. Each signalling component also comprises of a TiP, however each TiP has different affinities to the TetR domain. Hence the stoichiometry of the interactions between the CAR and the signalling domains can be varied. In the example shown, the signalling system is constructed such that OX40>CD3Zeta>CD28.

FIG. 24—A tetCAR signalling system utilising a plurality of receptor components and a plurality of signalling components, each signalling component containing a single endodomain.

FIG. 25—TetCAR signalling in primary cells (a) Different constructs tested: (i) Classic CAR; (ii) tetCAR; (iii) control tetCAR where TiP has been deleted. (b) non-transduced and SupT1.CD19 cells stained for CD19; (c) Non-transduced T-cells and T-cells transduced with the different CAR constructs stained with anti-Fc.

FIG. 26—Interferon-Gamma release from non-transduced T-cells, and T-cells transduced with the different CAR construct challenged ((i) Classical first generation CAR, (ii) tetCAR and (iii) control tetCAR), with SupT1 cells, SupT1.CD19 cells in different concentrations of Tetracyline.

FIG. 27—Killing of target cells. A chromium release assay was used to demonstrate killing of target cells (SupT1.CD19) in the absence of tetracycline. Key: (i)—regular CAR; (ii) tetCAR; (iii)—control tetCAR (no TiP on endodomain).

FIG. 28—Schematic diagram showing tunable CD19/CD22 OR gate: In this embodiment, the CD22 CAR is not tunable; and the CD19 CAR is tunable. Tetracycline causes dissociation of the CD19 CAR from the intracellular signalling molecule. The CD19CAR includes a co-stimulatory domain (OX40) between the transmembrane domain and the heterodimerization domain.

FIG. 29—Schematic diagram showing tunable CD19/CD22 OR gate:

In this embodiment, the CD22 CAR is not tunable; and the CD19 CAR is tunable. Tetracycline causes dissociation of the CD19 CAR from the intracellular signalling molecule. The intracellular signalling molecule includes a co-stimulatory domain (OX40) and a CD3 zeta domain.

FIG. 30—Schematic diagram showing tunable CD19/CD22 OR gate: In this embodiment, both the CD19 and CD22 CARs are tunable with the same agent. Tetracycline causes dissociation of the CD19 CAR and the CD22 CAR from the intracellular signalling molecule. The CD19 CAR and CD22 CAR both include a co-stimulatory domain between the transmembrane domain and the heterodimerization domain (here shown as OX40 and 41BB, but could both be OX40 or 41BB).

FIG. 31—Schematic diagram showing tunable CD19/CD22 OR gate: In this embodiment, both the CD19 and CD22 CARs are tunable with the same agent. Tetracycline causes dissociation of the CD19 CAR and the CD22 CAR from the intracellular signalling molecules. The cell may comprise one or more types of intracellular signalling molecule. Here two types are shown, one having an OX40 costimulatory domain and one having a 41BB costimulatory domain. If the heterodimerization domains are the same, then there will be random heterodimerization between the two CARs and the two intracellular signalling molecules. If the two heterodimerization domain are different then the CD19 CAR will heterodimerise with the intracellular signalling molecule having the reciprocal heterodimerization domain and vice versa.

FIG. 32—Schematic diagram showing tunable CD19/CD22 OR gate: In this embodiment, both the CD19 and CD22 CARs are tunable, but with different agents. Tetracycline causes dissociation of the CD19 CAR from a first intracellular signalling molecule. Caffeine causes dissociation of CD22 CAR from a second intracellular signalling molecule. The CD19 CAR and CD22 CAR both include a co-stimulatory domain between the transmembrane domain and the heterodimerization domain (here shown as OX40 and 41BB, but could both be OX40 or 41BB).

FIG. 33—Schematic diagram showing tunable CD19/CD22 OR gate: In this embodiment, both the CD19 and CD22 CARs are tunable, but with different agents. Tetracycline causes dissociation of the CD19 CAR from a first intracellular signalling molecule. Caffeine causes dissociation of CD22 CAR from a second intracellular signalling molecule. The first intracellular signalling molecule comprises a first co-stimulatory domain (here shown as OX40) and the second intracellular signalling molecule comprises a second co-stimulatory domain (here shown as 41BB).

FIG. 34—Schematic diagram showing tunable CD19/CD22 OR gate: In this embodiment, the CD22 CAR is not tunable; and the CD19 CAR is tunable. Tetracycline causes dissociation of the CD19 CAR from the intracellular signalling molecule. The CD19CAR includes a co-stimulatory domain (OX40) between the transmembrane domain and the heterodimerization domain. The CD22 CAR is multimeric and comprises a coiled-coil spacer domain (COMP).

FIG. 35—Schematic diagram showing tunable CD19/CD22 OR gate: In this embodiment, the CD22 CAR is not tunable; and the CD19 CAR is tunable. Tetracycline causes dissociation of the CD19 CAR from the intracellular signalling molecule. The intracellular signalling molecule includes a co-stimulatory domain (OX40) and a CD3 zeta domain. The CD22 CAR is multimeric and comprises a coiled-coil spacer domain (COMP).

FIG. 36A—Schematic diagram illustrating CD19 and CD22 extracellular domains

FIG. 36B—Schematic diagram illustrating CD22ALAb CAR with a COMP spacer domain and CD22ALAb CAR with a hinge spacer domain

FIG. 37—Chromium release assay to investigate killing of Raji target cells after co-culture with CD22ALAb-COMP with CD22ALAb-hinge CAR T cells.

FIG. 38—Interferon-Gamma release from non-transduced T-cells, and T-cells transduced with anti-CD22 CARs having different spacers at a 4:1 and 1:1 E:T ratio with Raji target cells after 72 hours co-culture.

FIG. 39—Construct map illustrating a CD19/CD22 OR gate having a coiled-coil spacer domain on the CD22 CAR. The CD19 CAR has a OX40-CD3zeta compound endodomain and CD22 CAR has a 41BB-CD3zeta compound endodomain. The CD19 CAR has a CD8 stalk spacer and the CD22 CAR has a coiled-coil spacer domain (Comp). The CD19 CAR may be tunable, giving an OR gate as shown schematically in FIG. 35. For a tunable OR gate, the construct may be tri-cistronic, encoding the tunable CD19 CAR; the intracellular signalling molecule; and the CD22 CAR having a coiled-coil spacer domain.

FIG. 40A—A schematic diagram illustrating a tunable CD19 CAR having an fmc63 antigen binding domain, a CD8 stalk spacer and a CD8 transmembrane domain. Tetracycline causes dissociation of the CD19 CAR from the intracellular signalling molecule. The intracellular signalling molecule includes a co-stimulatory domain (CD28) and a CD3 zeta. FIG. 40B—A graph showing percentage survival of CD19+ target cells following co-culture with the tunable CD19 CAR or the equivalent classical (non-tunable) CAR in the presence or absence of tetracycline.

FIG. 41—Graphs showing percentage survival of CD19+ target cells following co-culture with A) non-transduced PBMCs; B) PBMCs transduced with a classical (non-tunable) CD19 CAR; or C) PBMCs transduced with the tunable CD19 CAR illustrated in FIG. 40A at an 8:1 or 4:1 E:T ratio, in the presence of a range of concentrations of tetracycline (0 nM-1600 nM).

FIG. 42—Determining A whether the effect or Tet is reversible and the On-Off and Off-On rates Top: A schematic diagram illustrating the experimental outline for Example 13. Bottom: Graphs showing percentage survival of CD19+ target cells following co-culture with CAR T cells expressing the tunable CD19 CAR. Tunable aCD19CAR T cells were preincubated with CD19+ positive target cells (A) or with Tet (B) for 2 hours prior to a cytotoxicity assay in the presence or absence of Tet.

### DETAILED DESCRIPTION

Chimeric Antigen Receptors (CARS)

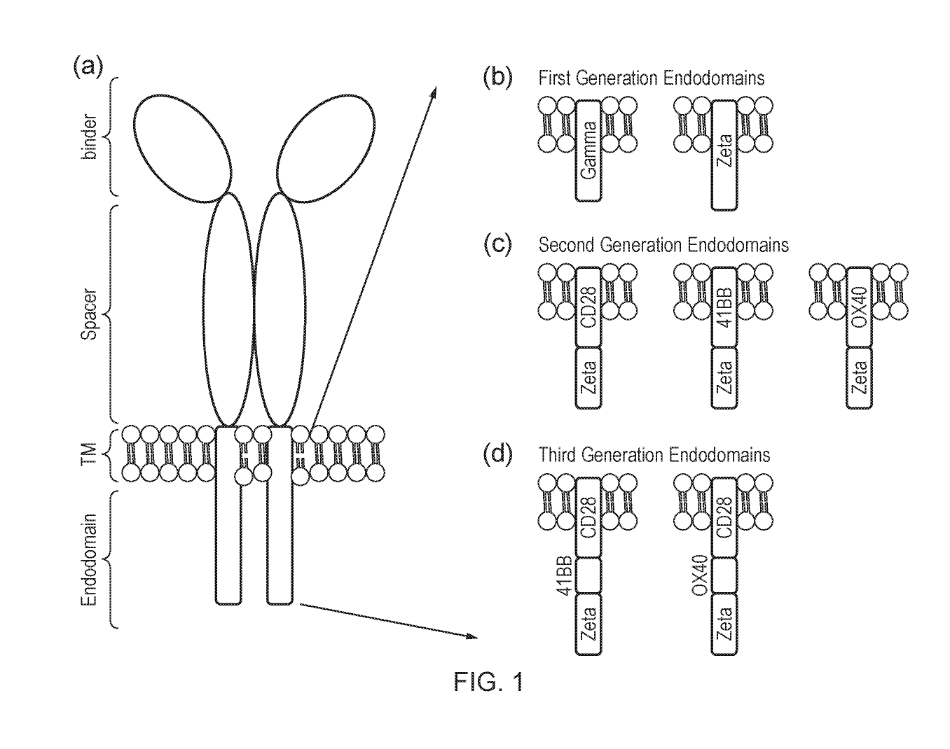

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The first aspect of the invention relates to a cell which co-expresses a first CAR and a second CAR, wherein one CAR binds CD19 and the other CAR binds CD22, such that a T-cell can recognize a target cells expressing either of these markers.

Thus, the antigen binding domains of the first and second CARs of the present invention bind to different antigens and both CARs may comprise an activating endodomain. The two CARs may comprise spacer domains which may be the same, or sufficiently different to prevent cross-pairing of the two different receptors. A cell can hence be engineered to activate upon recognition of either or both CD19 and CD22. This is useful in the field of oncology as indicated by the Goldie-Coldman hypothesis: sole targeting of a single antigen may result in tumour escape by modulation of said antigen due to the high mutation rate inherent in most cancers. By simultaneously targeting two antigens, the probably of such escape is exponentially reduced.

It is important that the two CARs do not heterodimerize.

The first and second CAR of the T cell of the present invention may be produced as a polypeptide comprising both CARs, together with a cleavage site.

The CARs of the cell of the present invention may comprise a signal peptide so that when the CAR is expressed inside a cell, such as a T-cell, the nascent protein is directed to the endoplasmic reticulum and subsequently to the cell surface, where it is expressed.

CD19

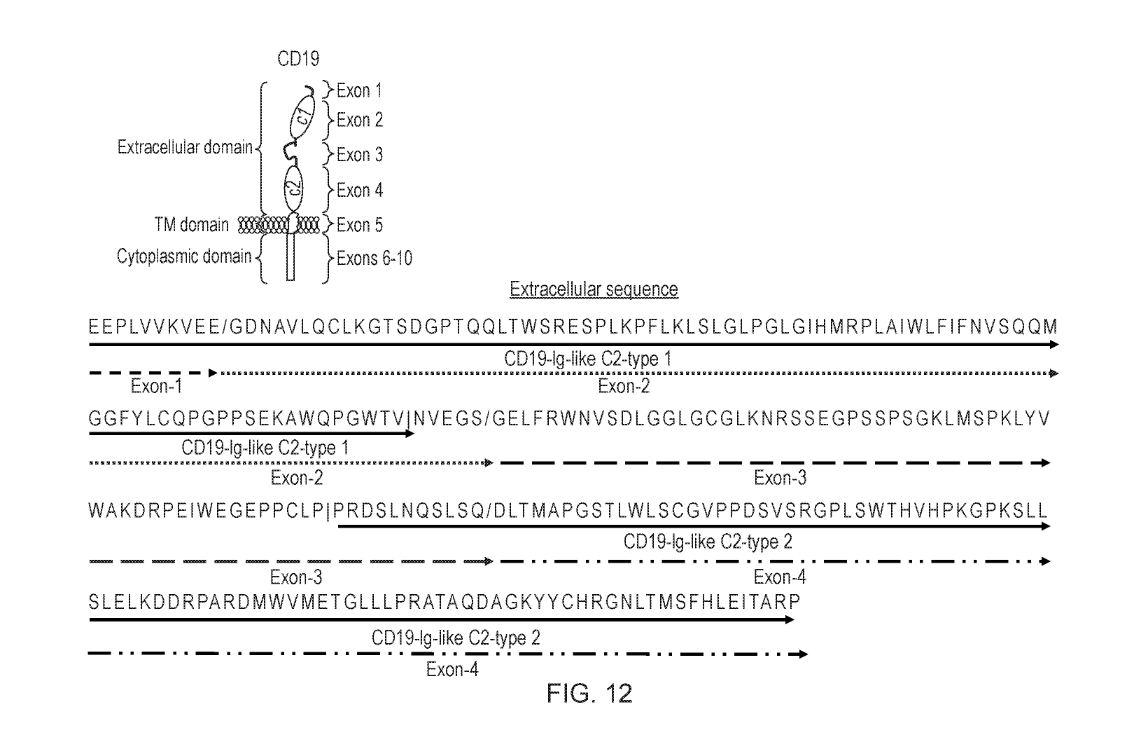

The human CD19 antigen is a 95 kd transmembrane glycoprotein belonging to the immunoglobulin superfamily. CD19 is classified as a type I transmembrane protein, with a single transmembrane domain, a cytoplasmic C-terminus, and extracellular N-terminus. The general structure for CD19 is illustrated in FIG. 12

CD19 is a biomarker for normal and neoplastic B cells, as well as follicular dendritic cells. In fact, it is present on B cells from earliest recognizable B-lineage cells during development to B-cell blasts but is lost on maturation to plasma cells. It primarily acts as a B cell co-receptor in conjunction with CD21 and CD81. Upon activation, the cytoplasmic tail of CD19 becomes phosphorylated, which leads to binding by Src-family kinases and recruitment of PI-3 kinase. CD19 is expressed very early in B-cell differentiation and is only lost at terminal B-cell differentiation into plasma cells. Consequently, CD19 is expressed on all B-cell malignancies apart from multiple myeloma.

Different designs of CARs have been tested against CD19 in different centres, as outlined in the following Table:

TABLE 1

| Centre | Binder | Endodomain | Comment |
|---|---|---|---|
| University College | Fmc63 | CD3-Zeta | Low-level brief |
| London | | | persistence |
| Memorial Sloane | SJ25C1 | CD28-Zeta | Short-term |
| Kettering | | | persistence |
| NCI/KITE | Fmc63 | CD28-Zeta | Long-term low-level |
| | | | persistence |
| Baylor, Centre for | Fmc63 | CD3-Zeta/ | Short-term low-level |
| Cell and Gene Therapy | | CD28-Zeta | persistence |
| UPENN/Novartis | Fmc63 | 41BB-Zeta | Long-term high-level |
| | | | persistence |

As shown above, most of the studies conducted to date have used an scFv derived from the hybridoma fmc63 as part of the binding domain to recognize CD19.

As shown in FIG. 12, the gene encoding CD19 comprises ten exons: exons 1 to 4 encode the extracellular domain; exon 5 encodes the transmembrane domain; and exons 6 to 10 encode the cytoplasmic domain,

In the CD19/CD22 OR gate of the present invention, the antigen-binding domain of the anti-CD19 CAR may bind an epitope of CD19 encoded by exon 1 of the CD19 gene.

In the CD19/CD22 OR gate of the present invention, the antigen-binding domain of the anti-CD19 CAR may bind an epitope of CD19 encoded by exon 3 of the CD19 gene.

In the CD19/CD22 OR gate of the present invention, the antigen-binding domain of the anti-CD19 CAR may bind an epitope of CD19 encoded by exon 4 of the CD19 gene.

CD19ALAb

The present inventors have developed a new anti-CD19 CAR which has improved properties compared to a known anti-CD19 CAR which comprises the binder fmc63 (see Examples 2 and 3). The antigen binding domain of the CAR is based on the CD19 binder CD19ALAb, which has the CDRs and VH/VL regions identified below.

The present invention therefore also provides a CAR which comprises a CD19-binding domain which comprises a) a heavy chain variable region (VH) having complementarity determining regions (CDRs) with the following sequences:

(SEQ ID No. 1)

CDR1 - SYWMN;

(SEQ ID No. 2)

CDR2 - QIWPGDGDTNYNGKFK

(SEQ ID No. 3)

CDR3 - RETTTVGRYYYAMDY;

and
b) a light chain variable region (VL) having CDRs with the following sequences:

(SEQ ID No. 4)

CDR1 - KASQSVDYDGDSYLN;

(SEQ ID No. 5)

CDR2 - DASNLVS

(SEQ ID No. 6)

CDR3 - QQSTEDPWT.

It may be possible to introduce one or more mutations (substitutions, additions or deletions) into the or each CDR without negatively affecting CD19-binding activity. Each CDR may, for example, have one, two or three amino acid mutations.

The CAR of the present invention may comprise one of the following amino acid sequences:

(Murine CD19ALAb scFv sequence)

SEQ ID No. 12

QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSSDIQLTQSPASLAVSLGQRATISC
KASQSVDYDGDSYLNWYQQIPGQPPKLLIYDASNLVSGIPPRFSGSGSG
TDFTLNIHPVEKVDAATYHCQQSTEDPWTFGGGTKLEIK
(Humanised CD19ALAb scFv sequence - Heavy 19,
Kappa 16)

SEQ ID No. 13

QVQLVQSGAEVKKPGASVKLSCKASGYAFSSYWMNVWVRQAPGQSLEWI
GQIWPGDGDTNYNGKFKGRATLTADESARTAYMELSSLRSGDTAVYFCA
RRETTTVGRYYYAMDYWGKGTLVTVSSDIQLTQSPDSLAVSLGERATIN
CKASQSVDYDGDSYLNWYQQKPGQPPKLLIYDASNLVSGVPDRFSGSGS
GTDFTLTISSLQAADVAVYHCQQSTEDPWTFGQGTKVEIKR
(Humanised CD19ALAb scFv sequence - Heavy 19,
Kappa 7)

SEQ ID No. 14

QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSSDIQLTQSPDSLAVSLGERATINC
KASQSVDYDGDSYLNWYQQKPGQPPKVLIYDASNLVSGVPDRFSGSGSG
TDFTLTISSLQAADVAVYYCQQSTEDPWTFGQGTKVEIKR

The scFv may be in a VH-VL orientation (as shown in SEQ ID Nos 12, 13 and 14) or a VL-VH orientation.

The CAR of the present invention may comprise one of the following VH sequences:

(Murine CD19ALAb VH sequence)

SEQ ID No. 7

QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCARR
ETTTVGRYYYAMDYWGQGTTVTVSS
(Humanised CD19ALAb VH sequence)

SEQ ID No. 8

QVQLVQSGAEVKKPGASVKLSCKASGYAFSSYWMNVWVRQAPGQSLE
WIGQIWPGDGDTNYNGKFKGRATLTADESARTAYMELSSLRSGDTAVYF
CARRETTTVGRYYYAMDYWGKGTLVTVSS

The CAR of the present invention may comprise one of the following VL sequences:

(Murine CD19ALAb VL sequence)

SEQ ID No. 9

DIQLTQSPASLAVSLGQRATISCKASQSVDYDGDSYLNWYQQIPGQPP
KLLIYDASNLVSGIPPRFSGSGSGTDFTLNIHPVEKVDAATYHCQQSTE
DPWTFGGGTKLEIK
(Humanised CD19ALAb VL sequence, Kappa 16)

SEQ ID No. 10

DIQLTQSPDSLAVSLGERATINCKASQSVDYDGDSYLNWYQQKPGQP
PKLLIYDASNLVSGVPDRFSGSGSGTDFTLTISSLQAADVAVYHCQQS
TEDPWTFGQGTKVEIKR
(Humanised CD19ALAb VL sequence, Kappa 7)

SEQ ID No. 11

DIQLTQSPDSLAVSLGERATINCKASQSVDYDGDSYLNWYQQKPGQP
PKVLIYDASNLVSGVPDRFSGSGSGTDFTLTISSLQAADVAVYYCQQS
TEDPWTFGQGTKVEIKR

The CAR of the invention may comprise a variant of the sequence shown as SEQ ID No. 7 to 14 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at http://blast.ncbi.nlm.nih.gov.

CD22

The human CD22 antigen is a molecule belonging to the SIGLEC family of lectins. It is found on the surface of mature B cells and on some immature B cells. Generally speaking,

CD22 is a regulatory molecule that prevents the overactivation of the immune system and the development of autoimmune diseases.

CD22 is a sugar binding transmembrane protein, which specifically binds sialic acid with an immunoglobulin (Ig) domain located at its N-terminus. The presence of Ig domains makes

CD22 a member of the immunoglobulin superfamily. CD22 functions as an inhibitory receptor for B cell receptor (BCR) signaling.

CD22 is a molecule of the IgSF which may exist in two isoforms, one with seven domains and an intra-cytoplasmic tail comprising of three ITIMs (immune receptor tyrosine-based inhibitory motifs) and an ITAM; and a splicing variant which instead comprises of five extracellular domains and an intra-cytoplasmic tail carrying one ITIM. CD22 is thought to be an inhibitory receptor involved in the control of B-cell responses to antigen. Like CD19, CD22 is widely considered to be a pan-B antigen, although expression on some non-lymphoid tissue has been described. Targeting of CD22 with therapeutic monoclonal antibodies and immunoconjugates has entered clinical testing.

Examples of anti-CD22 CARs are described by Haso et al. (Blood; 2013; 121(7)). Specifically, anti-CD22 CARs with antigen-binding domains derived from m971, HA22 and BL22 scFvs are described.

The antigen-binding domain of the anti-CD22 CAR may bind CD22 with a K_{D} in the range 30-50 nM, for example 30-40 nM. The K_{D} may be about 32 nM.

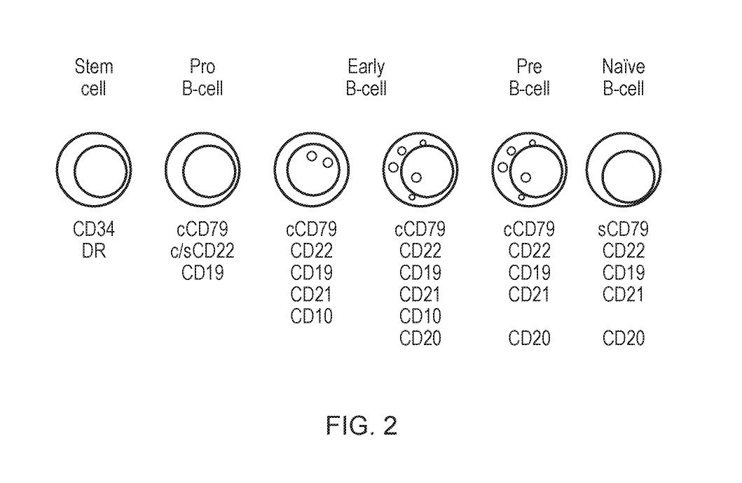

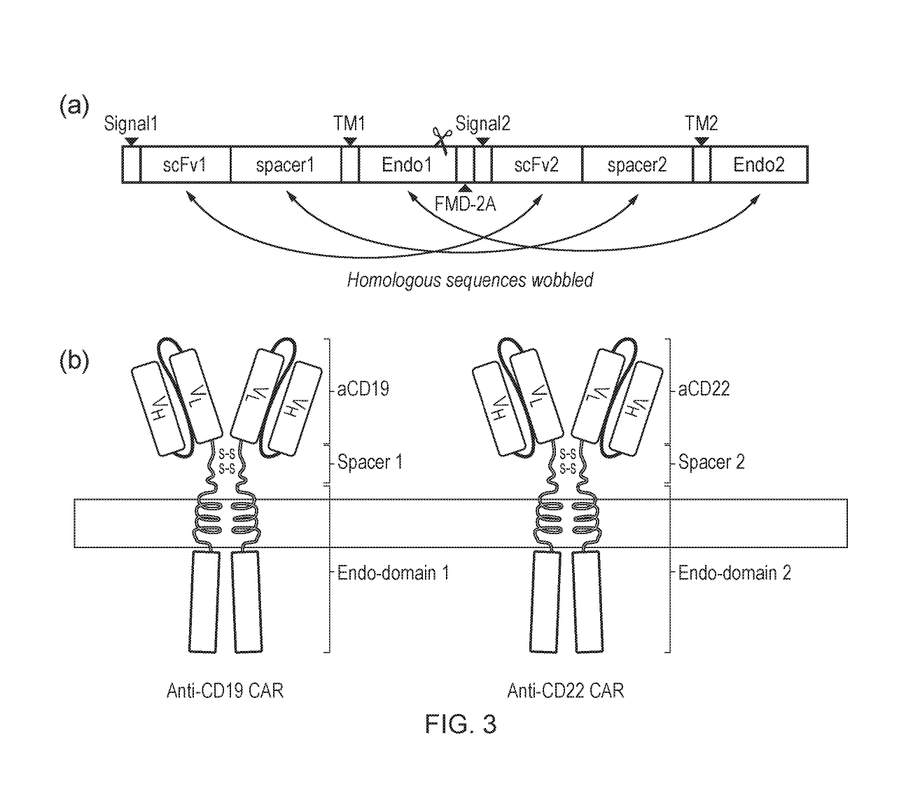

CD-22 has seven extracellular IgG-like domains, which are commonly identified as Ig domain 1 to Ig domain 7, with Ig domain 7 being most proximal to the B cell membrane and Ig domain 7 being the most distal from the Ig cell membrane (see Haso et al 2013 as above FIG. 2B).

The positions of the Ig domains in terms of the amino acid sequence of CD22 (http://www.uniprot.org/uniprot/P20273) are summarised in the following table:

| Ig domain | Amino acids |
|---|---|
| 1 | 20-138 |
| 2 | 143-235 |
| 3 | 242-326 |
| 4 | 331-416 |
| 5 | 419-500 |
| 6 | 505-582 |
| 7 | 593-676 |

The antigen-binding domain of the second CAR may bind to a membrane-distal epitope on CD22. The antigen-binding domain of the second CAR may bind to an epitope on Ig domain 1, 2, 3 or 4 of CD22, for example on Ig domain 3 of CD22. The antigen-binding domain of the second CAR may bind to an epitope located between amino acids 20-416 of CD22, for example between amino acids 242-326 of CD22.

The anti-CD22 antibodies HA22 and BL22 (Haso et al 2013 as above) and CD22ALAb, described below, bind to an epitope on Ig domain 3 of CD22.

The antigen binding domain of the second CAR may not bind to a membrane-proximal epitope on CD22. The antigen-binding domain of the second CAR may not bind to an epitope on Ig domain 5, 6 or 7 of CD22. The antigen-binding domain of the second CAR may not bind to an epitope located between amino acids 419-676 of CD22, such as between 505-676 of CD22.

CD22ALAb

The present inventors have developed a new anti-CD22 CAR which has improved properties compared to a known anti-CD22 CAR which comprises the binder m971 (see Examples 2 and 3 and Haso et al (2013) as above). The antigen binding domain of the CAR is based on the CD22 binder CD22ALAb, which has the CDRs and VH/VL regions identified below.

The present invention therefore also provides a CAR which comprises a CD22-binding domain which comprises

a) a heavy chain variable region (VH) having complementarity determining regions (CDRs) with the following sequences:

(SEQ ID No. 15)

CDR1 - NYWIN;

(SEQ ID No. 16)

CDR2 - NIYPSDSFTNYNQKFKD

(SEQ ID No. 17)

CDR3 - DTQERSWYFDV;

and
b) a light chain variable region (VL) having CDRs with the following sequences:

(SEQ ID No. 18)

CDR1 - RSSQSLVHSNGNTYLH;

(SEQ ID No. 19)

CDR2 - KVSNRFS

(SEQ ID No. 20)

CDR3 - SQSTHVPWT.

It may be possible to introduce one or more mutations (substitutions, additions or deletions) into the or each CDR without negatively affecting CD22-binding activity. Each CDR may, for example, have one, two or three amino acid mutations.

The CAR of the present invention may comprise one of the following amino acid sequences:

(Murine CD22ALAb scFv sequence)

SEQ ID No. 25

QVQLQQPGAELVRPGASVKLSCKASGYTFTNYWINWVKQRPGQGLEWIG
NIYPSDSFTNYNQKFKDKATLTVDKSSSTAYMQLSSPTSEDSAVYYCT
RDTQERSWYFDVWGAGTTVTVSSDVVMTQTPLSLPVSLGDQASISCRS
SQSLVHSNGNTYLHWYLQKPGQSPKLLIYKVSNRFSGVPDRFSGSGSGT
DFTLKISRVEAEDLGLYFCSQSTHVPWTFGGGTKLEIK
(Humanised CD22ALAb scFv sequence)

SEQ ID No. 26

EVQLVESGAEVKKPGSSVKVSCKASGYTFTNYWINWVRQAPGQGLEWIG
NIYPSDSFTNYNQKFKDRATLTVDKSTSTAYLELRNLRSDDTAVYYCTR
DTQERSWYFDVWGQGTLVTVSSDIVMTQSPATLSVSPGERATLSCRSSQ
SLVHSNGNTYLHWYQQKPGQAPRLLIYKVSNRFSGVPARFSGSGSGVEF
TLTISSLQSEDFAVYYCSQSTHVPWTFGQGTRLEIK

The scFv may be in a VH-VL orientation (as shown in SEQ ID Nos 25 and 26) or a VL-VH orientation.

The CAR of the present invention may comprise one of the following VH sequences:

(Murine CD22ALAb VH sequence)

SEQ ID No. 21

QVQLQQPGAELVRPGASVKLSCKASGYTFTNYWINWVKQRPGQGLEWIG
NIYPSDSFTNYNQKFKDKATLTVDKSSSTAYMQLSSPTSEDSAVYYCTR
DTQERSWYFDVWGAGTTVTVSS
(Humanised CD22ALAb VH sequence)

SEQ ID No. 22

EVQLVESGAEVKKPGSSVKVSCKASGYTFTNYWINWVRQAPGQGLEWIG
NIYPSDSFTNYNQKFKDRATLTVDKSTSTAYLELRNLRSDDTAVYYCTR
DTQERSWYFDVWGQGTLVTVSS

The CAR of the present invention may comprise one of the following VL sequences:

(Murine CD22ALAb VL sequence)

SEQ ID No. 23

DVVMTQTPLSLPVSLGDQASISCRSSQSLVHSNGNTYLHWYLQKPGQSPK
LLIYKVSNRFSGVPDRFSGSGSGTDFTLKISRVEAEDLGLYFCSQSTHVP
WTFGGGTKLEIK
(Humanised CD22ALAb VL sequence)

SEQ ID No. 24

DIVMTQSPATLSVSPGERATLSCRSSQSLVHSNGNTYLHWYQQKPGQAPR
LLIYKVSNRFSGVPARFSGSGSGVEFTLTISSLQSEDFAVYYCSQSTHVP
WTFGQGTRLEIK

The CAR of the invention may comprise a variant of the sequence shown as SEQ ID No. 21 to 26 having at least 80, 85, 90, 95, 98 or 99% sequence identity, provided that the variant sequence retain the capacity to bind CD22 (when in conjunction with a complementary VL or VH domain, if appropriate).

B-Cell Antigen Expression During B-Cell Ontogeny and Subsequent Tumours

CD19 is widely considered a pan-B antigen, although very occasionally, it may display some lineage infidelity. The CD19 molecule comprises of two extracellular IgSF domains separated by a smaller domain and a long intracytoplasmic tail, nearly as big as the extracellular portion of the molecule, carrying one ITAM. CD19 is a key molecule in the development and activation of B-cells. CD22 is a molecule of the IgSF which may exist in two isoforms, one with seven domains and an intra-cytoplasmic tail comprising of three ITIMs (immune receptor tyrosine-based inhibitory motifs) and an ITAM; and a splicing variant which instead comprises of five extracellular domains and an intra-cytoplasmic tail carrying one ITIM. CD22 is thought to be an inhibitory receptor involved in the control of B-cell responses to antigen. Like CD19, CD22 is widely considered to be a pan-B antigen, although expression on some non-lymphoid tissue has been described (Wen et al. (2012) J. Immunol. Baltim. Md. 1950 188, 1075-1082). Targeting of CD22 with therapeutic monoclonal antibodies and immunoconjugates has entered clinical testing. Generation of CD22 specific CARs have been described (Haso et al, 2013, Blood: Volume 121; 7: 1165-74, and James et al 2008, Journal of immunology, Volume 180; Issue 10; Pages 7028-38).

Detailed immunophentyping studies of B-cell leukaemias shows that while surface CD19 is always present, surface CD22 is almost always present. For instance, Raponi et al (2011, as above) studied the surface antigen phenotype of 427 cases of B-ALL and found CD22 present in 341 of cases studied.

The eventuality of CD19 down-regulation after CAR19 targeting described above may be explained by the Goldie-Coldman hypothesis. The Goldie-Coldman hypothesis predicts that tumor cells mutate to a resistant phenotype at a rate dependent on their intrinsic genetic instability and that the probability that a cancer would contain resistant clones depends on the mutation rate and the size of the tumor. While it may be difficult for cancer cells to become intrinsically resistant to the direct killing of cytotoxic T-cells, antigen loss remains possible. Indeed this phenomenon has been reported before with targeting melanoma antigens and EBV-driven lymphomas. According to Goldie-Coldman hypothesis, the best chance of cure would be to simultaneously attack non-cross resistant targets. Given that CD22 is expressed on nearly all cases of B-ALL, simultaneous CAR targeting of CD19 along with CD22 may reduce the emergence of resistant CD19 negative clones.

Tunable Car

The present invention relates to a tunable CAR system, composed of two components: (i) a CAR having an intracellular domain which comprises a heterodimerization domain, and (ii) a separate intracellular signalling molecule which comprises a reciprocal heterodimerization domain and a signalling domain.

The present invention provides a cell comprising a CAR system in which the antigen-recognizing/antigen binding domain and transmembrane domain of the tunable CAR are provided on a first molecule (termed herein ‘receptor component’), which localizes to the cell membrane. The intracellular signalling domain is provided on a second, intracellular molecule (termed herein ‘signalling component’).

Importantly, the receptor component comprises a first binding domain and the signalling component comprises a second binding domain which specifically binds to the first binding domain of the receptor component. Thus binding of the first binding domain to the second binding domain causes heterodimerization and co-localization of the receptor component and the signalling component. When antigen binds to the antigen binding domain of the receptor component there is signalling through the signalling component.

The first or second binding domain is also capable of binding a further agent in addition to the reciprocal binding domain. The further agent may be, for example, a small molecule. The binding between the agent and the first or second binding domain is of a higher affinity than the binding between the first binding domain and the second binding domain. Thus, when the agent is present it preferentially binds to the first or second binding domain and inhibits/disrupts the heterodimerization between the receptor component and the signalling component. When antigen binds to the antigen binding domain of the receptor component in the presence of the further agent there is no signalling through the signalling component.

Specifically, in the presence of the agent, the receptor component and signalling component are located in a stochastically dispersed manner and binding of antigen by the antigen-binding domain of the receptor component does not result in signalling through the signaling component.

Herein ‘co-localization’ or ‘heterodimerization’ of the receptor and signalling components is analogous to ligation/recruitment of the signalling component to the receptor component via binding of the first binding domain of the receptor component and the second binding domain of the signalling component.

Antigen binding by the receptor component in the presence of the agent may be termed as resulting in ‘non-productive’ signalling through the signalling component. Such signalling does not result in cell activation, for example T cell activation. Antigen binding by the receptor component in the absence of the agent may be termed as resulting in ‘productive’ signalling through the signalling component. This signalling results in T-cell activation, triggering for example target cell killing and T cell activation.

Antigen binding by the receptor component in the absence of the agent may result in signalling through the signalling component which is 2, 5, 10, 50, 100, 1,000 or 10,000-fold higher than the signalling which occurs when antigen is bound by the receptor component in the presence of the agent.

Signalling through the signalling component may be determined by a variety of methods known in the art. Such methods include assaying signal transduction, for example assaying levels of specific protein tyrosine kinases (PTKs), breakdown of phosphatidylinositol 4,5-biphosphate (PIP2), activation of protein kinase C (PKC) and elevation of intracellular calcium ion concentration. Functional readouts, such as clonal expansion of T cells, upregulation of activation markers on the cell surface, differentiation into effector cells and induction of cytotoxicity or cytokine secretion may also be utilised. As an illustration, in the present examples the inventors determined levels of interleukin-2 (IL-2) produced by T-cells expressing a receptor component and signalling component of a tunable CAR system upon binding of antigen to the receptor component in the presence of varying concentrations of an agent.

First Binding Domain, Second Binding Domain and Agent

The first binding domain, second binding domain and agent of the tunable CAR system may be any combination of molecules/peptides/domains which enable the selective co-localization and dimerization of the receptor component and signalling component in the absence of the agent.

The first binding domain and second binding domain are capable of specifically binding.

The signalling system of the present invention is not limited by the arrangement of a specific dimerization system. The receptor component may comprise either the first binding domain or the second binding domain of a given dimerization system so long as the signalling component comprises the corresponding, complementary binding domain which enables the receptor component and signalling component to co-localize in the absence of the agent.

The first binding domain and second binding domain may be a peptide domain and a peptide binding domain; or vice versa. The peptide domain and peptide binding domain may be any combination of peptides/domains which are capable of specific binding.

The agent is a molecule, for example a small molecule, which is capable of specifically binding to the first binding domain or the second binding domain at a higher affinity than the binding between the first binding domain and the second binding domain.

The binding system may be based on a peptide:peptide binding domain system. The first or second binding domain may comprise the peptide binding domain and the other binding domain may comprise a peptide mimic which binds the peptide binding domain with lower affinity than the peptide. The use of peptide as agent disrupts the binding of the peptide mimic to the peptide binding domain through competitive binding. The peptide mimic may have a similar amino acid sequence to the “wild-type” peptide, but with one of more amino acid changes to reduce binding affinity for the peptide binding domain.

The agent may bind the first binding domain or the second binding domain with at least 10, 20, 50, 100, 1000 or 10000-fold greater affinity than the affinity between the first binding domain and the second binding domain.

The agent may be any pharmaceutically acceptable molecule which preferentially binds the first binding domain or the second binding domain with a higher affinity than the affinity between the first binding domain and the second binding domain.

The agent is capable of being delivered to the cytoplasm of a target cell and being available for intracellular binding.

The agent may be capable of crossing the blood-brain barrier.

Small molecule systems for controlling the co-localization of peptides are known in the art, for example the Tet repressor (TetR), TetR interacting protein (TiP), tetracycline system (Klotzsche et al.; J. Biol. Chem. 280, 24591-24599 (2005); Luckner et al.; J. Mol. Biol. 368, 780-790 (2007)).

The Tet Repressor (TetR) System

The Tet operon is a well-known biological operon which has been adapted for use in mammalian cells. The TetR binds tetracycline as a homodimer and undergoes a conformational change which then modulates the DNA binding of the TetR molecules.

Klotzsche et al. (as above), described a phage-display derived peptide which activates the TetR. This protein (TetR interacting protein/TiP) has a binding site in TetR which overlaps, but is not identical to, the tetracycline binding site (Luckner et al.; as above). Thus TiP and tetracycline compete for binding of TetR.

In the tunable CAR system, the first binding domain of the receptor component may be TetR or TiP, provided that the second binding domain of the signalling component is the corresponding, complementary binding partner. For example if the first binding domain of the receptor component is TetR, the second binding domain of the signalling component is TiP. If the first binding domain of the receptor component is TiP, the second binding domain of the signalling component is TetR.

For example, the first binding domain or second binding domain may comprise the sequence shown as SEQ ID NO: 27 or SEQ ID NO: 28:

TetR

SEQ ID NO: 27

MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGH
TiP

SEQ ID NO: 28

MWTWNAYAFAAPSGGGS

TetR must homodimerize in order to function. Thus when the first binding domain on the receptor component is TetR, the receptor component may comprise a linker between the transmembrane domain and the first binding domain (TetR). The linker enables TetR to homodimerize with a TetR from a neighbouring receptor component and orient in the correct direction.

The linker may be the sequence shown as SEQ ID NO: 29.

modified CD4 endodomain

SEQ ID NO: 29

ALIVLGGVAGLLLFIGLGIFFCVRCRHRRRQAERMAQIKRVVSEKKTAQA
PHRFQKTCSPI

The linker may alternatively comprise an alternative linker sequence which has similar length and/or domain spacing properties as the sequence shown as SEQ ID NO: 29.

The linker may have at least 80%, 85%, 90%, 95%, 98% or 99% sequence identity to SEQ ID NO: 29 providing it provides the function of enabling TetR to homodimerize with a TetR from a neighbouring receptor component and orient in the correct direction.

One potential disadvantage of the TetR/TiP system is TetR is xenogenic and immunogenic.

The TetR sequence may therefore be a variant which is less immunogenic but retains the ability to specifically bind TiP.

Where the first and second binding domains are TetR or TiP or a variant thereof, the agent may be tetracycline, doxycycline, minocycline or an analogue thereof.

An analogue refers to a variant of tetracycline, doxycycline or minocycline which retains the ability to specifically bind to TetR.

Other combinations of binding domains and agents which may be used in the present CAR system are known in the art. For example, the CAR system may use a streptavidin/biotin-based binding system.

Streptavidin-Binding Epitope

The first or second binding domain may comprise one or more streptavidin-binding epitope(s). The other binding domain may comprise a biotin mimic.

Streptavidin is a 52.8 kDa protein from the bacterium Streptomyces avidinii. Streptavidin homo-tetramers have a very high affinity for biotin (vitamin B7 or vitamin H), with a dissociation constant (Kd)˜10-15 M. The biotin mimic has a lower affinity for streptavidin than wild-type biotin, so that biotin itself can be used as the agent to disrupt or prevent heterodimerisation between the streptavidin domain and the biotin mimic domain. The biotin mimic may bind streptavidin with for example with a Kd of 1 nM to 100 uM.

The ‘biotin mimic’ domain may, for example, comprise a short peptide sequence (for example 6 to 20, 6 to 18, 8 to 18 or 8 to 15 amino acids) which specifically binds to streptavidin.

The biotin mimic may comprise a sequence as shown in Table 2.

TABLE 2
Biotin mimicking peptides

| name | Sequence | affinity |
|---|---|---|
| Long nanotag | DVEAWLDERVPLVET (SEQ ID NO: 30) | 3.6 nM |
| Short nanotag | DVEAWLGAR (SEQ ID NO: 31) | 17 nM |
| Streptag | WRHPQFGG (SEQ ID NO: 32) | 72 uM |
| streptagII | WSHPQFEK (SEQ ID NO: 33) | |
| SBP-tag | MDEKTTGWRGGHVVEGLAGELEQLRARLEHHPQGQREP | 2.5 nM |
| | (SEQ ID NO: 34) | |
| ccstreptag | CHPQGPPC (SEQ ID NO: 35) | 230 nM |
| flankedccstreptag | AECHPQGPPCIEGRK (SEQ ID NO: 36) | |

The biotin mimic may be selected from the following group: Streptagll, Flankedccstreptag and ccstreptag.

The streptavidin domain may comprise streptavidin having the sequence shown as SEQ ID No. 37 or a fragment or variant thereof which retains the ability to bind biotin.

Full length Streptavidin has 159 amino acids. The N and C termini of the 159 residue full-length protein are processed to give a shorter ‘core’ streptavidin, usually composed of residues 13-139; removal of the N and C termini is necessary for the high biotin-binding affinity.

The sequence of “core” streptavidin (residues 13-139) is shown as SEQ ID No. 37

SEQ ID No. 37

EAGITGTWYNQLGSTFIVTAGADGALTGTYESAVGNAESRYVLTGRYDSA
PATDGSGTALGWTVAWKNNYRNAHSATTWSGQYVGGAEARINTQWLLTSG
TTEANAWKSTLVGHDTFTKVKPSAAS

Streptavidin exists in nature as a homo-tetramer. The secondary structure of a streptavidin monomer is composed of eight antiparallel β-strands, which fold to give an antiparallel beta barrel tertiary structure. A biotin binding-site is located at one end of each β-barrel. Four identical streptavidin monomers (i.e. four identical β-barrels) associate to give streptavidin's tetrameric quaternary structure. The biotin binding-site in each barrel consists of residues from the interior of the barrel, together with a conserved Trp120 from neighbouring subunit. In this way, each subunit contributes to the binding site on the neighbouring subunit, and so the tetramer can also be considered a dimer of functional dimers.

The streptavidin domain of the CAR system of the present invention may consist essentially of a streptavidin monomer, dimer or tetramer.

The sequence of the streptavidin monomer, dimer or tetramer may comprise all or part of the sequence shown as SEQ ID No. 37, or a variant thereof which retains the capacity to bind biotin.

A variant streptavidin sequence may have at least 70, 80, 90, 95 or 99% identity to SEQ ID No. 37 or a functional portion thereof. Variant streptavidin may comprise one or more of the following amino acids, which are involved in biotin binding: residues Asn23, Tyr43, Ser27, Ser45, Asn49, Ser88, Thr90 and Asp128. Variant streptavidin may, for example, comprise all 8 of these residues. Where variant streptavidin is present in the binding domain as a dimer or tetramer, it may also comprise Trp120 which is involved in biotin binding by the neighbouring subunit.

Single Domain Binders

The tunable CAR may comprise a single domain binder and the intracellular signalling molecule may comprise a binding domain which binds the single domain binder. Alternatively the intracellular signalling molecule may comprise a single domain binder and the tunable CAR may comprise a binding domain which binds the single domain binder.

A “single domain binder” is an entity which binds to an agent, such as a small molecule agent, and has a single domain. A protein domain has a compact three-dimensional structure. It may be derivable from a larger protein, but the domain itself is independently stable and folds independently.

The single domain binder may have an antibody-like binding site which binds to the agent. The single domain binder may comprise one or more complementarity determining regions (CDRs). The single domain binder may comprise three CDRs

The single domain binder may lack disulphide bonds. The single domain binder may lack cysteine residues.

A conventional IgG molecule is comprised of two heavy and two light chains. Heavy chains comprise three constant domains and one variable domain (VH); light chains comprise one constant domain and one variable domain (VL). The naturally functional antigen binding unit is formed by noncovalent association of the VH and the VL domain. This association is mediated by hydrophobic framework regions. IgG can be derivatized to Fab, scFv, and single domain VH or VL binders. The single domain binder used in the CAR system of the invention may be or comprise such a single domain VH or VL binder.

Heavy chain antibodies (hcAb) are found in Camelidae, lack the light chain and the CH1 domain. They comprise a single, antigen binding domain, the VHH domain. The single domain binder used in the CAR system of the invention may be or comprise such a VHH domain or derivative thereof.

A variety of non-immunoglobulin single domain binders have also been designed and characterised, including those based on natural and synthetis protein scaffolds. For example, fibronectin-derived Adnectins/monobodies are characterized by an Ig-like β-sandwich structure, anticalins are based on the lipocalin fold, affibodies derive from protein A and comprise three a helices, and DARPins are designer proteins composed of ankyrin repeats. Each design includes randomized residues that mediate ligand binding.

The single domain binder may have a molecular weight (when considered separately from the rest of the receptor component or signalling component of less than 20 kDa. It may, for example have a molecular weight of less than or equal to approximately 15 kDa, such as between 12-15 kDa, the typical molecular weight of a single domain antibody. Single chain variable fragments, which comprise two variable domains, VH and VL) typically have a molecular weight of about 25 kDa.

The single domain binder may be less than 150 amino acids in length, for example, less than 140, 130 or 120 amino acids in length. The single domain binder may be approximately 110 amino acids in length, for example from 105-115 amino acids in length

The single domain binder used in the CAR system of the invention may be a single domain antibody (sdAb, also known as a nanobody), an affibody, a fibronectin artificial antibody scaffold, an anticalin, an affilin, a DARPin, a VNAR, an iBody, an affimer, a fynomer, a domain antibody (DAb), an abdurin/nanoantibody, a centyrin, an alphabody or a nanofitin.

A single-domain antibody is an antibody fragment consisting of a single monomeric variable antibody domain. The first single-domain antibodies were engineered from heavy-chain antibodies found in camelids; i.e. VHH fragments. Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, Nanobodies derived from light chains have also been shown to bind specifically to target epitopes.

A single-domain antibody can be obtained by immunization of dromedaries, camels, llamas, alpacas or sharks with the desired antigen and subsequent isolation of the mRNA coding for heavy-chain antibodies. By reverse transcription and polymerase chain reaction, a gene library of single-domain antibodies may be produced. Screening techniques like phage display and ribosome display help to identify the clones binding the antigen.

Heterodimerisation of the tunable CAR and signalling component may occur through the binding of the single domain binder with a single domain binder-interacting peptide (sdbiP).

The sdbiP may, for example, be between 8-30, for example 10-20 amino acids in length.

Suitable sdbiPs may be generated and identified using peptide display methods such as phage display, CIS display, ribosome display and mRNA display (Ullman et al (2011) Briefings in Functional Genomics 10:125-134).

Peptides in a phage display peptide library may be selected using techniques such as biopanning (Miura et al (2004) Biochim. Et Biophys. Acta 1673:131-138).

The agent itself may be used to elute the peptides, for example in a peptide array, so that the selection method reflects the properties of the sdbiP in the CAR signalling system, namely that it binds the single domain binder, but the binding is competitively inhibited by the presence of the agent.

The agent for use with a single domain binder may be a small molecule such as: a steroid, methotrexate, caffeine, cocaine or an antibiotic.

Disruptable Protein:Protein Interations

Small molecules agents which disrupt protein-protein interactions have long been developed for pharmaceutical purpose (reviewed by Vassilev et al; Small-Molecule Inhibitors of Protein-Protein Interactions ISBN: 978-3-642-17082-9). A CAR system as described may use such a small molecule. The proteins or peptides whose interaction is disrupted (or relevant fragments of these proteins) can be used as the first and/or second binding domains and the small molecule may be used as the agent which inhibits CAR activation. Such a system may be varied by altering the small molecule and proteins such the system functions as described but the small molecule is devoid of unwanted pharmacological activity (e.g. in a manner similar to that described by Rivera et al (Nature Med; 1996; 2; 1028-1032).

A list of proteins/peptides whose interaction is disruptable using an agent such as a small molecule is given in Table 3. These disputable protein-protein interactions (PPI) may be used in the CAR system of the present invention. Further information on these PPIs is available from White et al 2008 (Expert Rev. Mol. Med. 10:e8).

TABLE 3

| Interacting Protein 1 | Interacting Protein 2 | Inhibitor of PPI |
|---|---|---|
| p53 | MDM2 | Nutlin |
| Anti-apoptotic Bcl2 | Apoptotic Bcl2 member | GX015and ABT-737 |
| member | | |
| Caspase-3, -7 | X-linked inhibitor of | DIABLO and DIABLO |
| or -9 | apoptosis protein (XIAP) | mimetics |
| RAS | RAF | Furano-indene derivative |
| FR2-7 | PD2 domain of DVL | FJ9 |
| T-cell factor (TCF) | Cyclic AMP response | ICG-001 |
| | element binding protein | |
| | (CBP) | |

Second binding domains which competitively bind to the same first binding domain as the agents described above, and thus may be used to co-localise the receptor component and signalling component of the signalling system in the absence of the agent, may be identified using techniques and methods which are well known in the art. For example such second binding domains may be identified by display of a single domain VHH library.

The first binding domain and/or second binding domain of the signalling system may comprise a variant(s) which is able to specifically bind to the reciprocal binding domain and thus facilitate co-localisation of the receptor component and signalling component.

Variant sequences may have at least 80%, 85%, 90%, 95%, 98% or 99% sequence identity to the wild-type sequence, provided that the sequences provide an effective dimerization system. That is, provided that the sequences facilitate sufficient co-localisation of the receptor and signalling components, in the absence of the agent, for productive signalling to occur upon binding of the antigen-binding domain to antigen.

The present invention also relates to a method for inhibiting a tunable CAR system, which method comprises the step of administering the agent. As described above, administration of the agent results in a disruption of the co-localization between the receptor component and the signalling component, such that signalling through the signalling component is inhibited even upon binding of antigen to the antigen binding domain.

The first and second binding domains may facilitate signalling through the CAR system which is proportional to the concentration of the agent which is present. Thus, whilst the agent binds the first binding domain or the second binding domain with a higher affinity than binding affinity between the first and second binding domains, co-localization of the receptor and signalling components may not be completely ablated in the presence of low concentrations of the agent. For example, low concentrations of the agent may decrease the total level of signalling in response to antigen without completely inhibiting it. The specific concentrations of agent will differ depending on the level of signalling required and the specific binding domains and agent. Levels of signalling and the correlation with concentration of agent can be determined using methods known in the art, as described above.

Receptor Component

The receptor component may comprise an antigen-binding domain, an optional spacer domain, a transmembrane domain and a first biding domain. When expressed in a cell, the receptor component localises to the cell membrane. Here, the antigen-binding domain of the molecule is orientated on the extracellular side of the membrane and the first binding domain is localised to the intracellular side of the membrane.

The receptor component therefore provides the antigen-binding function of the CAR system of the present invention.

The tunable CAR of the cell of the present invention is or comprises a receptor component as defined herein.

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain of the CAR which binds to CD19 may be any domain which is capable of binding CD19. For example, the antigen binding domain may comprise a CD19 binder as described in Table 4.

The antigen binding domain of the CAR which binds to CD19 may comprise a sequence derived from one of the CD19 binders shown in Table 4.

TABLE 4

| Binder | References |
|---|---|
| HD63 | Pezzutto (Pezzutto, A. et al. J. Immunol. Baltim. |
| | Md 1950 138, 2793-2799 (1987) |
| 4g7 | Meeker et al (Meeker, T. C. et al. Hybridoma 3, |
| | 305-320 (1984) |
| Fmc63 | Nicholson et al (Nicholson, I. C. et al. Mol. |
| | Immunol. 34, 1157-1165 (1997) |
| B43 | Bejcek et al (Bejcek, B. E. et al. Cancer Res. |
| | 55, 2346-2351 (1995) |
| SJ25C1 | Bejcek et al (1995, as above) |
| BLY3 | Bejcek et al (1995, as above) |
| B4, or re-surfaced, | Roguska et al (Roguska, M. A. et al. Protein Eng. |
| or humanized B4 | 9, 895-904 (1996) |
| HB12b, | Kansas et al (Kansas, G. S. & Tedder, T. F. J. |
| optimized and | Immunol. Baltim. Md 1950 147, 4094-4102 (1991); |
| humanized | Yazawa et al (Yazawa et al Proc. Natl. Acad. |
| | Sci. U.S.A. 102, 15178-15183 (2005); |
| | Herbst et al (Herbst, R. et al. J. Pharmacol. |
| | Exp. Ther. 335, 213-222 (2010) |

The antigen binding domain of the CAR which binds to CD22 may be any domain which is capable of binding CD22. For example, the antigen binding domain may comprise a CD22 binder as described in Table 5.

TABLE 5

| Binder | References |
|---|---|
| M5/44 or | John et al (J. Immunol. Baltim. Md 1950 170, |
| humanized M5/44 | 3534-3543 (2003); and DiJoseph et al (Cancer |
| | Immunol. Immunother. CII 54, 11-24 (2005) |
| M6/13 | DiJoseph et al (as above) |
| HD39 | Dorken et al (J. Immunol. Baltim. Md 1950 |
| | 136, 4470-4479 (1986) |
| HD239 | Dorken et al (as above) |
| HD6 | Pezzutto et al (J. Immunol. Baltim. Md 1950 |
| | 138, 98-103 (1987) |
| RFB-4, or humanized | Campana et al (J. Immunol. Baltim. Md 1950 |
| RFB-4, or | 134, 1524-1530 (1985); Krauss et al |
| affinity matured | (Protein Eng. 16, 753-759 (2003), |
| | Kreitman et al (J. Clin. Oncol. Off. J. Am. |
| | Soc. Clin. Oncol. 30, 1822-1828 (2012)) |
| Tol5 | Mason et al (Blood 69, 836-840 (1987)) |
| 4KB128 | Mason et al (as above) |
| S-HCL1 | Schwarting et al (Blood 65, 974-983 (1985)) |
| mLL2 (EPB-2), | Shih et al (Int. J. Cancer J. Int. Cancer 56, |
| or humanized mLL2 - | 538-545 (1994)), Leonard et al (J. Clin. |
| hLL2 | Oncol. Off. J. Am. Soc. Clin. Oncol. 21, |
| | 3051-3059 (2003)) |
| M971 | Xiao et al (mAbs 1, 297-303 (2009)) |
| BC-8 | Engel et al (J. Exp. Med. 181, 1581-1586 |
| | (1995)) |
| HB22-12 | Engel et al (as above) |

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the cell of the present invention, the first and second CARs may comprise different spacer molecules. For example, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

The spacer for the anti-CD19 CAR may comprise a CD8 stalk spacer, or a spacer having a length equivalent to a CD8 stalk spacer. The spacer for the anti-CD19 CAR may have at least 30 amino acids or at least 40 amino acids. It may have between 35-55 amino acids, for example between 40-50 amino acids. It may have about 46 amino acids.

The spacer for the anti-CD22 CAR may comprise an IgG1 hinge spacer, or a spacer having a length equivalent to an IgG1 hinge spacer. The spacer for the anti-CD22 CAR may have fewer than 30 amino acids or fewer than 25 amino acids. It may have between 15-25 amino acids, for example between 18-22 amino acids. It may have about 20 amino acids.

Examples of amino acid sequences for these spacers are given below:

(hinge-CH2CH3 of human IgG1)

SEQ ID No. 38

AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):

SEQ ID No. 39

TTTPAPRPPTPAPTIASQPLSLRPEACRPAAGGAVHTRGLDFACDI
(human IgG1 hinge):

SEQ ID No. 40

AEPKSPDKTHTCPPCPKDPK
(CD2 ectodomain)

SEQ ID No. 41

KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
(CD34 ectodomain)

SEQ ID No. 42

SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

Since CARs are typically homodimers (see FIG. 1a), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. The spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

In the CD19/CD22 OR gate of the present invention the CD19CAR and/or the CD22 CAR may comprise a coiled coil spacer domain. In particular, the present invention provides a CD19/CD22 OR gate in which the CD19 CAR is tunable and dimeric and the CD22 CAR is multimeric and comprises an integral T-cell signalling endodomain. In this arrangement the CD19 CAR has a non-coiled coil spacer and the CD22 CAR has a coiled-coil spacer. The CD22 CAR may comprise a 41BB endodomain.

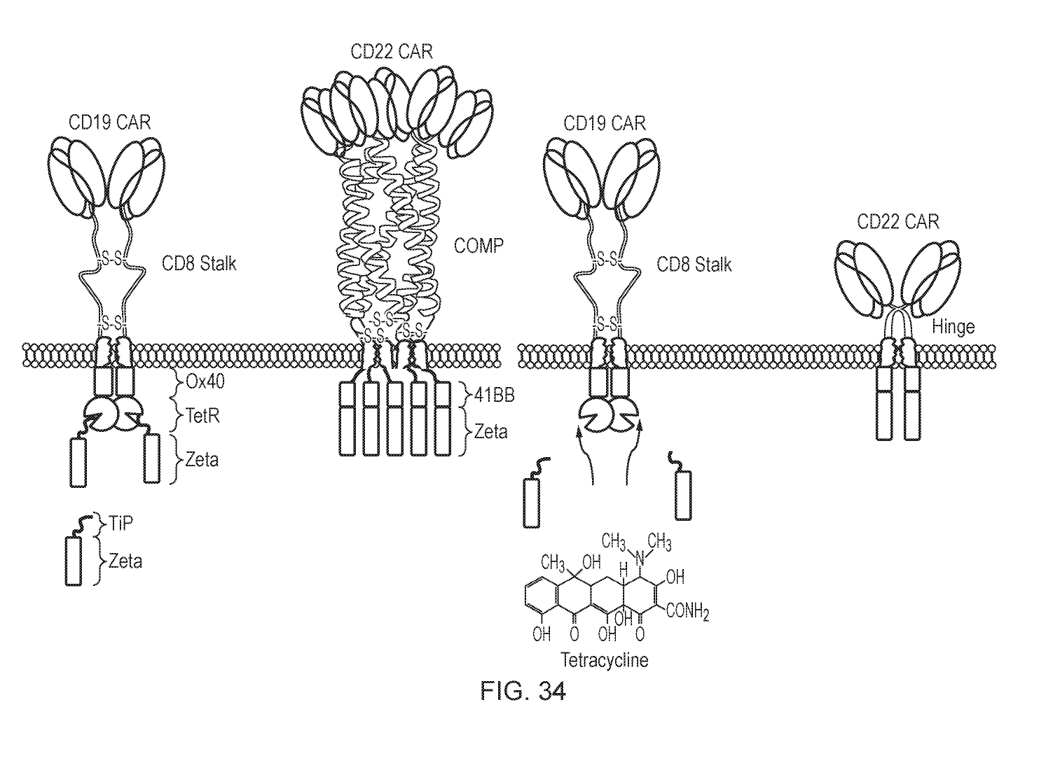

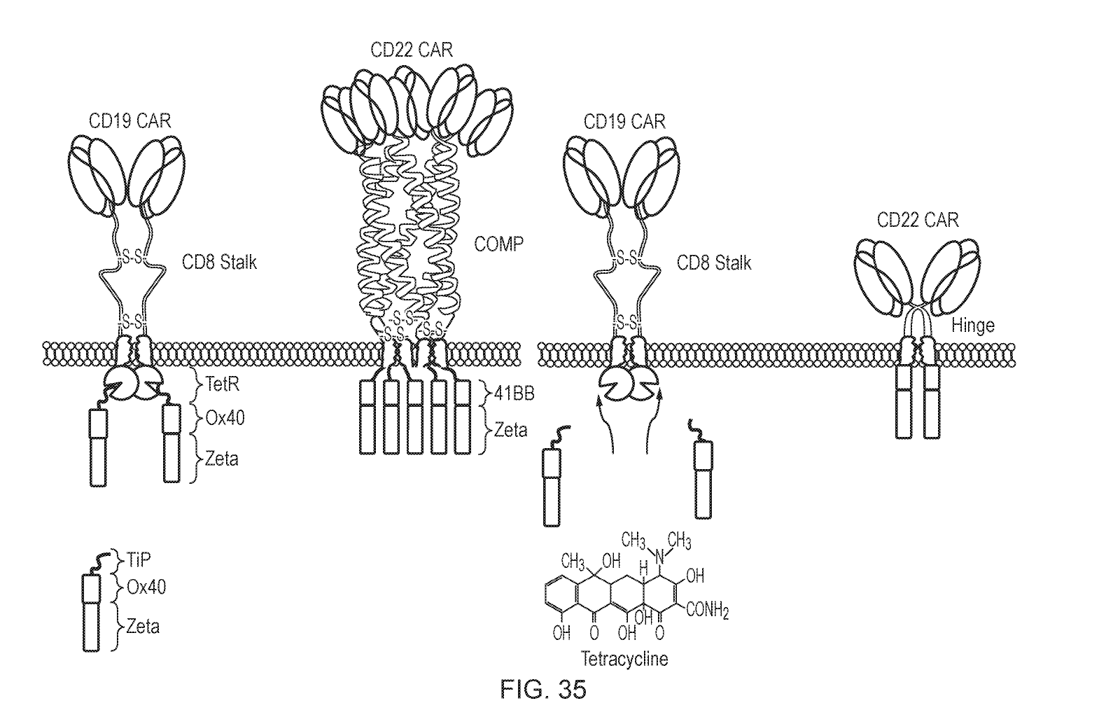

A coiled coil is a structural motif in which two to seven alpha-helices are wrapped together like the strands of a rope (FIGS. 34 and 35). Many endogenous proteins incorporate coiled coil domains. The coiled coil domain may be involved in protein folding (e.g. it interacts with several alpha helical motifs within the same protein chain) or responsible for protein-protein interaction. In the latter case, the coiled coil can initiate homo or hetero oligomer structures.

As used herein, the terms ‘multimer’ and ‘multimerization’ are synonymous and interchangeable with ‘oligomer’ and ‘oligomerization’.

The structure of coiled coil domains is well known in the art. For example as described by Lupas & Gruber (Advances in Protein Chemistry; 2007; 70; 37-38).

Coiled coils usually contain a repeated pattern, hxxhcxc, of hydrophobic (h) and charged (c) amino-acid residues, referred to as a heptad repeat. The positions in the heptad repeat are usually labeled abcdefg, where a and d are the hydrophobic positions, often being occupied by isoleucine, leucine, or valine. Folding a sequence with this repeating pattern into an alpha-helical secondary structure causes the hydrophobic residues to be presented as a ‘stripe’ that coils gently around the helix in left-handed fashion, forming an amphipathic structure. The most favourable way for two such helices to arrange themselves in the cytoplasm is to wrap the hydrophobic strands against each other sandwiched between the hydrophilic amino acids. Thus, it is the burial of hydrophobic surfaces that provides the thermodynamic driving force for the oligomerization. The packing in a coiled-coil interface is exceptionally tight, with almost complete van der Waals contact between the side-chains of the a and d residues.

The α-helices may be parallel or anti-parallel, and usually adopt a left-handed super-coil. Although disfavoured, a few right-handed coiled coils have also been observed in nature and in designed proteins.

The coiled coil domain may be any coiled coil domain which is capable of forming a coiled coil multimer such that a complex of CARs or accessory polypeptides comprising the coiled coil domain is formed.

The relationship between the sequence and the final folded structure of a coiled coil domain are well understood in the art (Mahrenholz et al; Molecular & Cellular Proteomics; 2011; 10(5):M110.004994). As such the coiled coil domain may be a synthetically generated coiled coil domain.

Examples of proteins which contain a coiled coil domain include, but are not limited to, kinesin motor protein, hepatitis D delta antigen, archaeal box C/D sRNP core protein, cartilage-oligomeric matrix protein (COMP), mannose-binding protein A, coiled-coil serine-rich protein 1, polypeptide release factor 2, SNAP-25, SNARE, Lac repressor or apolipoprotein E.

The sequence of various coiled coil domains is shown below:

Kinesin motor protein: parallel homodimer

(SEQ ID No. 50)

MHAALSTEVVHLRQRTEELLRCNEQQAAELETCKEQLFQSNMERKELHNT
VMDLRGN
Hepatitis D delta antigen: parallel homodimer

(SEQ ID No. 51)

GREDILEQWVSGRKKLEELERDLRKLKKKIKKLEEDNPWLGNIKGIIGKY
Archaeal box C/D sRNP core protein:
anti-parallel heterodimer

(SEQ ID No. 52)

RYVVALVKALEEIDESINMLNEKLEDIRAVKESEITEKFEKKIRELRELR
RDVEREIEEVM
Mannose-binding protein A: parallel homotrimer

(SEQ ID No. 53)

AIEVKLANMEAEINTLKSKLELTNKLHAFSM
Coiled-coil serine-rich protein 1: parallel
homotrimer

(SEQ ID No. 54)

EWEALEKKLAALESKLQALEKKLEALEHG
Polypeptide release factor 2: anti-parallel
heterotrimer
Chain A:

(SEQ ID No. 55)

INPVNNRIQDLTERSDVLRGYLDY
Chain B:

(SEQ ID No. 56)

VVDTLDQMKQGLEDVSGLLELAVEADDEETFNEAVAELDALEEKLAQ
LEFR
SNAP-25 and SNARE: parallel heterotetramer
Chain A:

(SEQ ID No. 57)

IETRHSEIIKLENSIRELHDMFMDMAMLVESQGEMIDRIEYNVEHAV
DYVE
Chain B:

(SEQ ID No. 58)

ALSEIETRHSEIIKLENSIRELHDMFMDMAMLVESQGEMIDRIEYNV
EHAVDYVERAVSDTKKAVKY
Chain C:

(SEQ ID No. 59)

ELEEMQRRADQLADESLESTRRMLQLVEESKDAGIRTLVMLDEQGEQ
LERIEEGMDQINKDMKEAEKNL
Chain D:

(SEQ ID No. 60)

IETRHSEIIKLENSIRELHDMFMDMAMLVESQGEMIDRIEYNVEHAV
DYVE
Lac repressor: parallel homotetramer

(SEQ ID No. 61)

SPRALADSLMQLARQVSRLE
Apolipoprotein E: anti-parallel heterotetramer

(SEQ ID No. 62)

SGQRWELALGRFWDYLRWVQTLSEQVQEELLSSQVTQELRALMDETMKEL
KAYKSELEEQLTARLSKELQAAQARLGADMEDVCGRLVQYRGEVQAMLGQ
STEELRVRLASHLRKLRKRLLRDADDLQKRLAVYQA

The coiled coil domain is capable of oligomerization. In certain embodiments, the coiled coil domain may be capable of forming a trimer, a tetramer, a pentamer, a hexamer or a heptamer.

A coiled-coil domain is different from a leucine zipper. Leucine zippers are super-secondary structures that function as a dimerization domains. Their presence generates adhesion forces in parallel alpha helices. A single leucine zipper consists of multiple leucine residues at approximately 7-residue intervals, which forms an amphipathic alpha helix with a hydrophobic region running along one side. This hydrophobic region provides an area for dimerization, allowing the motifs to “zip” together. Leucine zippers are typically 20 to 40 amino acids in length, for example approximately 30 amino acids.

Leucine zippers are typically formed by two different sequences, for example an acidic leucine zipper heterodimerizes with a basic leucine zipper. An example of a leucine zipper is the docking domain (DDD1) and anchoring domain (AD1) which are described in more detail below.

Leucine zippers form dimers, whereas the coiled-coiled spacers of the present invention for multimers (trimers and above). Leucine zippers heterodimerise in the dimerization portion of the sequence, whereas coiled-coil domains homodimerise.

In one embodiment, the present invention provides a hyper-sensitive CAR.

The hyper-sensitive CAR is provided by increasing the valency of the CAR. In particular, the use of a coiled coil spacer domain which is capable of interacting to form a multimer comprising more than two coiled coil domains, and therefore more than two CARs, increases the sensitivity to targets expressing low density ligands due to increasing the number of ITAMs present and avidity of the oligomeric CAR complex.

One or both of the CAR(s) of the OR gate of the present invention, particularly the CD22 CAR, may comprise a coiled coil spacer domain which enables the multimerization of at least three CAR-forming polypeptides. In other words, the CAR comprises a coiled coil domain which is capable of forming a trimer, a tetramer, a pentamer, a hexamer or a heptamer of coiled coil domains.

Examples of coiled coil domains which are capable of forming multimers comprising more than two coiled coil domains include, but are not limited to, those from cartilage-oligomeric matrix protein (COMP), mannose-binding protein A, coiled-coil serine-rich protein 1, polypeptide release factor 2, SNAP-25, SNARE, Lac repressor or apolipoprotein E (see SEQ ID Nos. 50-62 above).

The coiled coil domain may be the COMP coiled coil domain.

COMP is one of the most stable protein complexes in nature (stable from 0° C.-100° C. and a wide range of pH) and can only be denatured with 4-6M guanidine hydrochloride. The COMP coiled coil domain is capable of forming a pentamer. COMP is also an endogenously expressed protein that is naturally expressed in the extracellular space. This reduces the risk of immunogenicity compared to synthetic spacers. Furthermore, the crystal structure of the COMP coiled coil motif has been solved which gives an accurate estimation on the spacer length. The COMP structure is ˜5.6 nm in length (compared to the hinge and CH2CH3 domains from human IgG which is ˜8.1 nm).

The coiled coil domain may consist of or comprise the sequence shown as SEQ ID No. 63 or a fragment thereof.

SEQ ID No. 63

DLGPQMLRELQETNAALQDVRELLRQQVREITFLKNTVMECDACG

It is possible to truncate the COMP coiled-coil domain at the N-terminus and retain surface expression. The coiled-coil domain may therefore comprise or consist of a truncated version of SEQ ID No. 63, which is truncated at the N-terminus. The truncated COMP may comprise the 5 C-terminal amino acids of SEQ ID No. 63, i.e. the sequence CDACG (SEQ ID No. 64). The truncated COMP may comprise 5 to 44 amino acids, for example, at least 5, 10, 15, 20, 25, 30, 35 or 40 amino acids. The truncated COMP may correspond to the C-terminus of SEQ ID No. 63. For example a truncated COMP comprising 20 amino acids may comprise the sequences QQVREITFLKNTVMECDACG (SEQ ID No. 65). Truncated COMP may retain the cysteine residue(s) involved in multimerisation. Truncated COMP may retain the capacity to form multimers.

Various coiled coil domains are known which form hexamers such as gp41 dervived from HIV, and an artificial protein designed hexamer coiled coil described by N. Zaccai et al. (2011) Nature Chem. Bio., (7) 935-941). A mutant form of the GCN4-p1 leucine zipper forms a heptameric coiled-coil structure (J. Liu. et al., (2006) PNAS (103) 15457-15462).

The coiled coil domain may comprise a variant of one of the coiled coil domains described above, providing that the variant sequence retains the capacity to form a coiled coil oligomer. For example, the coiled coil domain may comprise a variant of the sequence shown as SEQ ID No. 50 to 63 having at least 80, 85, 90, 95, 98 or 99% sequence identity, providing that the variant sequence retains the capacity to form a coiled coil oligomer.

The percentage identity between two polypeptide sequences may be readily determined by programs such as BLAST which is freely available at http://blast.ncbi.nlm.nih.qov.

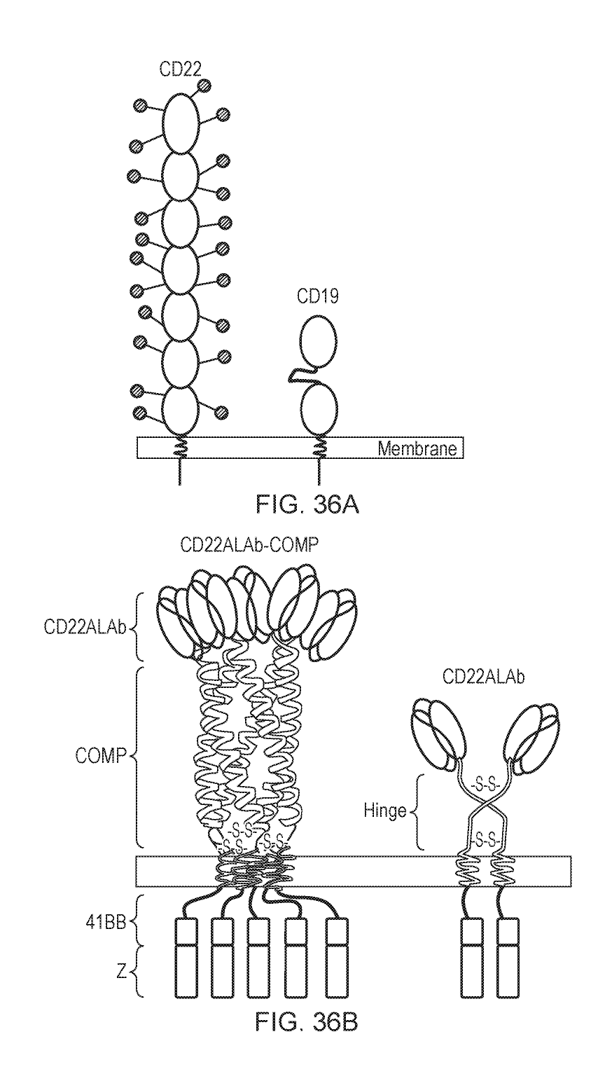

The present inventors have also found that, for an anti-CD22 CAR comprising a CD22ALAb antigen binding domain, the presence of a coiled-coil spacer domain leads to more efficient target cell killing than a “regular” spacer domain (see FIGS. 36 to 38 and Example 10).

Thus, the present invention also provides the aspects outlined in the following numbered paragraphs:

1. A chimeric antigen receptor (CAR) comprising a CD22-binding domain which comprises
a) a heavy chain variable region (VH) having complementarity determining regions (CDRs) with the following sequences:

CDR1

(SEQ ID No. 15)

NYWIN;
CDR2

(SEQ ID No. 16)

NIYPSDSFTNYNQKFKD
CDR3

(SEQ ID No. 17)

DTQERSWYFDV;

and
b) a light chain variable region (VL) having CDRs with the following sequences:

CDR1

(SEQ ID No. 18)

RSSQSLVHSNGNTYLH;
CDR2

(SEQ ID No. 19)

KVSNRFS
CDR3

(SEQ ID No. 20)

SQSTHVPWT;

and a coiled-coil spacer domain.
2. A CAR according to paragraph 2, wherein the CD22 binding domain comprises a VH domain having the sequence shown as SEQ ID No. 21, or SEQ ID NO 22; or a VL domain having the sequence shown as SEQ ID No 23, or SEQ ID No. 24 or a variant thereof having at least 90% sequence identity which retains the capacity to bind CD22.
3. A CAR according to paragraph 3, wherein the CD22 binding domain comprises the sequence shown as SEQ ID No 25 or SEQ ID No. 26 or a variant thereof having at least 90% sequence identity which retains the capacity to bind CD22.
4. A CAR according to any preceding paragraph, wherein the coiled-coil spacer domain enables the multimerization of at least three CAR-forming polypeptides.
5. A CAR according to paragraph 4, wherein the coiled-coil spacer domain is from: cartilage-oligomeric matrix protein (COMP), mannose-binding protein A, coiled-coil serine-rich protein 1, polypeptide release factor 2, SNAP-25, SNARE, Lac repressor or apolipoprotein E.
6. A CAR according to paragraph 5, wherein the coiled-coil spacer domain comprises one of the sequences shown as SEQ ID No. 50 to 63 or a fragment thereof or a variant thereof which has at least 80% sequence identity.
7 A cell comprising a CAR according to any preceding paragraph.
8. A nucleic acid encoding a CAR according to any preceding paragraph
9. A vector comprising a nucleic acid according to paragraph 8.
10. A pharmaceutical composition comprising a plurality of cells according to paragraph 7.
11. A method of treating a disease which comprises the step of administering a pharmaceutical composition according to paragraph 10 to a subject.

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues.

The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

The transmembrane domain may be derived from human Tyrp-1. The tyrp-1 transmembrane sequence is shown as SEQ ID No. 43.

SEQ ID No. 43

IIAIAVVGALLLVALIFGTASYLI

Receptor Component Comprising a Plurality of First Binding Domains

The tunable CAR/receptor component may comprise a plurality of first binding domains and thus be capable of recruiting more than one signalling component.

The plurality of first binding domains may be present in a single intracellular domain of the receptor component.

The receptor component may comprise an appropriate number of transmembrane domains such that each first binding domain is orientated on the intracellular side of the cell membrane. For example the receptor component may comprise 3, 5, 7, 9, 11, or more transmembrane domains. In this way, a single receptor component may recruit multiple signalling components amplifying signalling in response to antigen.

The first binding domains may each be variants which have a different affinity for the second binding domain of the signalling component.

Tunable CD19 and CD22 CARS

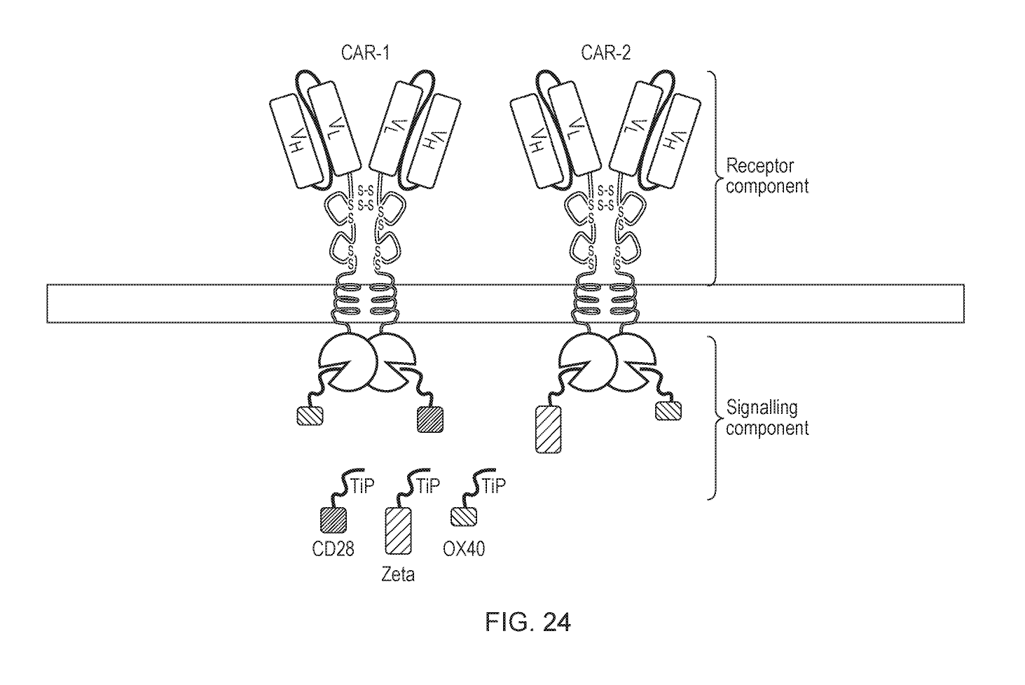

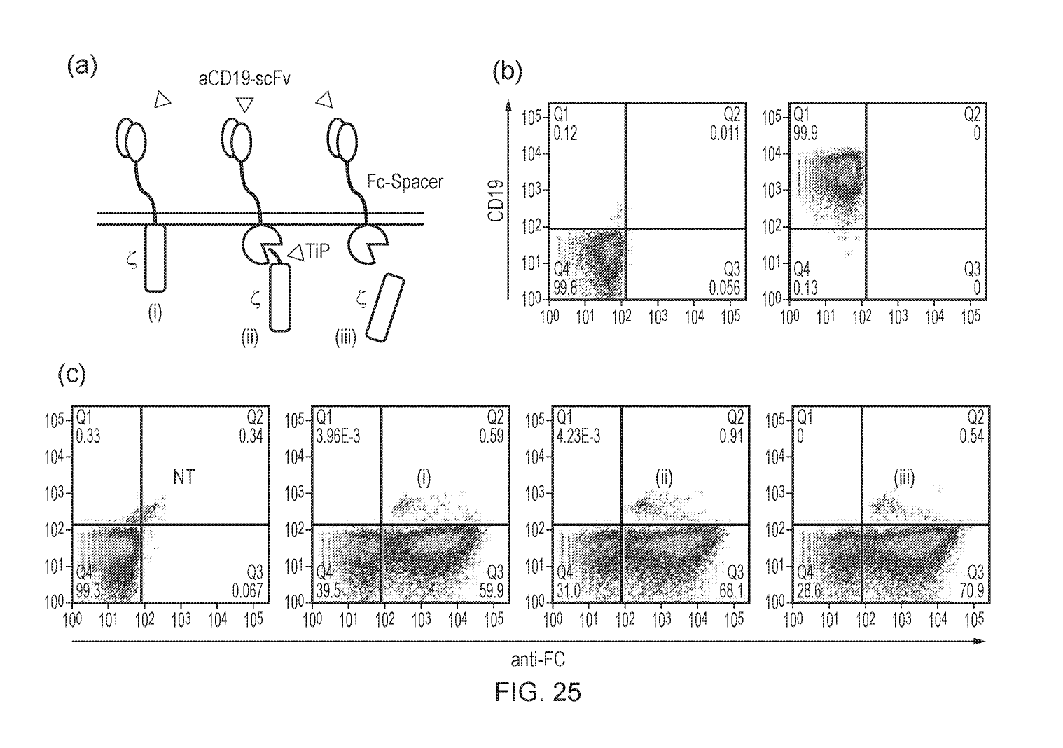

In one embodiment of the invention, both the CD19 and CD22 CARs are tunable. In this respect the two tunable CARs may be capable of binding the same intracellular signalling molecule, of they may be capable of binding different intracellular signalling molecule.

Where the two tunable CARs bind the same intracellular signalling molecule, heterodimerization of both the CD19 CAR and the CD22 CAR with the intracellular signalling molecule may be impaired or blocked by the same agent. The first binding domains of the CD19 and CD22 CARs may differ in residues which dictate their affinity for the second binding domain of the signalling component. In this way, a CAR system can be tuned such that signalling in response to one antigen is greater or lesser than the response to another (FIG. 24).

Methods suitable for altering the amino acid residues of the first or second binding domain such that the binding affinity between the two domains is altered are known in the art and include substitution, addition and removal of amino acids using both targeted and random mutagenesis. Methods for determining the binding affinity between a first binding domain and a second binding domain are also well known in the art and include bioinformatics prediction of protein-protein interactions, affinity electrophoresis, surface plasma resonance, bio-layer interferometry, dual polarisation interferometry, static light scattering and dynamic light scattering.

Where the two tunable CARs bind to a different intracellular signalling molecule, heterodimerization of the CD19 CAR and the CD22 CAR with their respective intracellular signalling molecules may be impaired or blocked by two different agents. This enables signalling via one CAR to be downregulated or stopped, without affecting signalling via the other CAR.

Signalling Component

The signalling component comprises a signalling domain and a second binding domain. The signalling component is a soluble molecule and thus localises to the cytoplasm when it is expressed in a cell, for example a T cell.

No signalling occurs through the signalling domain of the signalling component unless it is co-localised with a receptor component. Such co-localisation occurs only in the absence of the agent, as described above.

The intracellular signalling molecule defined in connection with the cell of the first aspect of the invention is or comprises an intracellular signalling component as defined herein.

Intracellular Signalling Domain

The intracellular signalling domain is the signal-transmission portion of a classical CAR. In the signalling system of tunable CAR the intracellular signalling domain (signalling domain) is located in the intracellular signalling molecule. In the absence of the agent, the membrane-bound, receptor component (i.e. tunable CAR) and the intracellular signalling component are brought into proximity. After antigen recognition, receptors cluster, native CD45 and CD148 are excluded from the synapse and a signal is transmitted to the cell.

As such the signalling domain of the signalling component is analogous to the endodomain of a classical CAR molecule.

The most commonly used signalling domain component is that of CD3-zeta endodomain, which contains 3 ITAMs and has the sequence shown as SEQ ID No. 44. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 1B).

CD3 Z endodomain

SEQ ID NO: 44

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

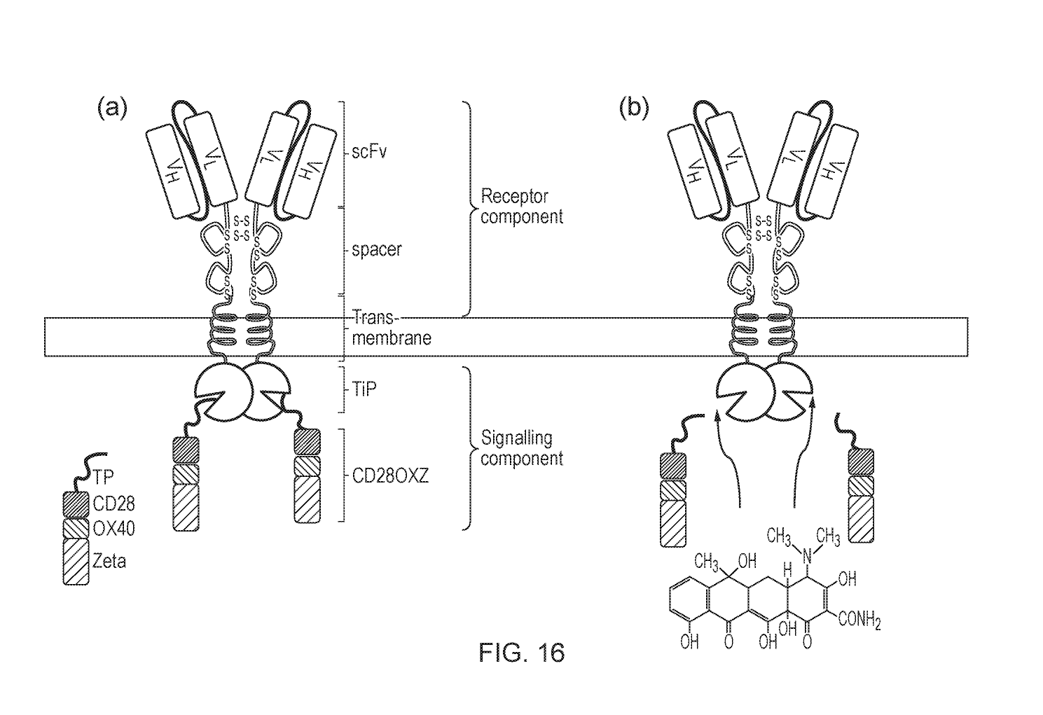

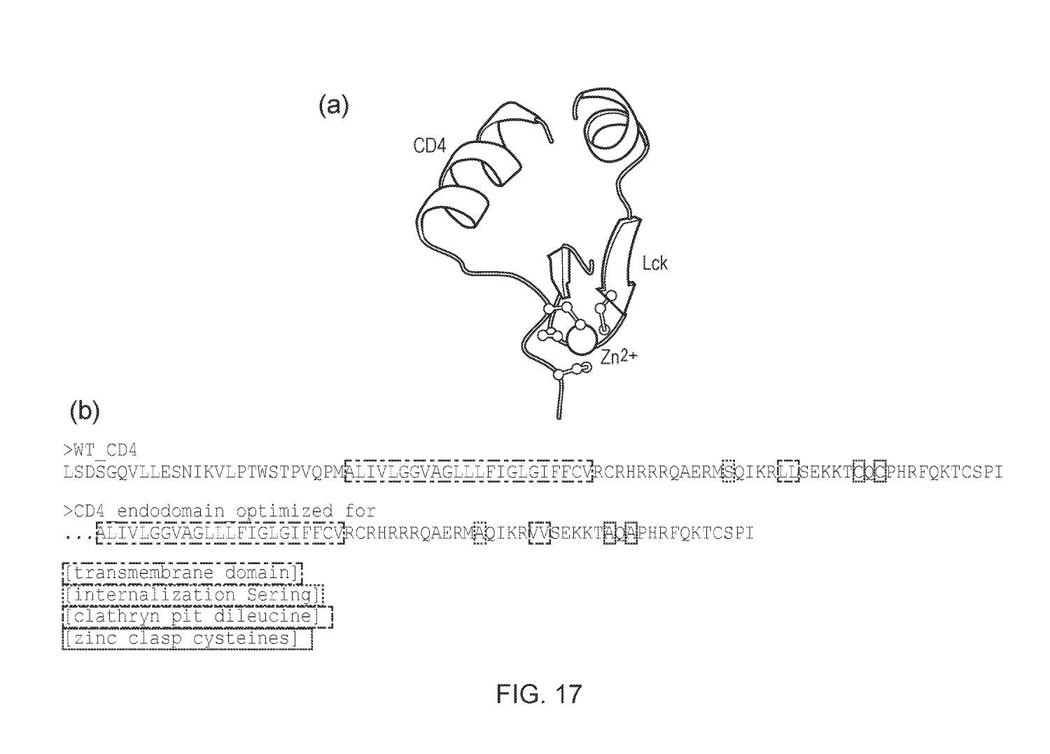

The signalling component (or classical CAR) may comprise the CD3-Zeta endodomain alone, the CD3-Zeta endodomain with that of either CD28 or OX40 or the CD28 endodomain and OX40 and CD3-Zeta endodomain (FIG. 16A).

The signalling component of a tunable CAR system and/or the intracellular T-cell signalling domain (endodomain) of a classical CAR may comprise the sequence shown as SEQ ID No. 45, 46 or 47 or a variant thereof having at least 80% sequence identity.

SEQ ID No. 45 comprising CD28 transmembrane
domain and CD3 Z endodomain
FWVLVVVGGVLACYSLLVTVAFIIFWVRRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID No. 46 comprising CD28 transmembrane domain
and CD28 and CD3 Zeta endodomains
FWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
SEQ ID No. 47 comprising CD28 transmembrane domain
and CD28, OX40 and CD3 Zeta endodomains.
FWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHST
LAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR

A variant sequence may have at least 80%, 85%, 90%, 95%, 98% or 99% sequence identity to SEQ ID No. 44, 45, 46 or 47, provided that the sequence provides an effective trans-membrane domain and an effective intracellular T cell signaling domain.

In a preferred arrangement, the intracellular domain of the tunable CAR comprises one or more co-stimulatory domains (for example CD28-OX40, OX40-CD28, CD28-41BB, or 41BB-CD28) and the intracellular signalling molecule comprises CD3zeta only.

Multiple Signalling Components

The cell may comprise a plurality of intracellular signalling molecules, each comprising a signalling domain and a second binding domain, wherein each second binding domain is bound by the same first binding domain of the receptor component but the signalling domains comprise different endodomains (FIG. 22). In this way, multiple different endodomains can be activated simultaneously. This is advantageous over a compound signalling domain since each signalling domain remains unencumbered from other signalling domains.

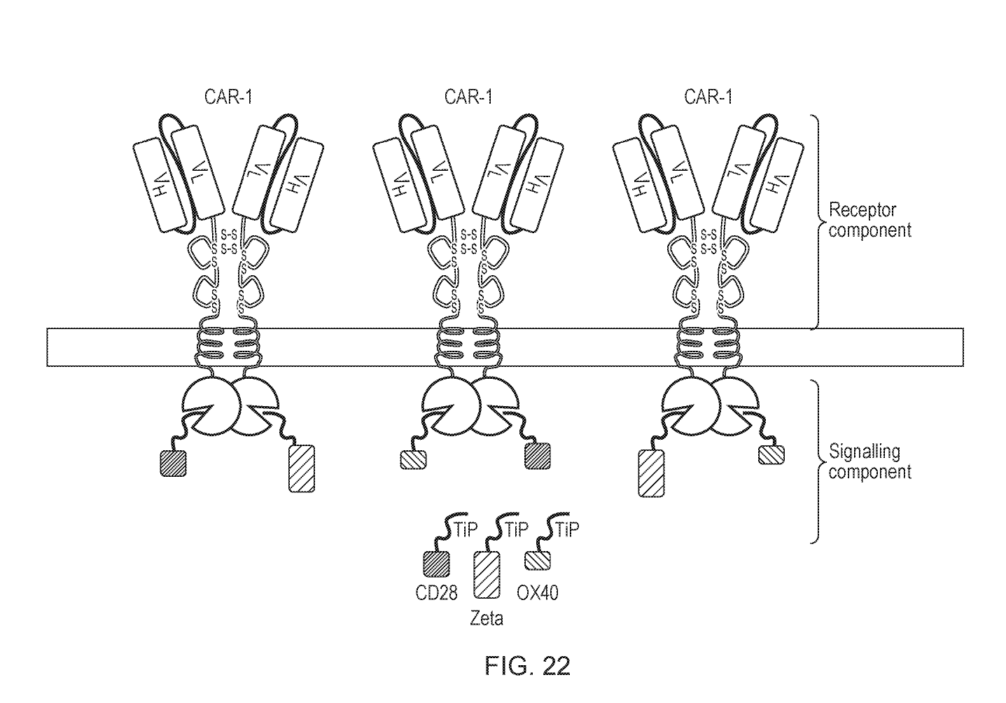

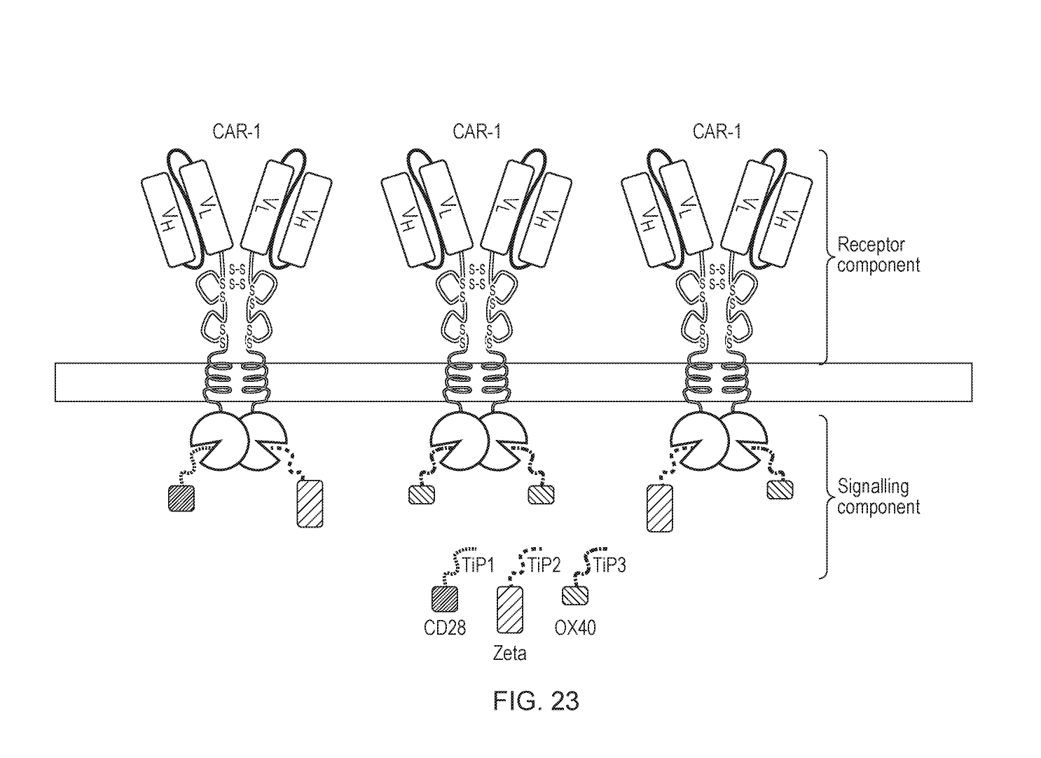

If each signalling component comprises a second binding domain which differs in residues which alter their affinity to the first binding domain of the receptor component, the signalling components comprising different signalling domains ligate to the first binding domain with differing kinetics (FIG. 23). This allows greater control over the signalling in response to antigen-binding by the receptor component as different signalling components are recruited to the receptor component in varying kinetics/dynamics. This is advantageous since rather than a fixed equal ratio of signal transmitted by a compound endodomain, an optimal T-cell activation signal may require different proportions of different immunological signals.

Nucleic Acid

The present invention further provides a nucleic acid encoding the first and/or second CAR(s) as defined herein.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

The present invention also provides a nucleic acid construct which comprises two or more of the following nucleic acid sequences:
(i) a nucleic acid sequence encoding a first CAR which binds CD19;
(ii) a nucleic acid sequence encoding a second CAR which binds CD22; and
(iii) a nucleic acid sequence encoding an intracellular signalling molecule.

The nucleic acid may produce a polypeptide which comprises the first CAR and/or second CAR and/or intracellular signalling molecule joined by a cleavage site. The cleavage site may be self-cleaving, such that when the polypeptide is produced, it is immediately cleaved into the various components, without the need for any external cleavage activity.

Various self-cleaving sites are known, including the Foot-and-Mouth disease virus (FMDV) 2A peptide and similar sequence (Donnelly et al, Journal of General Virology (2001), 82, 1027-1041), for instance like the 2A-like sequence from Thosea asigna virus which has the sequence shown as SEQ ID No. 48:

SEQ ID No. 48

RAEGRGSLLTCGDVEENPGP.

The co-expressing sequence may alternatively be an internal ribosome entry sequence (IRES) or an internal promoter.

The nucleic acid construct may have one of the following structures:

a) AgB1-spacer1-TM1-HD1-coexpr-AgB2-spacer2-TM2-endo2;
b) AgB1-spacer1-TM1-endo1-coexpr-AgB2-spacer2-TM2-HD2;
c) AgB2-spacer2-TM2-HD2-coexpr-AgB1-spacer1-TM1-endo1;
d) AgB2-spacer2-TM2-endo2-coexpr-AgB1-spacer1-TM1-HD1;
e) AgB1-spacer1-TM1-HD1-coexpr-AgB2-spacer2-TM1-HD1;
f) AgB2-spacer2-TM2-HD2-coexpr-AgB1-spacer1-TM1-HD1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
HD1 is a nucleic acid sequence encoding a heterodimerisation domain of the first CAR;
Endo1 is a nucleic acid sequence encoding an intracellular domain which comprises a signalling domain of the first CAR;
coexpr is a nucleic acid sequence enabling co-expression of both CARs;
AgB2 is a nucleic acid sequence encoding the antigen-binding domain of the second CAR;
spacer 2 is a nucleic acid sequence encoding the spacer of the second CAR;
TM2 is a a nucleic acid sequence encoding the transmembrane domain of the second CAR;
HD2 is a nucleic acid sequence encoding a heterodimerisation domain of the second CAR;
Endo2 is a nucleic acid sequence encoding an intracellular domain which comprises a signalling domain of the second CAR.

Where the nucleic acid construct encodes a tunable CD19 CAR and also encodes an intracellular signalling molecule, it may have one of the following structures:

a) AgB1-spacer1-TM1-HD1-coexpr1-AgB2-spacer2-TM2-endo2-coexpr2-HDICM-endoICM;
b) AgB1-spacer1-TM1-HD1-coexpr1-HDICM-endoICM-coexpr2-AgB2-spacer2-TM2-endo2;
c) AgB2-spacer2-TM2-endo2-coexpr1-AgB1-spacer1-TM1-HD1-coexpr2-HDICM-endoICM;
d) AgB2-spacer2-TM2-endo2-coexpr1-HDICM-endoICM-coexpr2-AgB1-spacer1-TM1-HD1;
e) AgB1-spacer1-TM1-HD1-coexpr1-AgB2-spacer2-TM1-HD1-coexpr2-HDICM-endoICM;
f) AgB1-spacer1-TM1-HD1-coexpr1-HDICM-endoICM-coexpr2-AgB2-spacer2-TM1-HD1;
g) AgB2-spacer2-TM2-HD2-coexpr1-AgB1-spacer1-TM1-HD1-coexpr2-HDICM-endoICM
h) AgB2-spacer2-TM2-HD2-coexpr1-HDICM-endoICM-coexpr2-AgB1-spacer1-TM1-HD1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
HD1 is a nucleic acid sequence encoding a heterodimerisation domain of the first CAR;
Endo1 is a nucleic acid sequence encoding an intracellular domain which comprises a signalling domain of the first CAR;
Coexpr1 is a nucleic acid sequence encoding a first co-expression site;
Coexpr2 is a nucleic acid sequence encoding a second co-expression site;
AgB2 is a nucleic acid sequence encoding the antigen-binding domain of the second CAR;
spacer 2 is a nucleic acid sequence encoding the spacer of the second CAR;
TM2 is a a nucleic acid sequence encoding the transmembrane domain of the second CAR;
HD2 is a nucleic acid sequence encoding a heterodimerisation domain of the second CAR;
Endo2 is a nucleic acid sequence encoding an intracellular domain which comprises a signalling domain of the second CAR;
HDICM is a nucleic acid sequence encoding a heterodimerization domain of the intracellular signalling molecule; and
endoICM is a nucleic acid sequence encoding a heterodimerization domain of the intracellular signalling molecule.

Alternative codons may be used in regions of sequence encoding the same or similar amino acid sequences, in order to avoid homologous recombination.

Due to the degeneracy of the genetic code, it is possible to use alternative codons which encode the same amino acid sequence. For example, the codons “ccg” and “cca” both encode the amino acid proline, so using “ccg” may be exchanged for “cca” without affecting the amino acid in this position in the sequence of the translated protein.

The alternative RNA codons which may be used to encode each amino acid are summarised in Table 6.

TABLE 6

| | U | C | A | G |
|---|---|---|---|---|
| U | 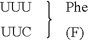 | 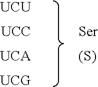 | 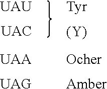 | 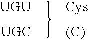 |
| | 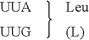 | | | 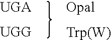 |
| C | 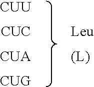 | 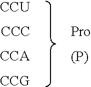 | 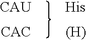 | 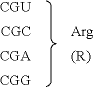 |
| | | | 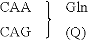 | |
| A | 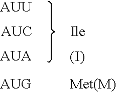 | 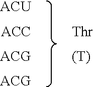 | 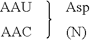 | 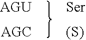 |
| | | | 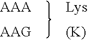 | 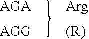 |
| G | 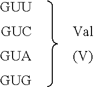 | 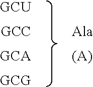 | 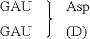 | 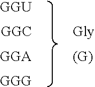 |
| | | | 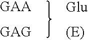 | |

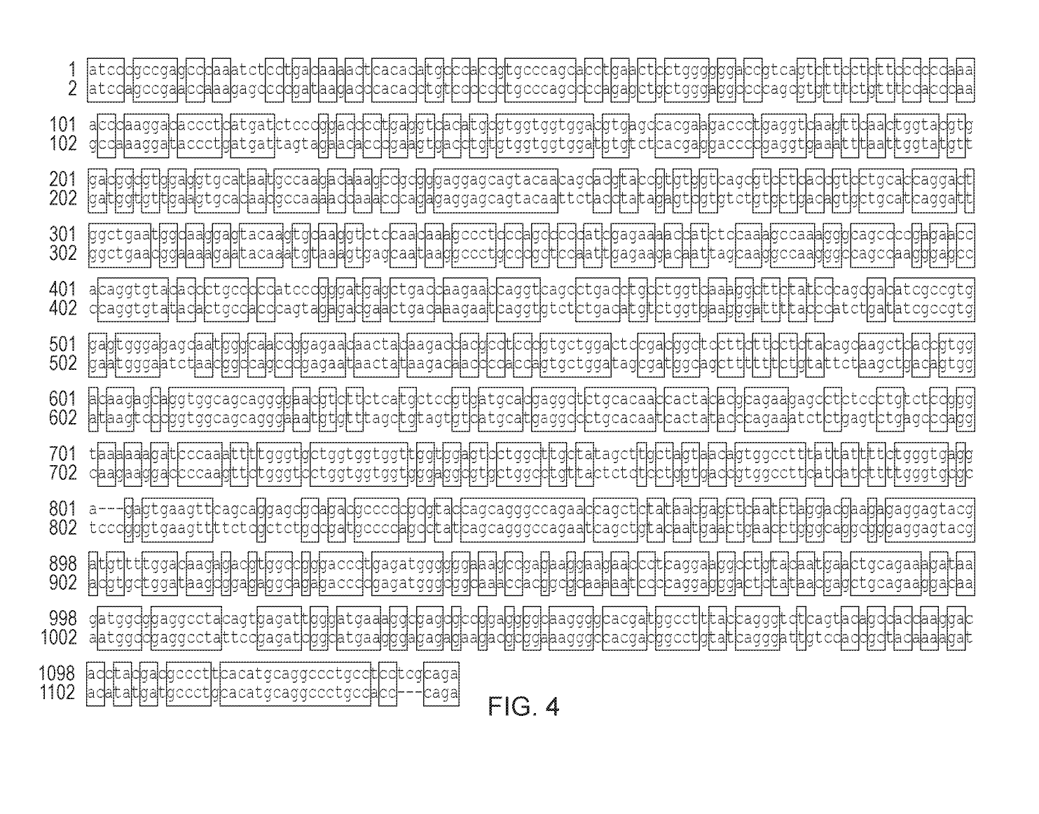

Alternative codons may be used in the portions of nucleic acid sequence which encode the spacer of the first CAR and the spacer of the second CAR, especially if the same or similar spacers are used in the first and second CARs. FIG. 4 shows two sequences encoding the spacer HCH2CH3—hinge, in one of which alternative codons have been used.

Alternative codons may be used in the portions of nucleic acid sequence which encode the transmembrane domain of the first CAR and the transmembrane of the second CAR, especially if the same or similar transmembrane domains are used in the first and second CARs. FIG. 4 shows two sequences encoding the CD28 transmembrane domain, in one of which alternative codons have been used.

Alternative codons may be used in the portions of nucleic acid sequence which encode all or part of the endodomain of the first or second CAR and all or part of the endodomain of the or each intracellular signalling molecule. Alternative codons may be used in the CD3ζ zeta endodomain. FIG. 4 shows two sequences encoding the CD3ζ zeta endodomain, in one of which alternative codons have been used.

Alternative codons may be used in one or more co-stimulatory domains, such as the CD28 endodomain.

Alternative codons may be used in one or more domains which transmit survival signals, such as OX40 and 41BB endodomains.

Alternative codons may be used in the portions of nucleic acid sequence encoding a CD3zeta endodomain and/or the portions of nucleic acid sequence encoding one or more costimulatory domain(s) and/or the portions of nucleic acid sequence encoding one or more domain(s) which transmit survival signals.

Cell

The present invention relates to a cell which co-expresses a first CAR and a second CAR at the cell surface, wherein one CAR binds CD19 and the other CAR binds CD22.

The cell may be any eukaryotic cell capable of expressing a CAR at the cell surface, such as an immunological cell.

In particular the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR—expressing cells, such as CAR-expressing T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

The present invention also provide a cell composition comprising CAR expressing T cells and/or CAR expressing NK cells according to the present invention. The cell composition may be made by transducing a blood-sample ex vivo with a nucleic acid according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells are generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3ζ monoclonal antibody.

A CAR T cell of the invention may be made by:
(i) isolation of a T cell-containing sample from a subject or other sources listed above; and
(ii) transduction or transfection of the T cells with one or more nucleic acid sequence(s) encoding the first and second CAR.

The T cells may then by purified, for example, selected on the basis of co-expression of the first and second CAR.

Vector

The present invention also provides a vector, or kit of vectors which comprises one or more CAR-encoding and/or intracellular signal molecule-encoding nucleic acid sequence(s). Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

Pharmaceutical Composition

The present invention also relates to a pharmaceutical composition containing a plurality of CAR-expressing cells, such as T cells or NK cells according to the first aspect of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The cells of the present invention are capable of killing cancer cells, such as B-cell lymphoma cells. CAR—expressing cells, such as T cells, may either be created ex vivo either from a patient's own peripheral blood (1st party), or in the setting of a haematopoietic stem cell transplant from donor peripheral blood (2nd party), or peripheral blood from an unconnected donor (3rd party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

The cells of the present invention may be capable of killing target cells, such as cancer cells. The target cell is recognisable by expression of CD19 or CD22.

TABLE 7
expression of lymphoid antigens on lymphoid leukaemias

| | CD19 | CD22 | CD10 | CD7 | CD5 | CD3 | clg μ | slg μ |
|---|---|---|---|---|---|---|---|---|
| Early | 100 | >95 | 95 | 5 | 0 | 0 | 0 | 0 |
| pre-B | | | | | | | | |
| Pre-B | 100 | 100 | >95 | 0 | 0 | 0 | 100 | 0 |
| Transi- | 100 | 100 | 50 | 0 | 0 | 0 | 100 | 0 |
| tional | | | | | | | | |
| pre-B | | | | | | | | |
| B | 100 | 100 | 50 | 0 | 0 | 0 | >95 | >95 |
| T | <5 | 0 | 0 | 100 | 95 | 100 | 0 | 0 |

Taken from Campana et al. (Immunophenotyping of leukemia. J. Immunol. Methods 243, 59-75 (2000)). clg μ—cytoplasic Immunoglobulin heavy chain; slg μ—surface Immunoglobulin heavy chain.

The expression of commonly studied lymphoid antigens on different types of B-cell leukaemias closely mirrors that of B-cell ontogeny (see FIG. 2).

The T cells of the present invention may be used to treat cancer, in particular B-cell malignancies.

Examples of cancers which express CD19 or CD22 are B-cell lymphomas, including Hodgkin's lymphoma and non-Hodgkins lymphoma; and B-cell leukaemias.

For example the B-cell lymphoma may be Diffuse large B cell lymphoma (DLBCL), Follicular lymphoma, Marginal zone lymphoma (MZL) or Mucosa-Associated Lymphatic Tissue lymphoma (MALT), Small cell lymphocytic lymphoma (overlaps with Chronic lymphocytic leukemia), Mantle cell lymphoma (MCL), Burkitt lymphoma, Primary mediastinal (thymic) large B-cell lymphoma, Lymphoplasmacytic lymphoma (may manifest as Waldenström macroglobulinemia), Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma or Primary central nervous system lymphoma.

The B-cell leukaemia may be acute lymphoblastic leukaemia, B-cell chronic lymphocytic leukaemia, B-cell prolymphocytic leukaemia, precursor B lymphoblastic leukaemia or hairy cell leukaemia.

The B-cell leukaemia may be acute lymphoblastic leukaemia.

Treatment with the T cells of the invention may help prevent the escape or release of tumour cells which often occurs with standard approaches. The methods provided by the present invention for treating a disease may involve monitoring the progression of the disease and any toxic activity and administering an agent suitable for use in the CAR system according to the first aspect of the invention to inhibit CAR signalling and thereby reduce or lessen any adverse toxic effects.

The methods provided by the present invention for treating a disease may involve monitoring the progression of the disease and monitoring any toxic activity and adjusting the dose of the agent administered to the subject to provide acceptable levels of disease progression and toxic activity.

Monitoring the progression of the disease means to assess the symptoms associated with the disease over time to determine if they are reducing/improving or increasing/worsening.

Toxic activities relate to adverse effects caused by the CAR cells of the invention following their administration to a subject. Toxic activities may include, for example, immunological toxicity such as cytokine release syndrome (CRS), neurotoxitity, biliary toxicity and/or respiratory distress syndrome.

The level of signalling through the or each tunable CAR, and therefore the level of activation of CAR cells expressing the CAR(s), may be adjusted by altering the amount of agent(s) present, or the amount of time the agent(s) is/are present. The level of CAR cell activation may be augmented by decreasing the dose of agent administered to the subject or decreasing the frequency of its administration. Conversely, the level of CAR cell activation may be reduced by increasing the dose of the agent, or the frequency of administration to the subject.

Higher levels of CAR cell activation are likely to be associated with reduced disease progression but increased toxic activities, whilst lower levels of CAR cell activation are likely to be associated with increased disease progression but reduced toxic activities.

The present invention also provides a method for treating and/or preventing a disease in a subject which subject comprises cells of the invention, which method comprises the step of administering an agent to the subject. As such, this method involves administering a suitable agent to a subject which already comprises CAR cells of the present invention.

The dose of agent administered to a subject, and/or the frequency of administration, may be altered in order to provide an acceptable level of both disease progression and toxic activity. The specific level of disease progression and toxic activities determined to be ‘acceptable’ will vary according to the specific circumstances and should be assessed on such a basis. The present invention provides a method for altering the activation level of the CAR cells in order to achieve this appropriate level.

The agent may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The present invention provides a CAR cell of the present invention for use in treating and/or preventing a disease.

The invention also relates to the use of a CAR cell of the present invention in the manufacture of a medicament for the treatment and/or prevention of a disease.

The present invention also provides an agent suitable for inhibiting a CAR system according to the first aspect of the invention for use in treating and/or preventing a disease.

The present invention also provides an agent for use in inhibiting a CAR system according to the first aspect of the invention in a CAR cell.

The invention also provides the use of an agent suitable for inhibiting a CAR system according to the first aspect of the invention in the manufacture of a medicament for the treatment and/or prevention of a disease.

The invention will now be further described by way of Examples, which are meant to serve to assist one of ordinary skill in the art in carrying out the invention and are not intended in any way to limit the scope of the invention.

### EXAMPLES

### Example 1—Proof-of-Concept of a CD19/CD22 Logical ‘OR’ Gate

A CD19 ‘OR’ CD22 CAR gate was constructed by co-expression of a CD19 and a CD22 CAR in the same vector. The anti-CD19 binder was a scFv derived from the re-surfaced B4 antibody (Roguska et al. (1996) Protein Eng. 9, 895-904), and the anti-CD22 binder was a scFv derived from the humanized RFB4 antibody. A human IgG1 hinge-CH2-CH3 spacer was used for both CARs, the coding sequence of which was codon-wobbled to avoid homologous recombination by the integrating vector. The TM domain in both CARs was derived from that of CD28, and both CAR endodomains comprised of CD3-Zeta. Once again, these homologous sequences were codon-wobbled. Co-expression was achieved by cloning the two CARs in frame separated by a FMD-2A peptide. The amino acid sequence of the CD19/CD22 ‘OR’ gate construct is shown as SEQ ID NO: 49.

SEQ ID NO: 49

MSLPVTALLLPLALLLHAARPYPYDVPDYASLSGGGGSQVQLVQSGAEVK
KPGASVKVSCKASGYTFTSNWMHWVRQAPGQGLEWMGEIDPSDSYTNYNQ
KFKGRVTITVDKSASTAYMELSSLRSEDTAVYYCARGSNPYYYAMDYWGQ
GTLVTVSSGGGGSGGGGSGGGGSEIVLTQSPATLSLSPGERATLSCSASS
GVNYMHWYQQKPGQAPRRWIYDTSKLASGVPARFSGSGSGTSYSLTISSL
EPEDFAVYYCHQRGSYTFGGGTKLEIKRSDPTTTPAPRPPTPAPTIASQP
LSLRPEACRPAAGGAVHTRGLDFACDIFWVLVVVGGVLACYSLLVTVAFI
IFWVRRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPRRAEGRGSLLTCGDVEENPGPMEFGLSWLFLVAI
LKGVQCEVQLVESGGGLVQPGGSLRLSCAASGFAFSIYDMSWVRQVPGKG
LEWVSYISSGGGTTYYPDTVKGRFTISRDNSRNTLDLQMNSLRVEDTAVY
YCARHSGYGSSYGVLFAYWGQGTLVTVSSGGGGSGGGGSGGGGSDIQMTQ
SPSSLSASVGDRVTITCRASQDISNYLNWLQQKPGKAPKLLIYYTSILHS
GVPSRFSGSGSGTEFTLTISSLQPEDFATYYCQQGNTLPWTFGQGTKLEI
KRSDPAEPKSPDKTHTCPPCPAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPGKKDPKFWVLVVVG
GVLACYSLLVTVAFIIFWVRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

To demonstrate co-expression of both CARs, the scFv of each CAR was tagged with an epitope tag (HA or V5 respectively). This subsequent single open-reading frame was cloned into the SFG retroviral vector. T-cells were transduced with this vector and both CARs could be detected on the T-cells surface expressing the cassette by staining with anti-HA and anti-V5 and studying expression by flow cytometry.

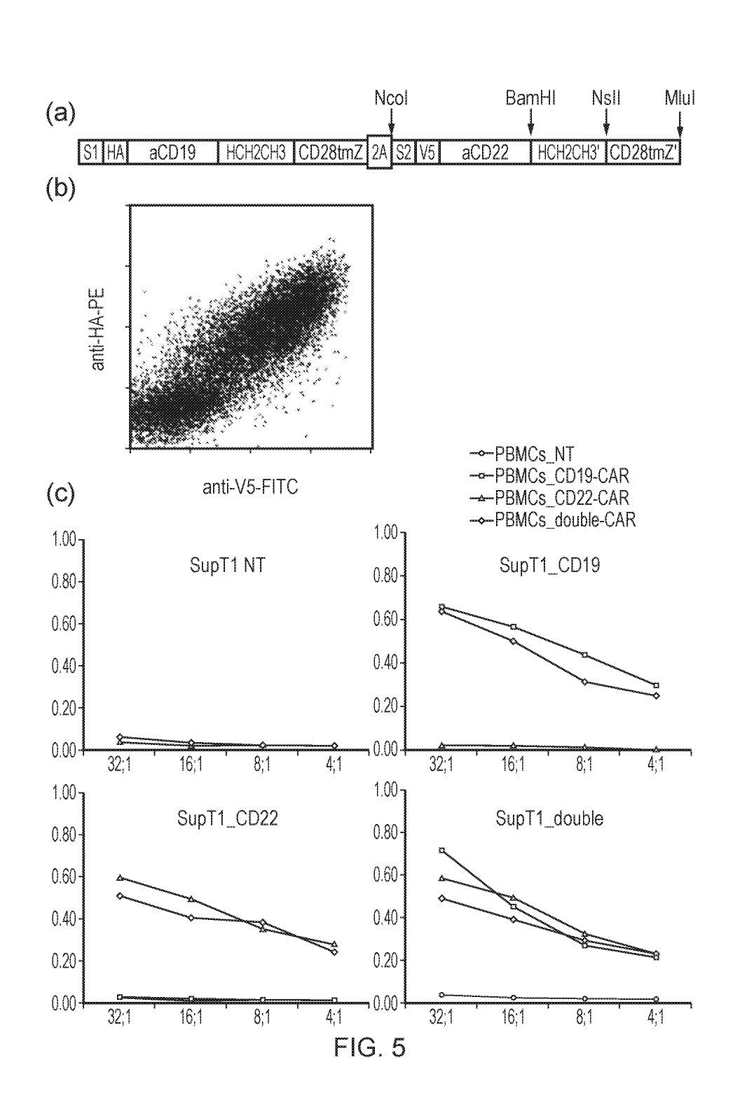

Next, T-cells expressing the CD19 OR CD22 CAR gate were challenged with target cells, expressing neither, both or one antigen along with control T-cells which expressed no CARs, or just anti-CD19 CAR alone, or anti-CD22 CAR alone. We found that the OR-gated CAR T-cells could kill target cells expressing either one or both target antigens (FIG. 5).

### Example 2—Identification and Characterisation of CD19ALAb and CD22ALAb

A CD19-binder (CD19ALAb) was identified, humanised and the binding affinities of both murine and humanised IgGs and scFvs were identified and compared with the “gold-standard” anti-CD19 binder, fmc63. In parallel, and a CD22-binder (CD22ALAb) was identified, humanised and the binding affinities of both murine and humanised IgGs and scFvs were identified and compared with the “gold-standard” anti-CD22 binder, M971.

Experiments were performed on a Biacore T200 instrument using HBS-P as running and dilution buffer. BIAevaluation software Version 2.0 was used for data processing. For binding kinetics, mouse anti-human IgG or goat anti-mouse IgG was covalently coupled to a CM5 Sensor Chip. IgG or scFv-Fc proteins were captured, and various concentrations of interaction partner protein injected over the flow cell at a flow rate of 30 μl/min. Kinetic rate constants were obtained by curve fitting according to a 1:1 Langmuir binding model. Bulk refractive index differences were subtracted using a blank control flow cell in which capture antibody had been immobilized to the same level as the active surface. A double reference subtraction was performed using buffer alone.

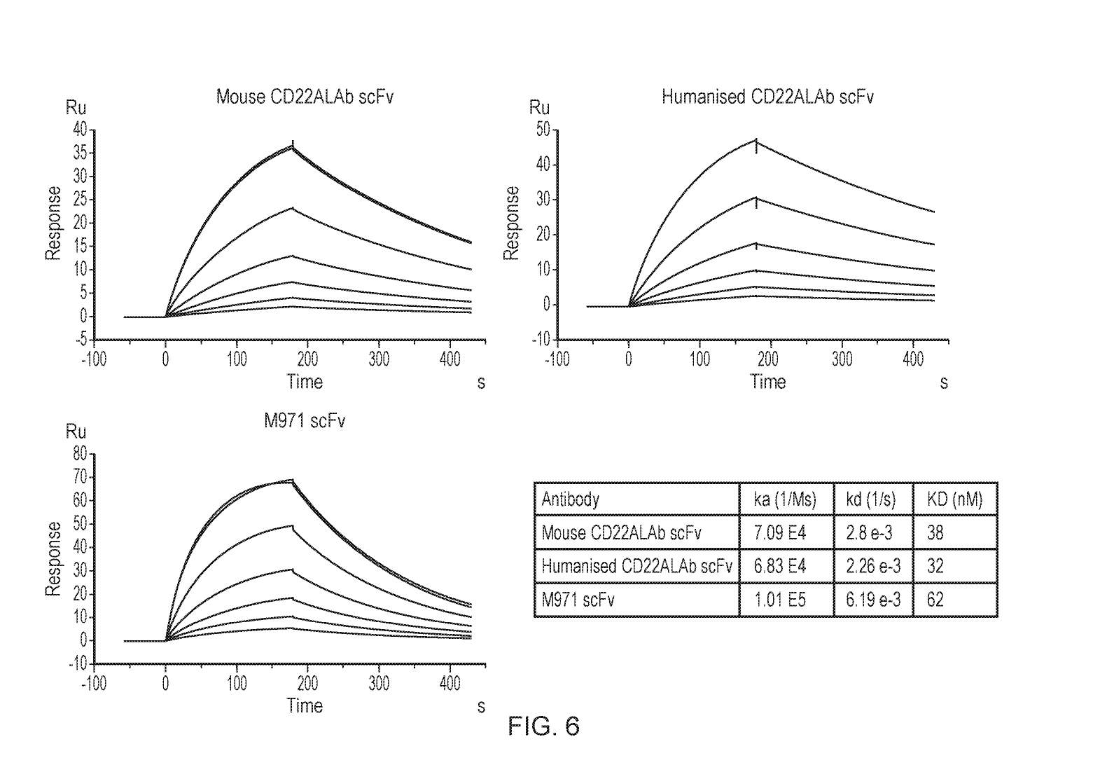

The results are shown in FIGS. 6 to 8.

The data show that humanised CD22ALAb has comparable binding affinity to CD22 to murine CD22ALAb (FIG. 6) and similar binding kinetics. Both murine and humanised CD22ALAb in an scFv format have significantly higher binding affinity to CD22 than the gold-standard CD22-binding antibody, M971 (FIG. 6).

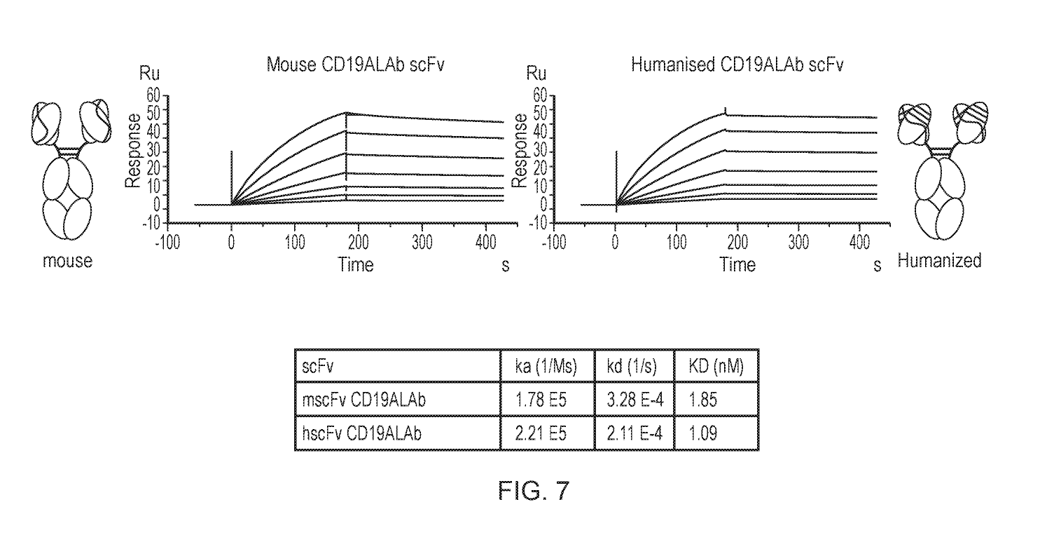

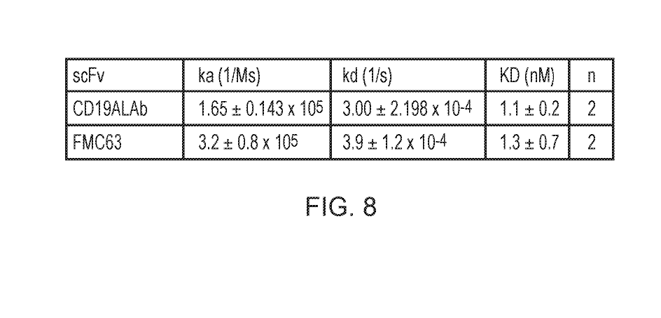

Although the binding affinity of murine and humanised CD19ALAb in an IgG format was found to be similar (data not shown), surprisingly the binding affinity of humanised CD19ALAb was found to be higher than murine CD19ALAb in an scFv format (FIG. 7). The binding affinity of CD19ALAb is comparable (possibly slightly better) than that of the gold-standard anti-CD19 Ab, fmc63 (FIG. 8).

### Example 3—Comparative Functional Assays with CD19ALAb/Fmc63 CARs and CD22ALAb/M971 CARs

The antigen binding domain of a CAR can affect its function. In this study, CARs were created comprising CD19ALAb and CD22ALAb and function was compared with an equivalent CAR having an antigen-binding domain based on fmc63 or M971.

CARs comprising scFvs based on fmc63 (anti-CD19) and M971 (anti-CD22) can be considered as the gold standard antibodies as both CARs are in clinical development.

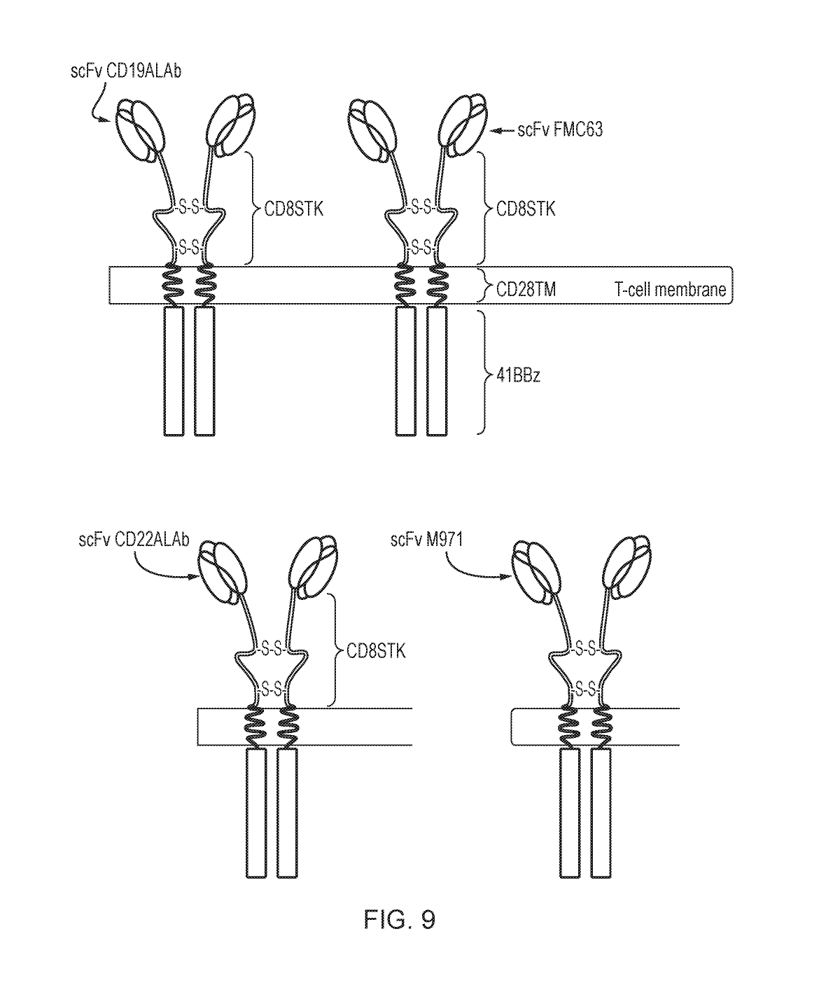

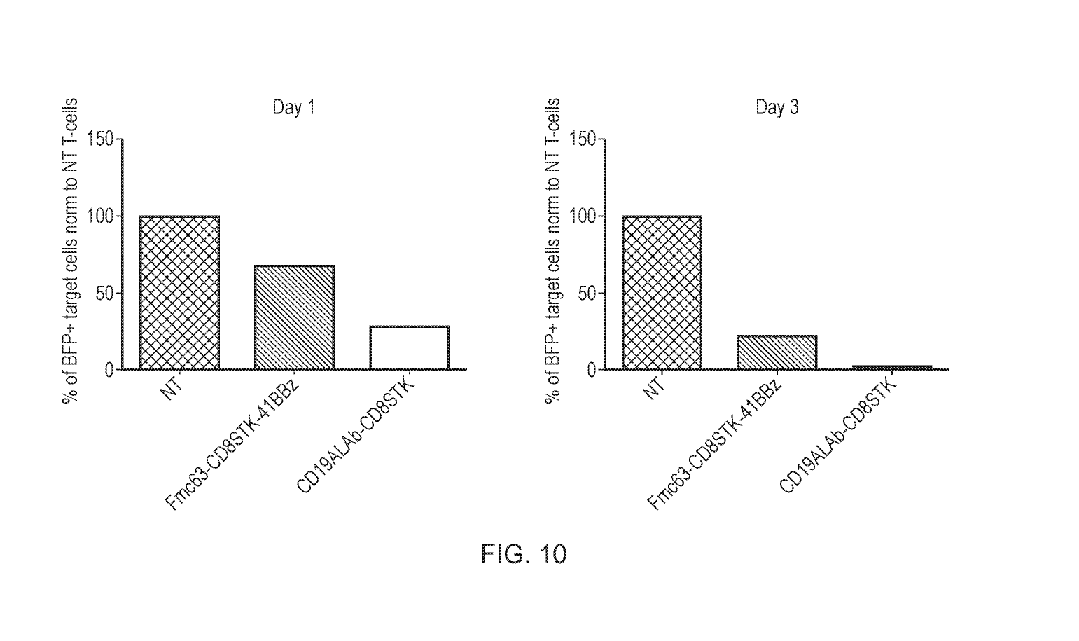

CARs were constructed and expressed based on CD19ALAb, fmc63, CD22ALAb and M971. Their structure is shown in FIG. 9. The CARs differed solely in their antigen binding domain. In all constructs, the binding domains were linked to the membrane with a CD8 stalk spacer and contained intracellular activatory motifs from 41BB and CD3-zeta.

Retroviruses were produced by transient transfection of 293T cells with plasmids encoding the CARs, gag/pol and the envelope protein RD114. After 3 days the supernatants were harvested and used to transduce PHA/IL2-activated PBMCs with equal titres of retrovirus on retronectin-coated plates. Six days post-transduction CAR-expression was confirmed by flow cytometry and PBMCs were co-cultured in a 1:1 ratio with either CD19+ BFP SupT1 cells (fmc63 and CD19ALAb CARs) or CD22+ BFP SupT1 cells (M971 and CD22ALAb CARs). Target cell killing was assayed after one and three days. Also after one and three days, supernatants were removed and interferon-y levels were assayed by ELISA.

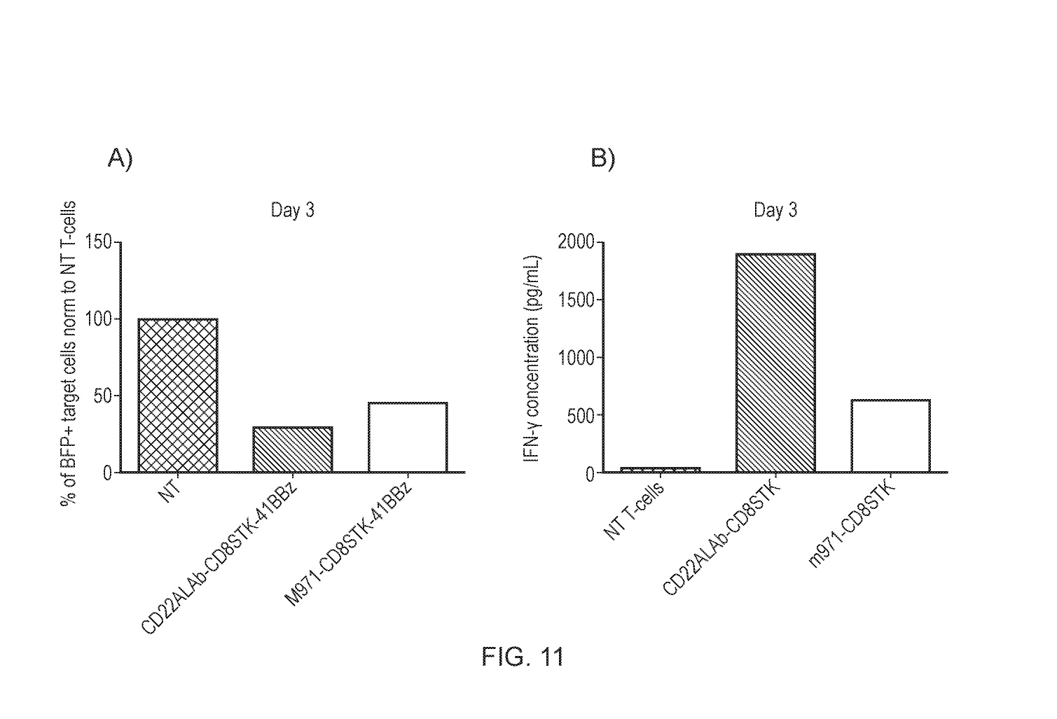

The results are shown in FIGS. 10 and 11.

As shown in FIG. 10, the CAR with a CD19ALAb antigen binding domain gave more killing of CD19+ve target cells (FIG. 10) at both Day1 and Day 3, than the equivalent CAR with a fmc63 binding domain.

With regard to CD22, the CAR with a CD22ALAb antigen binding domain gave more killing of CD22+ve target cells (FIG. 11a) after three days than the equivalent CAR with an M971 binding domain. IFNγ release was significantly higher with the CD22ALAb CAR than the M971 CAR after the same time frame.

CARs having an antigen-binding domain based on CD19ALAb and CD22ALAb therefore have improved properties in terms of target cell killing than equivalent CARs based on fmc63 and M971.

The CD22ALAb result is particularly surprising, given the findings reported in Haso et al (2013) as above. In that study, different anti-CD22 CARs were made and tested, with binding domains based on the anti-CD22 antibodies HA22, BL22 and m971. HA22 and BL22 scFvs bind to Ig domain 3 of CD22, whereas m971 binds within Ig domain 5-7 of CD22 (see Haso et al (2013) FIG. 2B). It was reported that the m971-derived CAR showed superior target cell killing activity than HA22-derived CAR, which finding is attributed to the importance of the CD22 epitope targeted by the CAR (Haso et al (2013) page 1168, last full paragraph). It is concluded that targeting a membrane proximal domain of CD22 is “the key element” in developing a highly active anti-CD22 CAR (Discussion, last paragraph). Contrary to this finding, the data shown here in FIG. 11 demonstrate that CD22ALAb, which targets an epitope in Ig domain 3 of CD22—a “membrane distal” epitope compared to the Ig domain 5-7 epitope targeted by m971—has superior target cell killing ability than an m971-based anti-CD22 CAR.

### Example 4—Investigating OR Gate Constructs with Different Endodomain Combinations

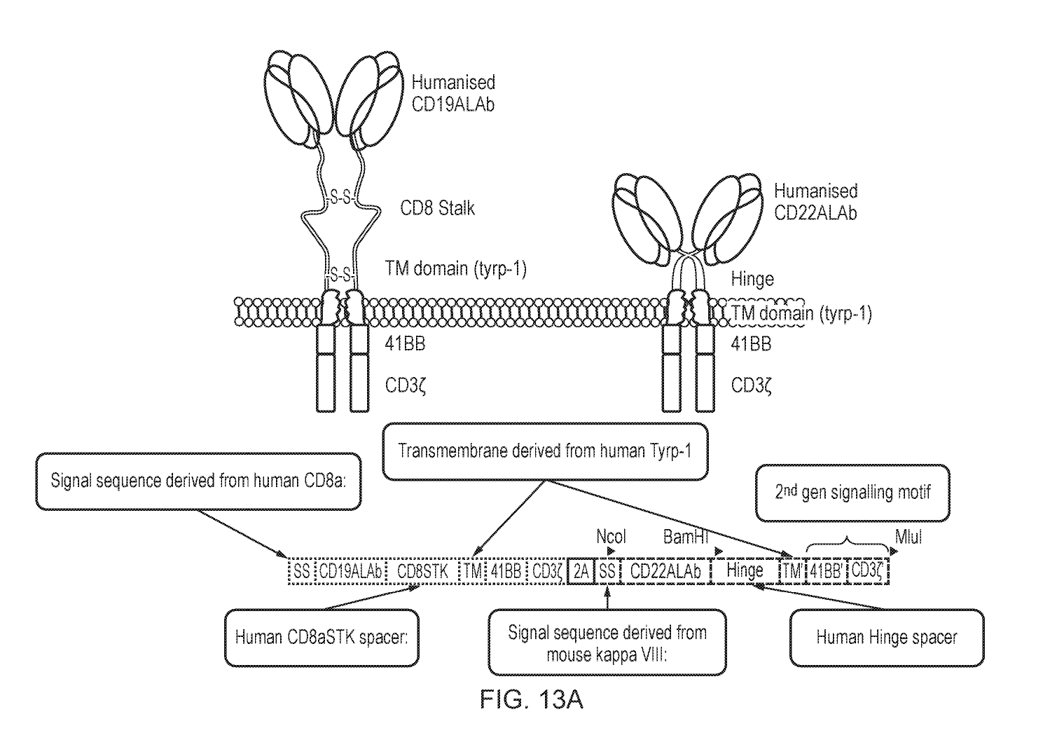

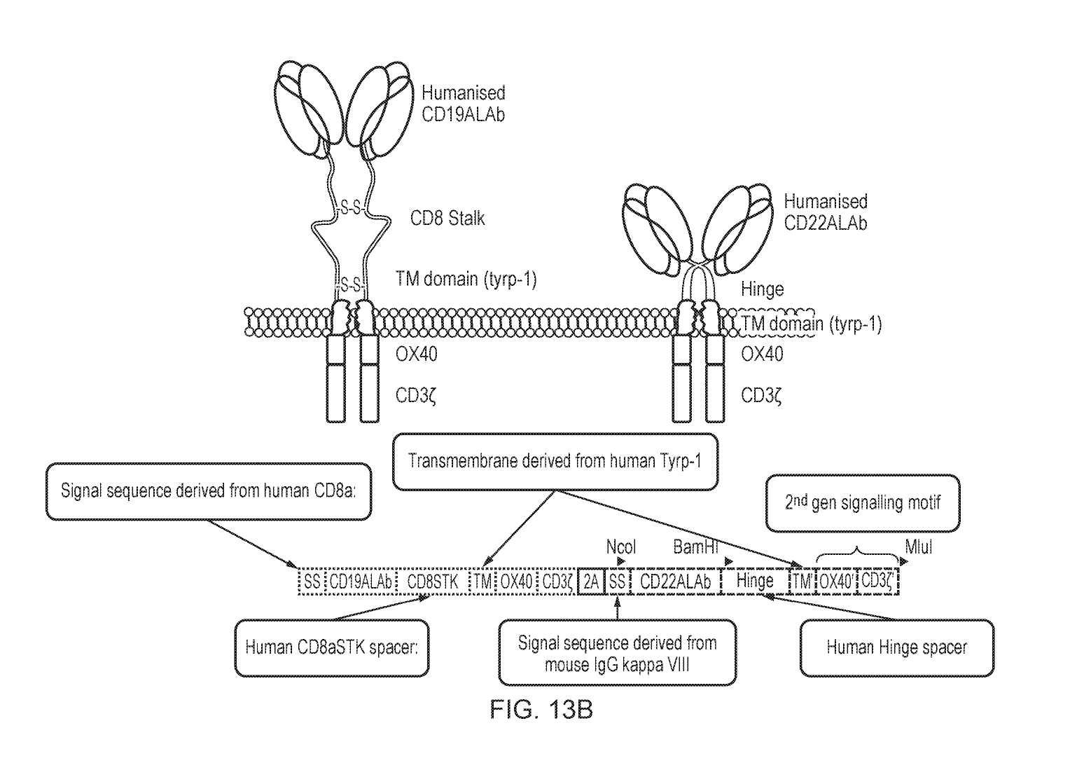

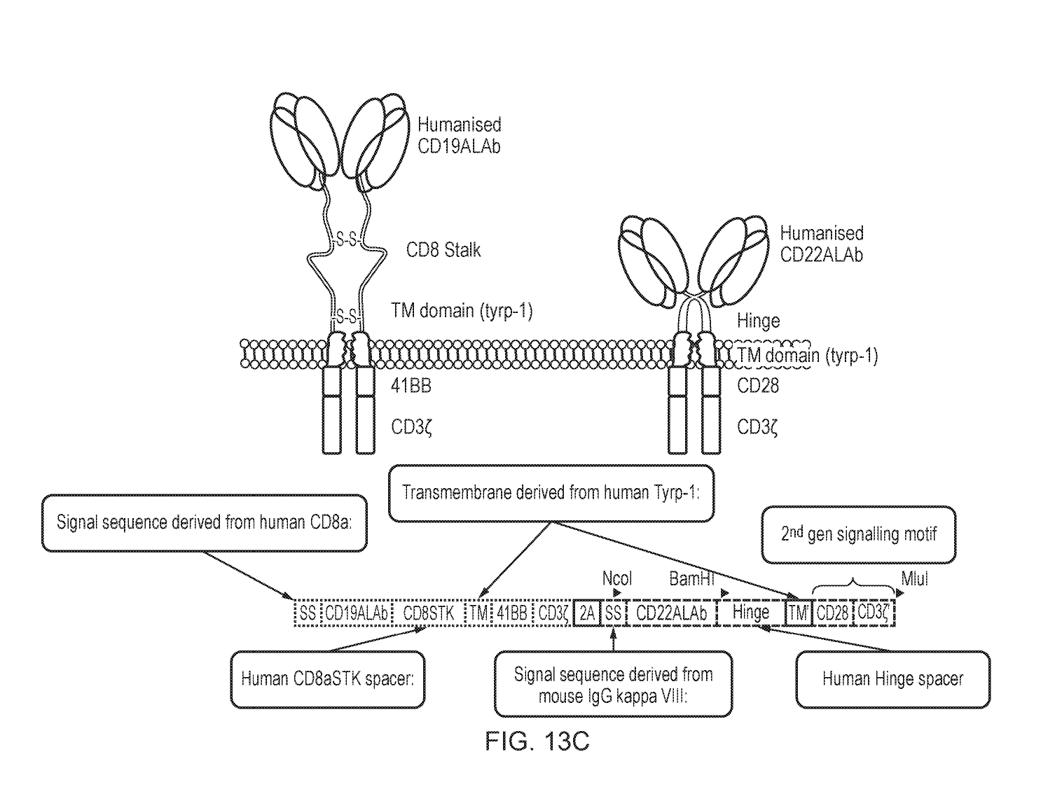

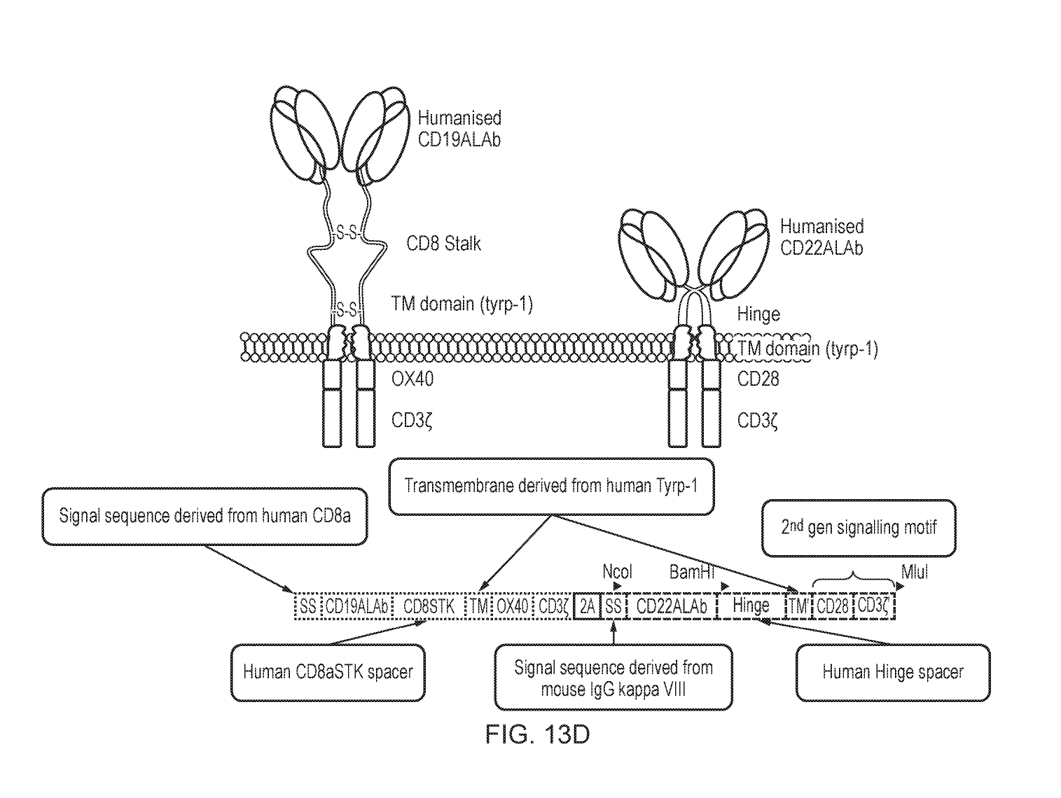

Four OR gate constructs were developed as shown in FIG. 13. They all encoded CD19/CD22 OR gates having identical antigen-binding domains, spacer domains and transmembrane domains: the only difference between the construct was in the endodomains, which were as shown in the following Table:

| Construct | CD19 CAR endodomain | CD22 CAR endodomain |
|---|---|---|
| A | 41BB-CD3ζ | 41BB-CD3ζ |
| B | OX40-CD3ζ | OX40-CD3ζ |
| C | 41BB-CD3ζ | CD28-CD3ζ |
| D | OX40-CD3ζ | CD28-CD3ζ |

The capacity of cells expressing each CD19/CD22 OR gate to kill Raji cells in vitro was assayed as described above. Transduced PBMCs expressing the various OR gate combinations were co-cultured for 72 hours with CD19+/CD22+ Raji target cells at both a 1:1 and 1:10 effector:target cell ratio.

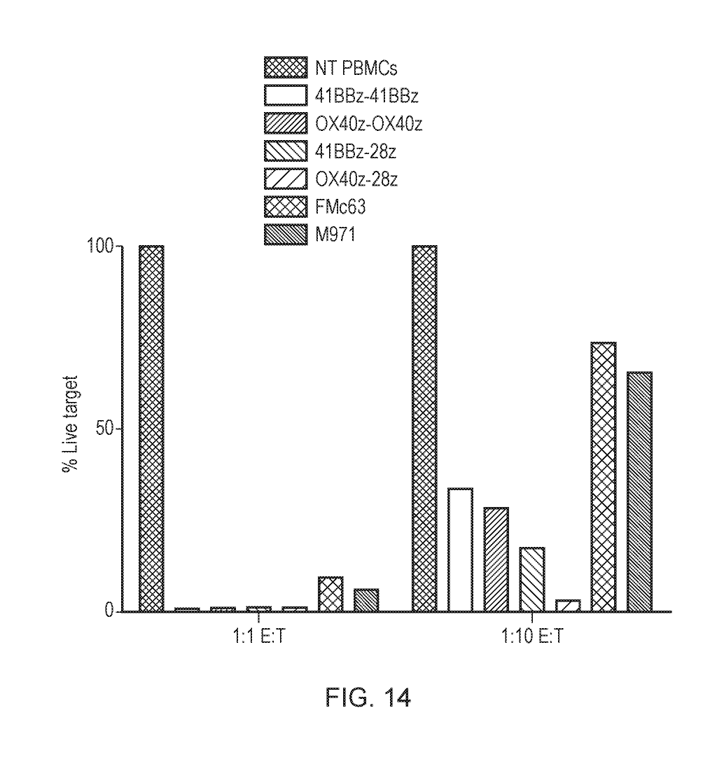

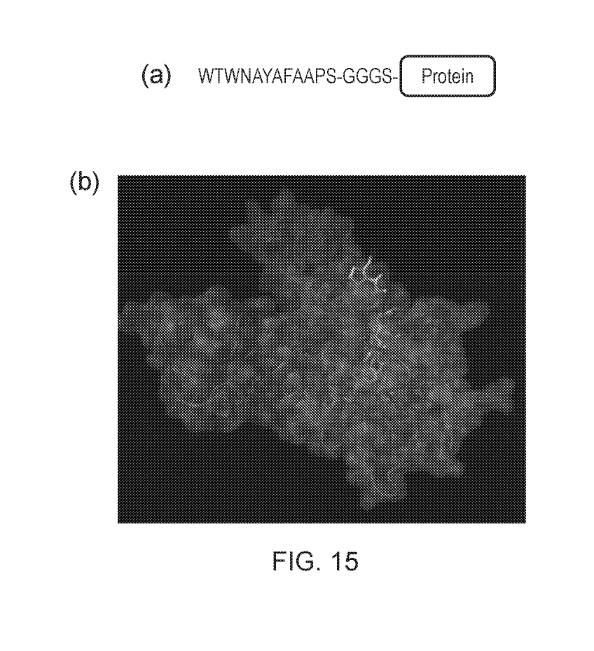

The results are shown in FIG. 14. All four OR gates were found to kill target cells significantly better than the fmc63 and M971 CARs. With the 1:10 effector:target cell ratio, it was shown that the “split” endodomain OR gates, which have 4-1BBzeta/OX40zeta on one CAR and CD28zeta on the other CAR, had the best killing activity.

### Example 5—Functionality of a Tunable Signalling System

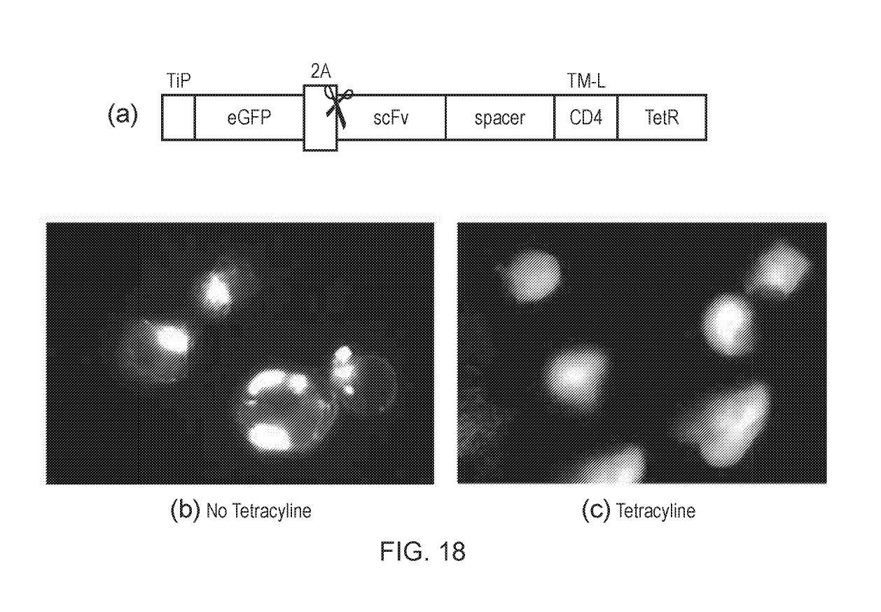

A bicistronic construct was expressed as a single transcript which self-cleaves at the 2A site to yield TiP fused to eGFP and a CAR with TetR as its endodomain (FIG. 18a).

Fluorescent microscopy of SupT1 cells expressing this construct in the absence of tetracycline demonstrated that eGFP fluorescence can clearly be seen at the cell membrane (FIG. 18b); whilst in the presence of tetracycline the eGFP was cytoplasmic (FIG. 18c). These data demonstrate that tetracycline has displaced TiP from the TetR CAR.

### Example 6—Signalling Through a Tunable System

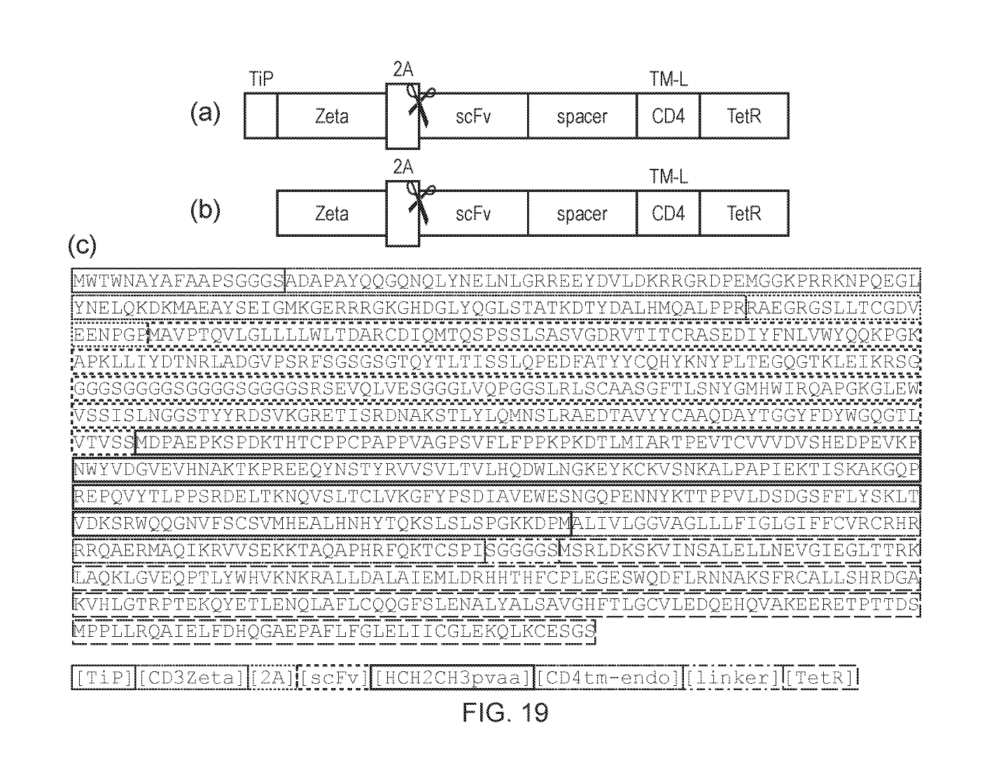

A bicistronic construct was expressed in BW5 T cells as a single transcript which self-cleaves at the 2A site to yield a signalling component which comprises TiP fused via a flexible linker to the endodomain of CD3-Zeta; and a receptor component which comprises a CD33 recognizing scFv, a spacer derived from the Fc domain of IgG1, a CD4 derived transmembrane and intracellular domain; and TetR (FIG. 19a). A control was also expressed which was identical except that TiP was absent from the signalling component (FIG. 19b).

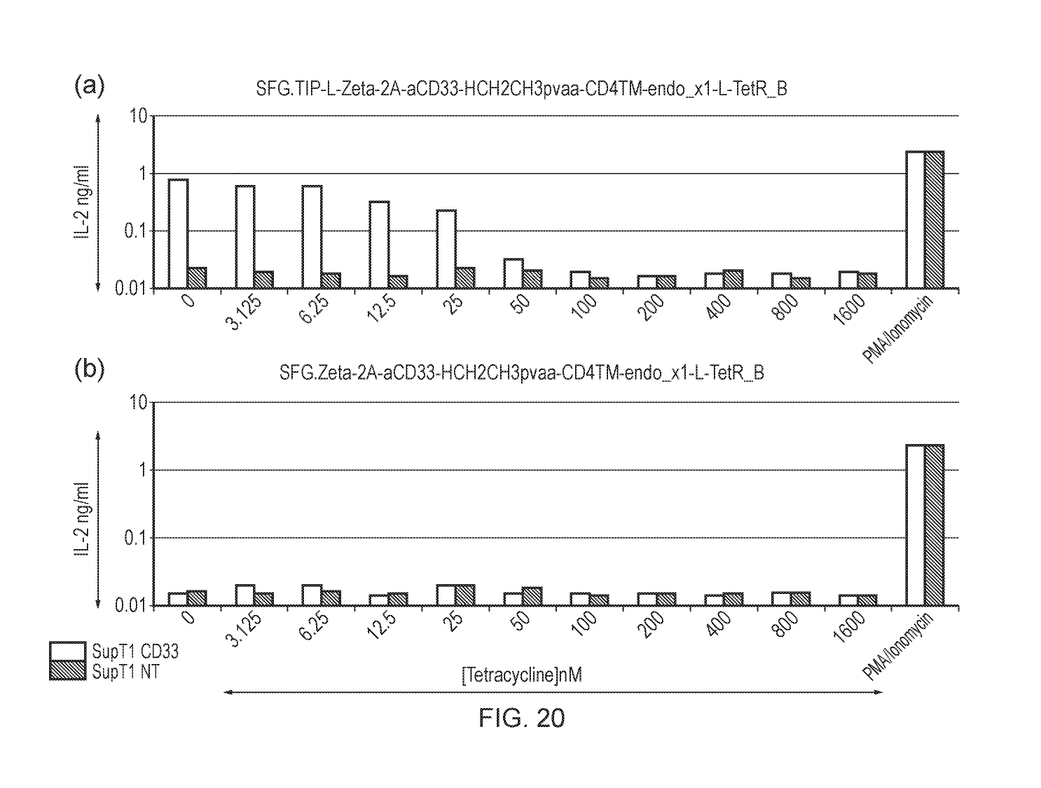

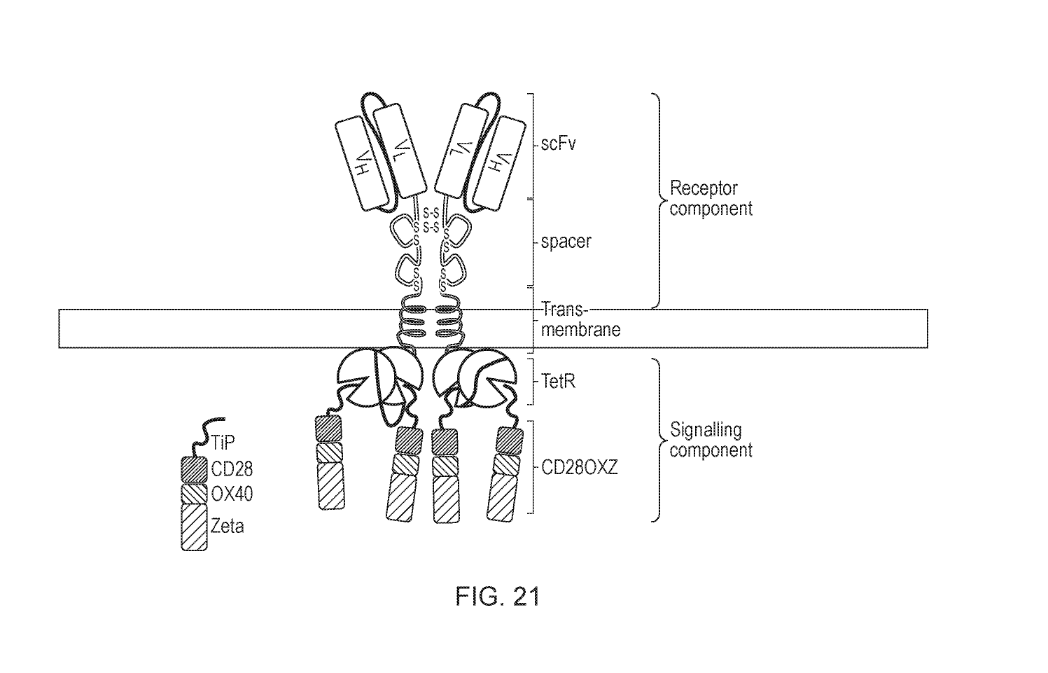

The BW5 T-cells were challenged with wild-type SupT1 cells or SupT1 cells engineered to express CD33 in the absence of tetracycline or in the presence of increasing concentrations of tetracycline. T-cells challenged with wild-type SupT1 cells did not activate in either the presence or absence of Tetracyline; T-cells challenged with SupT1 cells expressing CD33 were activated in the absence of Tetracycline, but activation is rapidly inhibited in the presence of tetracycline with activation fully inhibited in the presence of 100 nM of tetracycline (FIG. 20a).

Control TetCAR which lacks the TiP domain was also transduced into BW5. Once again, these T-cells were challenged with wild-type SupT1 cells or SupT1 cells engineered to express CD33 in the absence or in the presence of increasing concentration of Tetracycline. A lack of TiP element in signalling component resulted in no signalling in any conditions (FIG. 20b).

### Example 7—Signalling of a Tunable System in Primary T Cells

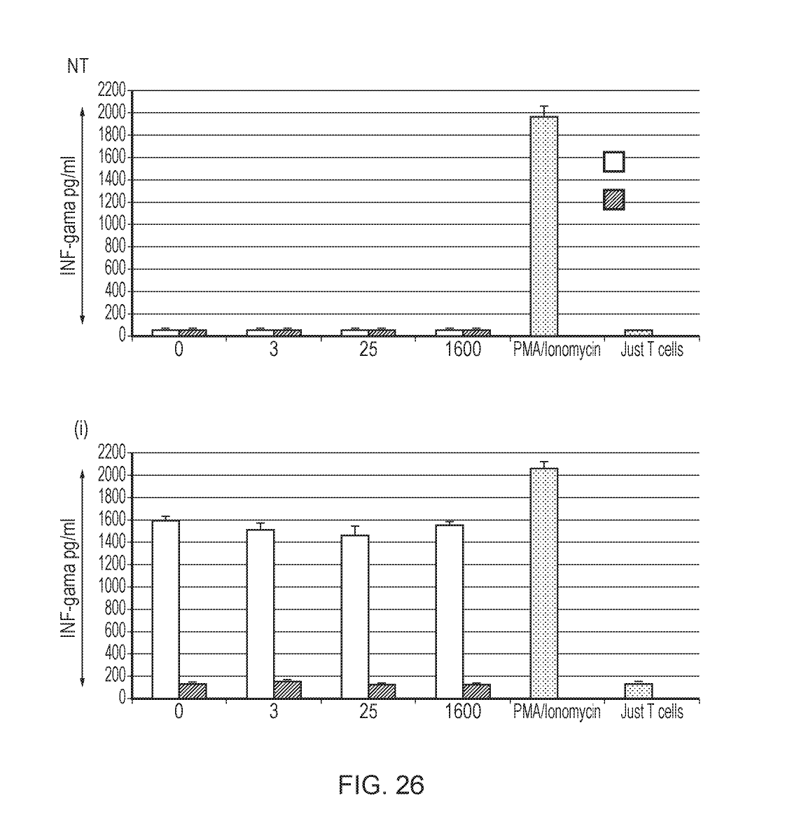

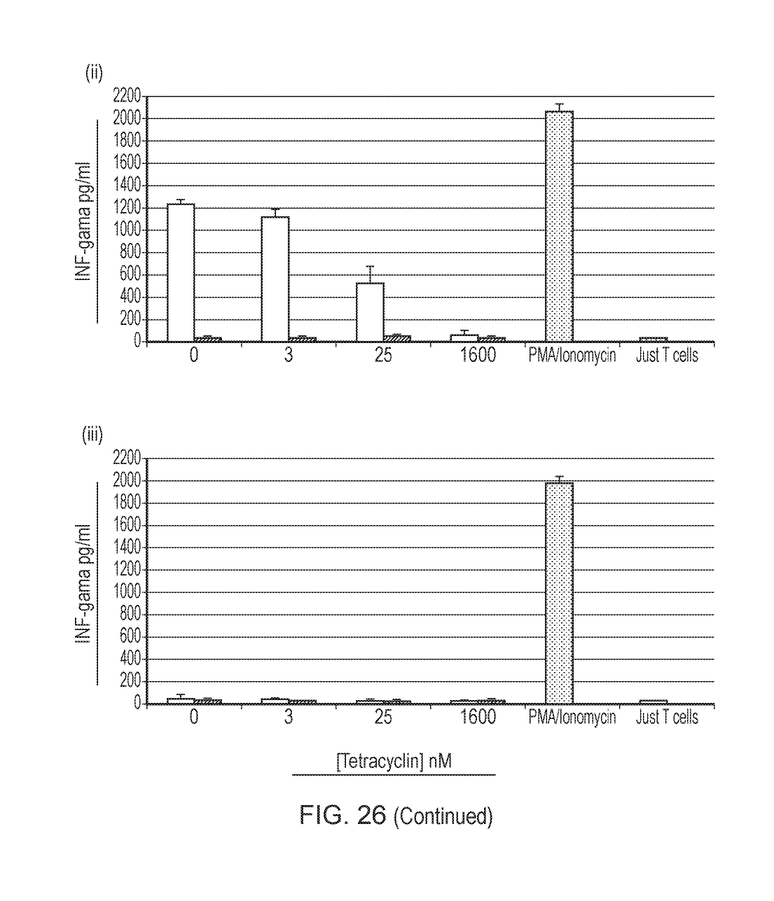

SupT1 cells (which are CD19 negative), were engineered to be CD19 positive giving target negative and positive cell lines which were as similar as possible. Primary human T-cells from 3 donors were transduced with three CAR constructs: (i) “Classical” 1st generation anti-CD19 CAR; (ii) 1st generation anti-CD19 tetCAR; (iii) Control anti-CD19 tetCAR where TiP is missing from endodomain. Non-transduced T-cells and T-cells transduced with the different CAR constructs were challenged 1:1 with either SupT1 cells or SupT1.CD19 cells in the presence of different concentrations of Tetracycline. Supernatant was sampled 48 hours after challenge. Supernatant from background (T-cells alone), and maximum (T-cells stimulated with PMA/lonomycin) was also samples. Interferon-gamma was measured in supernatants by ELISA (FIG. 26). “Classical” CAR T-cells were activated by SupT1.CD19 irrespective of tetracycline. TetCAR T-cell were activated by SupT1.CD19 cells but activation was inhibited by Tetracycline. The control TetCAR and NT T-cells did not respond to SupT1.CD19 cells.

### Example 8—Killing of Target Cells

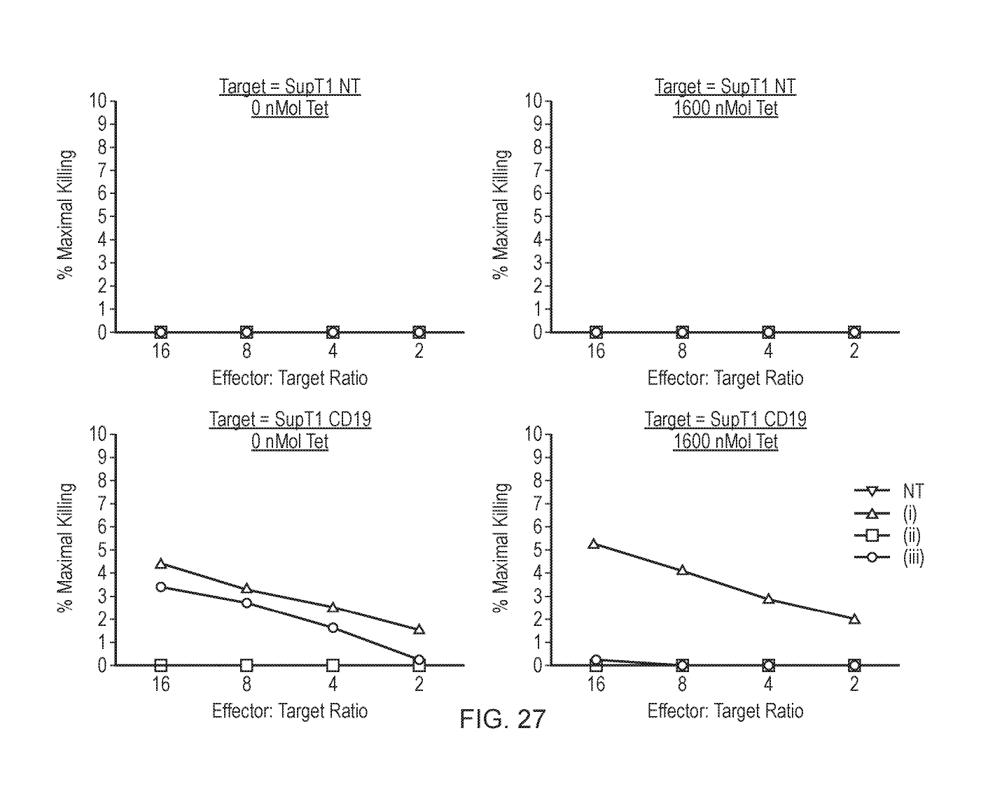

Following on from the interferon-gamma release study described in Example 7, killing of target cells was demonstrated using a chromium release assay. SupT1 and SupT1.CD19 cells were loaded with ⁵¹Cr and incubated with control and Tet-CAR T-cells for 4 hours in the presence or absence of tetracycline. Lysis of target cells was determined by counting ⁵¹Cr in the supernatant. The results are shown in FIG. 27. It was shown that Tet-CAR T-cells lysed SupT1.CD19 target cells only in the absence of Tetracycline.

### Example 9—Development of a Tunable CD19/CD22 OR Gate

The capacity of a variety of tunable CD19/CD22 OR gates to kill target cells is tested in the presence of varying concentrations of agent.

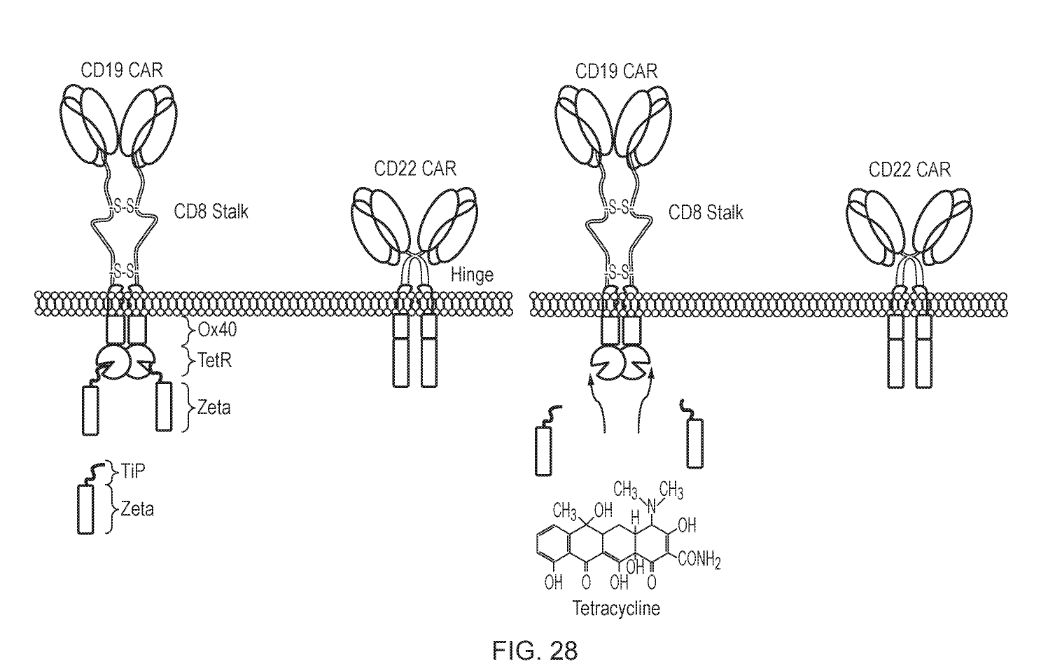

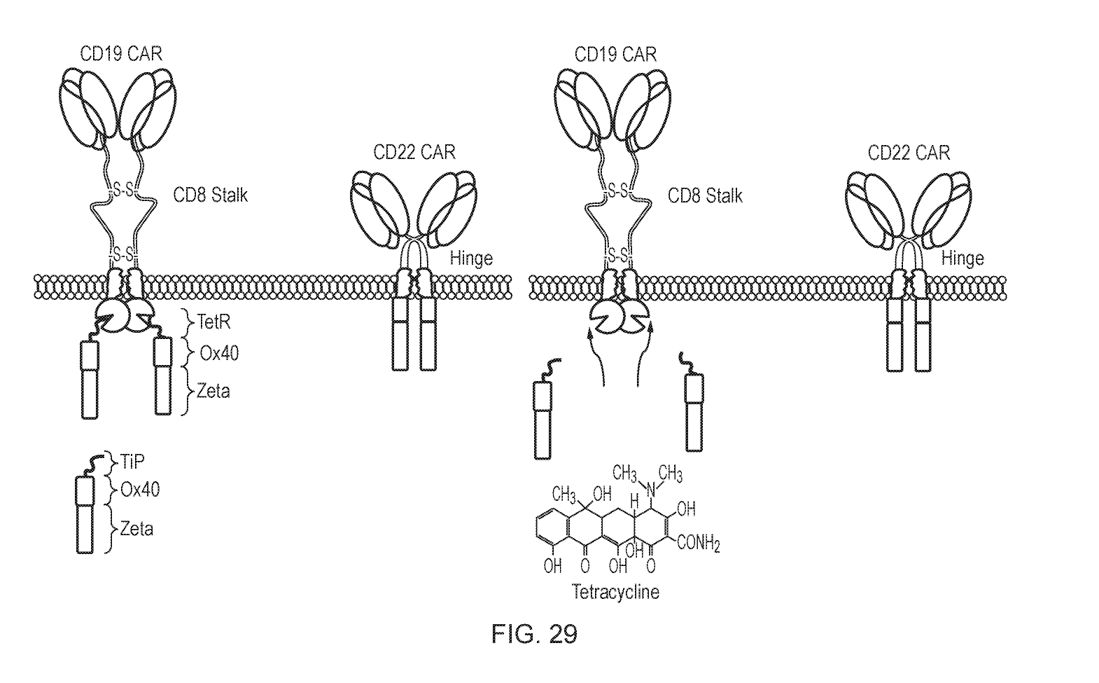

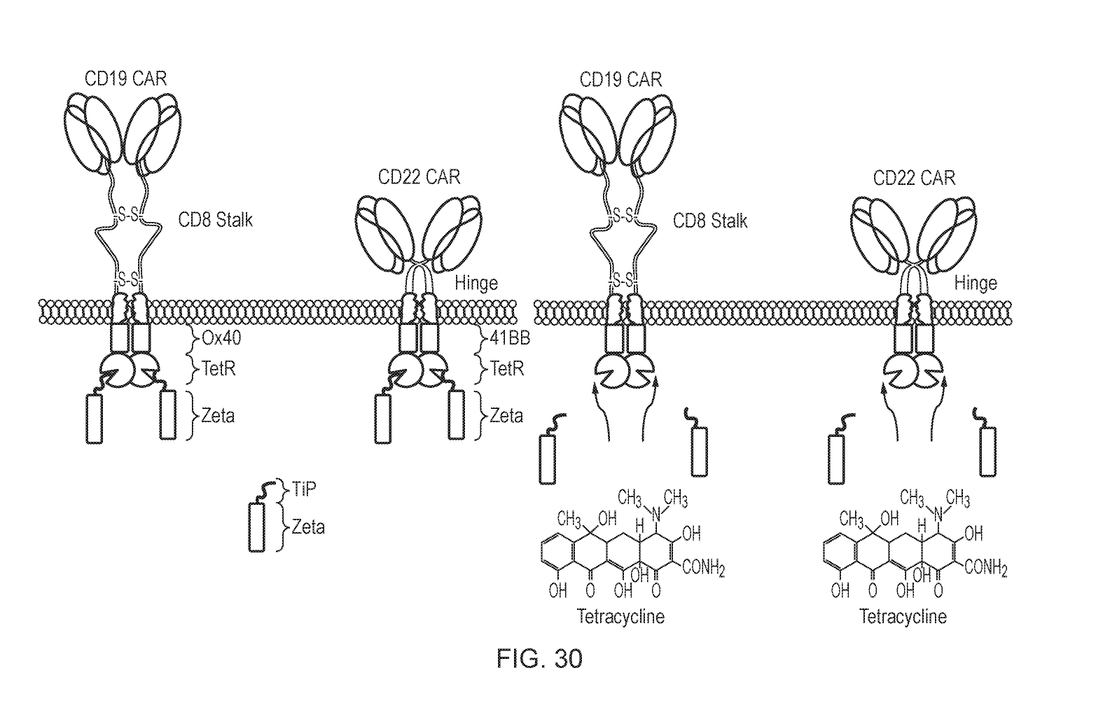

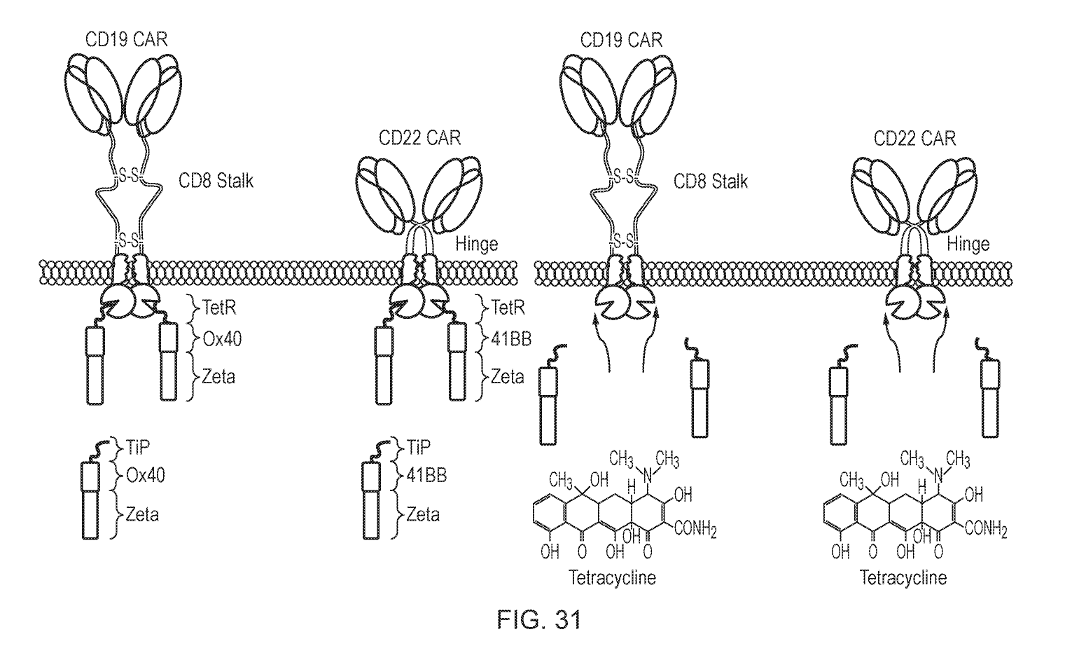

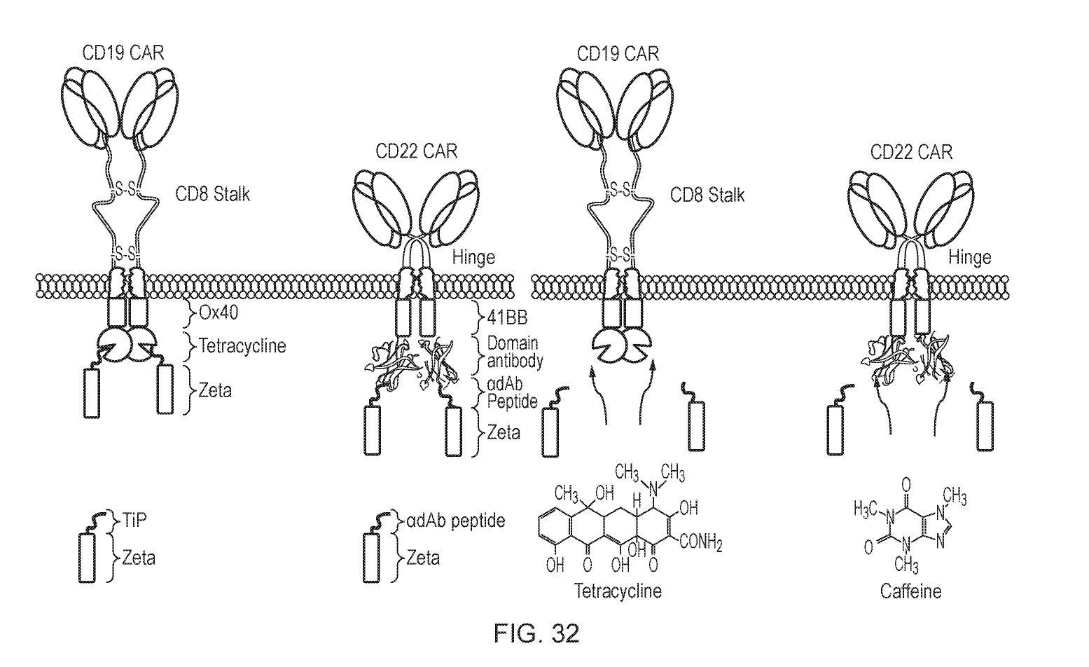

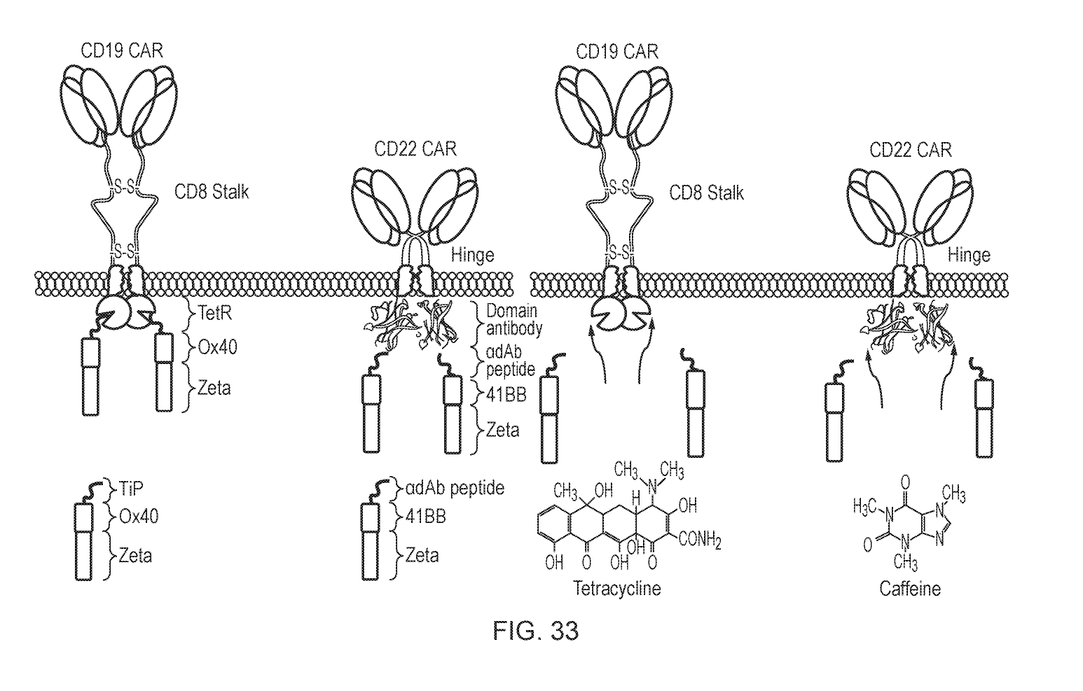

Multi-cistronic constructs expressing the CD19 CAR/CD22 CAR/intracellular signalling molecule(s) combinations illustrated in FIGS. 28 to 35. Retroviruses are produced by transient transfection of 293T cells with plasmids encoding the CARs, gag/pol and the envelope protein RD114. After 3 days the supernatants are harvested and used to transduce either PHA/IL2-activated PBMCs or BW5 T cells with equal titres of retrovirus on retronectin-coated plates. Six days post-transduction CAR-expression was confirmed by flow cytometry.

The transduced PBMCs/BW5 T cells are co-cultured in a 1:1 or 1:10 ratio with target cells, which may be:
NT SupT1 cells—expressing neither CD19 nor CD22
CD19+SupT1 cells—expressing CD19 only
CD22+SupT1 cells—expressing CD22 only
CD19+CD22+SupT1 cells—expressing CD19 and CD22
Raji cells—expressing CD19 and CD22
CD19−Raji cells—expressing CD22 only
CD22−Raji cells—expressing CD19 only

Target cell killing is assayed after one and three days. Also after one and three days, supernatants are removed and interferon-γ levels are assayed by ELISA.

The killing assays are conducted in the absence of agent or in the presence of increasing concentrations of agent. The agent may, for example be tetracycline, minocycline or caffeine.

Killing of target cells is demonstrated using a chromium release assay. Target cells are loaded with ⁵¹Cr and incubated with PBMC/T-cells for 4 hours in the presence or absence of agent. Lysis of target cells is determined by counting ⁵¹Cr in the supernatant.

### Example 10—A CD22ALAB-Based CAR with a Coiled-Coil Spacer Domain

A potential problem with OR gates occurs when one antigen is more amendable to target with a CAR-cased approach that the other antigen. This may be due to, for example, differences in antigen density between the two targets at the cell surface, or due to differences in the nature, such as the size, shape, charge or flexibility of the two antigens.

The human CD19 antigen has an extracellular domain of 271 amino acids. It has been successfully targeted using a CAR approach and is the subject of numerous clinical studies. The human CD22 antigen has an extracellular domain of 667 amino acids, arranged into 7 lg domains in a relatively linear, inflexible structure (FIG. 36A).

The present inventors have found that, when comparing anti-CD19 CARs with anti-CD22 CARs having an equivalent structure (same transmembrane domain and endodomains) and similar binding affinities, the CD19 CARs routinely out-perform the CD22-CARs in terms of killing of CD19+CD22+ target cells.

In order to investigate the effect of spacer type on anti-CD22 CAR function, an anti-CD22 CAR with a IgG1 hinge spacer was compared with an anti-CD22 CAR having the same antigen-binding domain but a coiled-coil spacer domain (FIG. 36B).

In more detail, a CAR having a CD22ALAb scFv antigen-binding domain, a COMP coiled-coil spacer domain, a transmembrane domain and an endodomain comprising 41BB and CD3zeta was made and compared with an equivalent CAR which comprises an IgG1 hinge spacer domain.

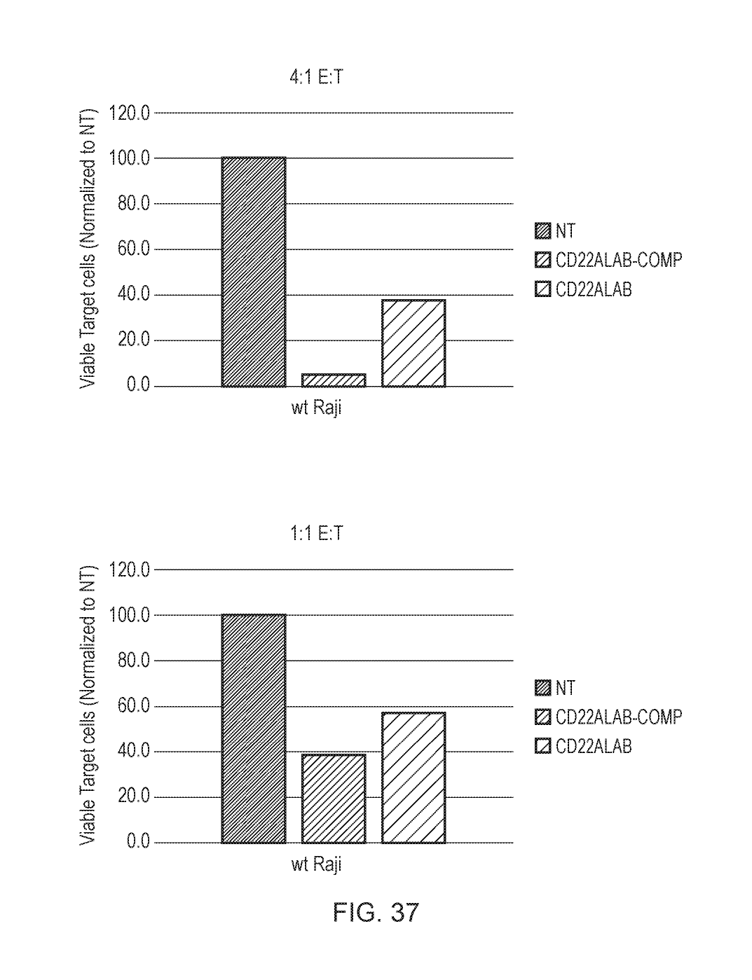

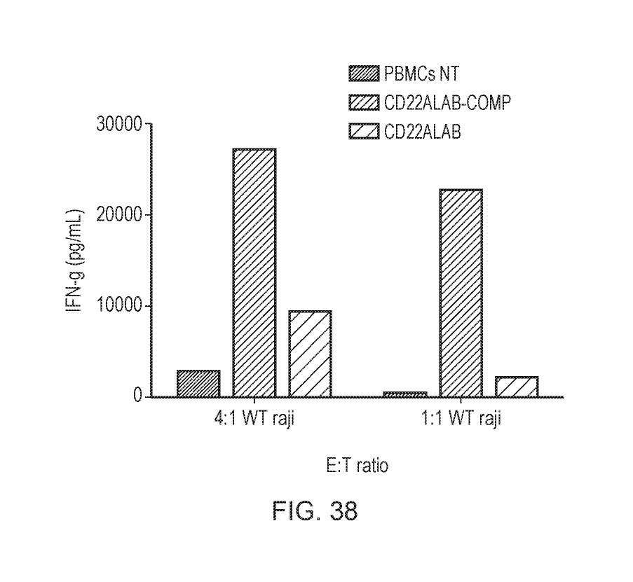

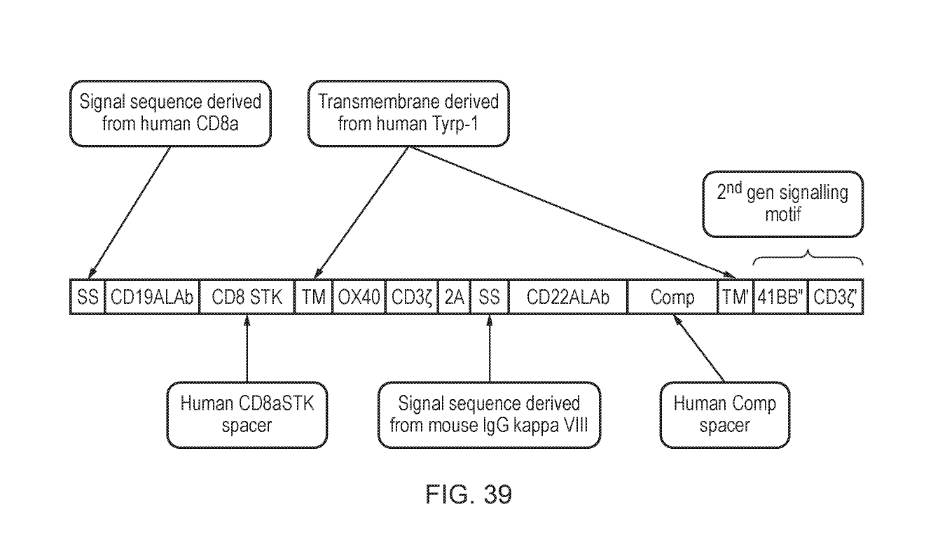

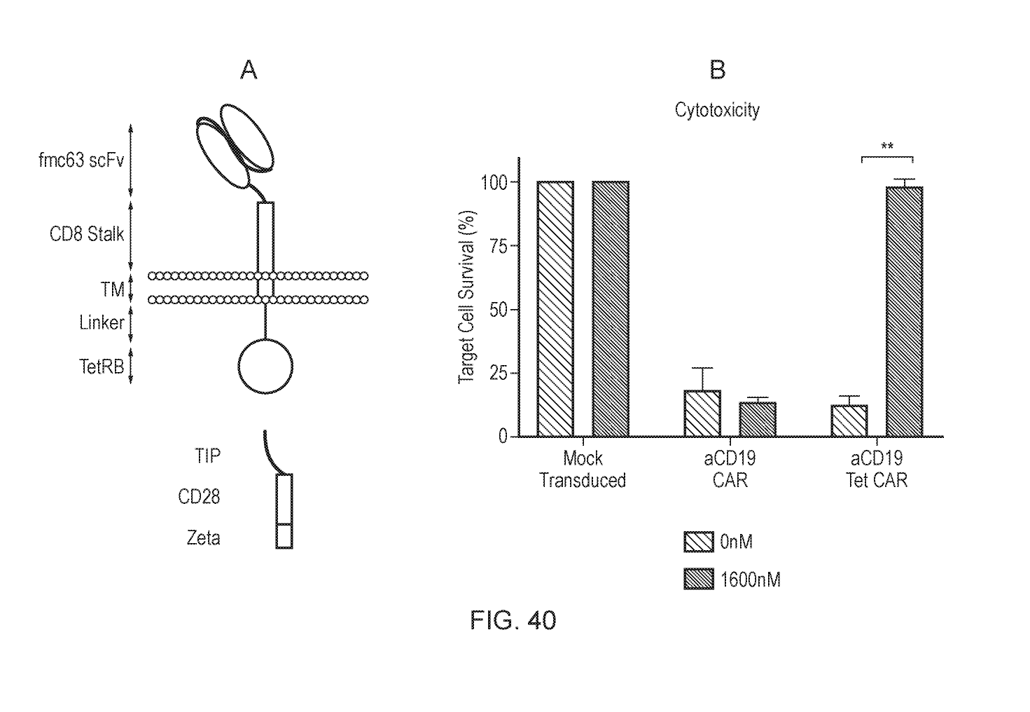

Retroviral vectors, as described above were used to transduce PBMCs from two separate donors with the CARs. The capacity of the cells to kill Raji cells, which are CD19 and CD22 positive, was determined at a 1:1 and a 4:1 E:T ratio. IFNγ secretion was also compared and the results are shown in FIGS. 37 and 38.

It was found that the anti-CD22 CAR with a coiled-coil spacer domain gave greater target cell killing and interferon gamma release than the equivalent CAR with an IgG1 hinge spacer domain.

### Example 11—Production of a Tunable Anti-CD19 CAR

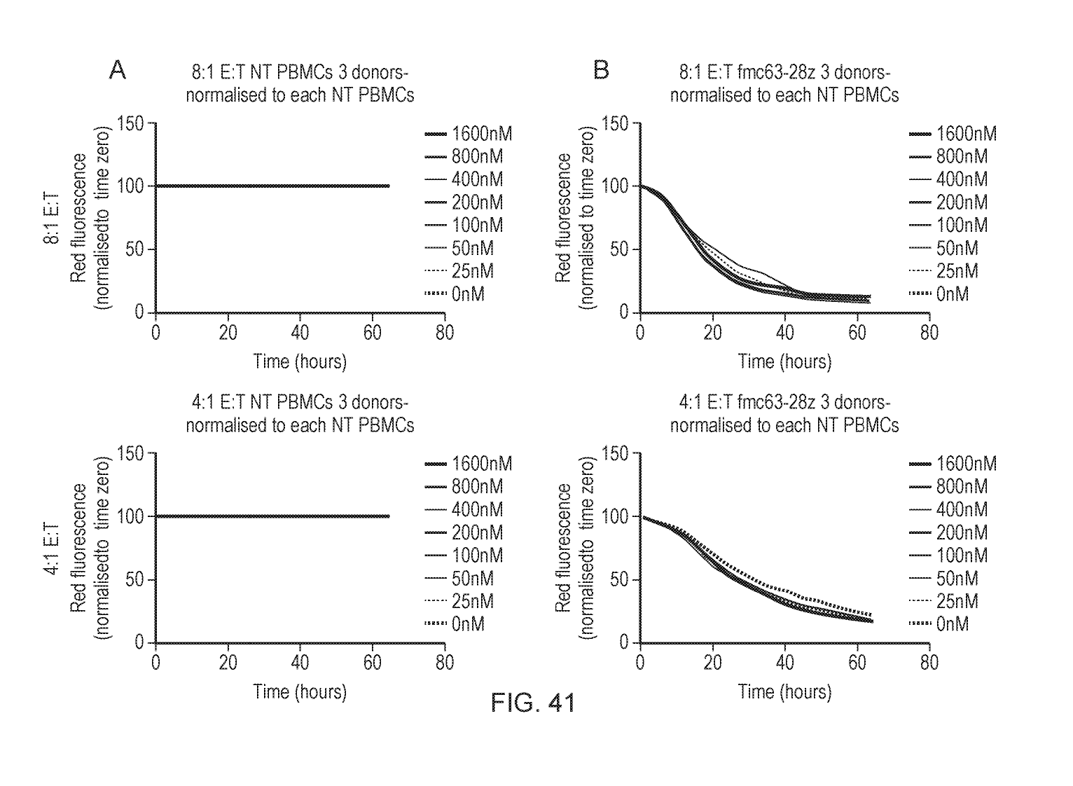

A bicistronic construct was expressed in T cells as a single transcript which self-cleaves at the 2A site to yield a signalling component which comprises TiP fused via a flexible linker to the endodomains of CD28 and CD3-Zeta; and a receptor component which comprises a CD19 recognizing scFv (Fmc63), a spacer and transmmbrane domain derived from CD8; and TetR (FIG. 40A).

PBMCs were either mock-transduced, transduced with a classical (non-tunable) CD19 CAR, or transduced with the tunable CD19 CAR.

Killing of target cells was demonstrated using an incucyte live-cell anaylsis system. CD19+ SKOV3 cells bearing a nuclear fluorescent protein were incubated with mock transduced, antiCD19 CAR-T cells (non-tunable) or anti-CD19 TetCAR T-cells at an 8:1 E:T ratio in the presence or absence of 1600 nM tetracycline. The machine was set up to take a picture of each well every hour. At the end of the experiment a “mask” was applied so that the software counts how many red cells are in each picture in each timepoint, in order to calculate the cytotoxicity. As shown in FIG. 40B, the control CAR eliminated the target cells regardless of the presence of tetracycline. However, the TetCAR eliminated the target cells only in the absence of tetracycline.

### Example 12—Demonstration of Tunable Cytotoxicity

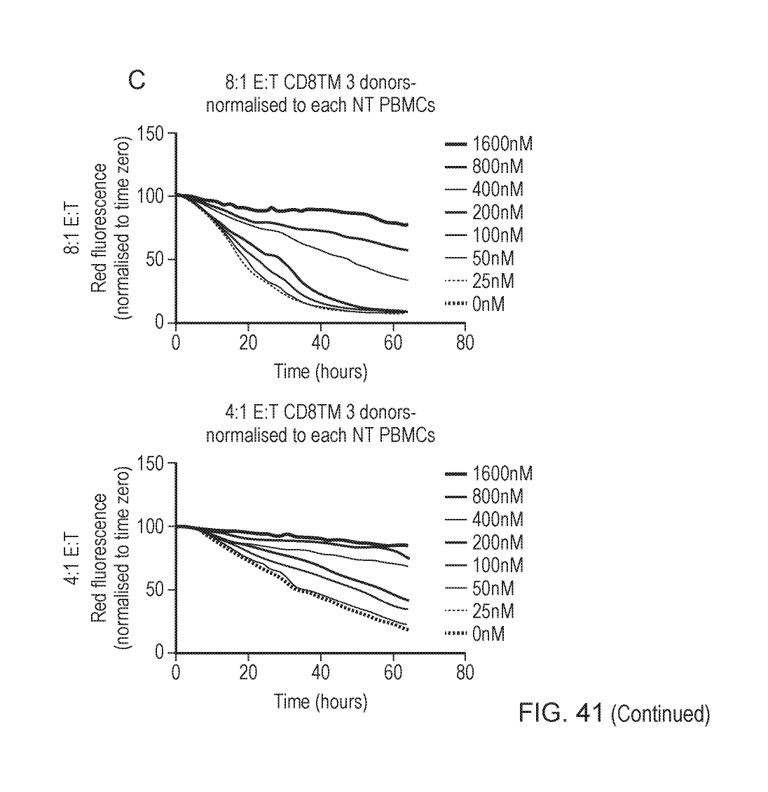

The assay described in Example 11 was repeated at an 8:1 or 4:1 E:T ratio with a range of concentrations of tetracycline (0 nM-1600 nM). An incucyte live-cell anaylsis system was used which measures killing over time. As shown in FIG. 41, the amount of target cell killing by the TetCAR is “tunable” depending on the concentration of tetracycline. The response to tetracycline exhibits an analogue, rather than digital, pattern.

### Example 13—Demonstration of Reversible Cytotoxicity

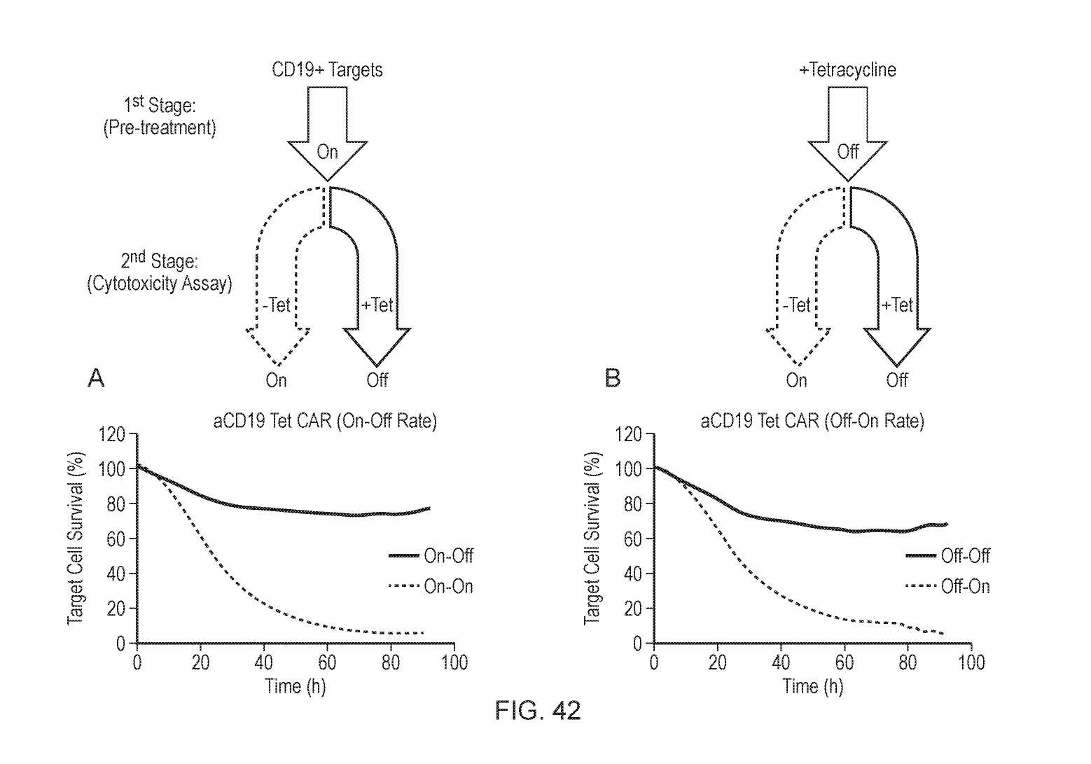

In order to investigate whether the effect of tetracycline is reversible, an “On-Off” and an “Off-On” study was conducted as illustrated in FIG. 42.

For the “On-Off” study (FIG. 42 A, shown in green) antiCD19 TetCAR T cells as described in Examples 11 and 12 were preincubated with CD19+ targets in the absence of tetracycline. After 2 hours, a cytotoxicity assay was conducted in the absence or presence of 1600 nM tetracycline. As shown in FIG. 42A, the capacity to kill target cells can be rapidly turned “off” by the addition of tetracycline.

For the “On-Off” study (FIG. 42 A, shown in green) antiCD19 TetCAR T cells as described in Examples 11 and 12 were preincubated with CD19+ targets in the presence of tetracycline. After 2 hours, a cytotoxicity was conducted in the absence or presence of 1600 nM tetracycline. As shown in FIG. 42B, the capacity to kill target cells can be rapidly turned “on” by the removal of tetracycline from the assay culture.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cell biology or related fields are intended to be within the scope of the following claims.

1. A cell which co-expresses a first chimeric antigen receptor (CAR) and second CAR at the cell surface, each CAR comprising an antigen-binding domain, a transmembrane domain and an intracellular domain
wherein the antigen-binding domain of the first CAR binds to CD19 and the antigen-binding domain of the second CAR binds to CD22; and
wherein the first and/or second CAR is a tunable CAR having an intracellular domain comprising a heterodimerization domain, which intracellular domain is capable of binding a separate intracellular signalling molecule which comprises a reciprocal heterodimerization domain and a signalling domain.

2. A cell according to claim 1, wherein binding of the first and second/or CAR to the intracellular signalling molecule is disrupted by the presence of an agent, such that in the absence of the agent the first and/or second CAR heterodimerize(s) with the intracellular signalling molecule and binding of the antigen binding domain to antigen results in signalling through the signalling domain; whereas in the presence of the agent, the first and/or second CAR do/does not heterodimerize with the intracellular signalling molecule and binding of the antigen binding domain to antigen does not result in signalling through the signalling domain.

3. A cell according to claim 1, wherein the first CAR, which binds to CD19, is a tunable CAR, having an intracellular domain which comprises a heterodimerization domain which binds a heterodimerization domain of an intracellular signalling molecule; and the second CAR, which binds to CD22, is a classical CAR, having an intracellular domain which comprises a signalling domain.

4-17. (canceled)

18. A nucleic acid construct encoding a first chimeric antigen receptor (CAR) and second CAR, each CAR comprising an antigen-binding domain, a transmembrane domain and an intracellular domain
wherein the antigen-binding domain of the first CAR binds to CD19 and the antigen-binding domain of the second CAR binds to CD22; and
wherein the first and/or second CAR is a tunable CAR having an intracellular domain comprising a heterodimerization domain, which intracellular domain is capable of binding a separate intracellular signalling molecule which comprises a reciprocal heterodimerization domain and a signalling domain.

19. A nucleic acid construct according to claim 18, which has one of the following structures:
a) AgB1-spacer1-TM1-HD1-coexpr-AgB2-spacer2-TM2-endo2;
b) AgB1-spacer1-TM1-endo1-coexpr-AgB2-spacer2-TM2-HD2;
c) AgB2-spacer2-TM2-HD2-coexpr-AgB1-spacer1-TM1-endo1;
d) AgB2-spacer2-TM2-endo2-coexpr-AgB1-spacer1-TM1-HD1;
e) AgB1-spacer1-TM1-HD1-coexpr-AgB2-spacer2-TM1-HD1;
f) AgB2-spacer2-TM2-HD2-coexpr-AgB1-spacer1-TM1-HD1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR
HD1 is a nucleic acid sequence encoding a heterodimerisation domain of the first CAR
Endo1 is a nucleic acid sequence encoding an intracellular domain which comprises a signalling domain of the first CAR;
coexpr is a nucleic acid sequence enabling co-expression of both CARs
AgB2 is a nucleic acid sequence encoding the antigen-binding domain of the second CAR;
spacer 2 is a nucleic acid sequence encoding the spacer of the second CAR;
TM2 is a a nucleic acid sequence encoding the transmembrane domain of the second CAR;
HD2 is a nucleic acid sequence encoding a heterodimerisation domain of the second CAR
Endo2 is a nucleic acid sequence encoding an intracellular domain which comprises a signalling domain of the second CAR.

20-21. (canceled)

22. A nucleic acid construct according to claim 18, which also comprises a nucleic acid sequence encoding an intracellular signalling molecule which comprises a heterodimerization domain reciprocal to the heterodimerization domain on the tunable CAR, and a signalling domain.

23. A kit which comprises
a first nucleic acid sequence encoding a first chimeric antigen receptor (CAR) and second nucleic acid sequence encoding a second CAR, each CAR comprising an antigen-binding domain, a transmembrane domain and an intracellular domain
wherein the antigen-binding domain of the first CAR binds to CD19 and the antigen-binding domain of the second CAR binds to CD22; and
wherein the first and/or second CAR is a tunable CAR having an intracellular domain comprising a heterodimerization domain, which intracellular domain is capable of binding a separate intracellular signalling molecule which comprises a reciprocal heterodimerization domain and a signalling domain wherein
(i) the first nucleic acid sequence has the following structures:
AgB1-spacer1-TM1-HD1 or AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
HD1 is a nucleic acid sequence encoding a heterodimerisation domain of the first CAR; and
Endo1 is a nucleic acid sequence encoding an intracellular domain which comprises a signalling domain of the first CAR
and
(ii) the second nucleic acid sequence has the following structure:
AgB2-spacer2-TM2-HD2 or AgB2-spacer2-TM2-endo2
in which
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
HD2 is a nucleic acid sequence encoding a heterodimerisation domain of the second CAR; and
Endo2 is a nucleic acid sequence encoding an intracellular domain which comprises a signalling domain of the second CAR.

24. A kit according to claim 23, which also comprises (iii) a third nucleic acid sequence encoding an intracellular signalling molecule which comprises a heterodimerization domain reciprocal to the heterodimerization domain on the tunable CAR, and a signalling domain.

25. A kit comprising: a first vector which comprises a first nucleic acid sequence encoding a first chimeric antigen receptor (CAR); and a second vector which comprises a second nucleic acid sequence encoding a second CAR, each CAR comprising an antigen-binding domain, a transmembrane domain and an intracellular domain
wherein the antigen-binding domain of the first CAR binds to CD19 and the antigen-binding domain of the second CAR binds to CD22; and
wherein the first and/or second CAR is a tunable CAR having an intracellular domain comprising a heterodimerization domain, which intracellular domain is capable of binding a separate intracellular signalling molecule which comprises a reciprocal heterodimerization domain and a signalling domain.

26. A vector comprising a nucleic acid construct according to claim 18.

27. (canceled)

28. A method for making a cell according to claim 1, which comprises the step of introducing: a nucleic acid construct according to claim 18 into a cell.

29. (canceled)

30. A pharmaceutical composition comprising a plurality of cells according to claim 1.

31. A method for treating and/or preventing a disease, which comprises the step of administering a pharmaceutical composition according to claim 30 to a subject.

32. A method according to claim 31, which comprises the following steps:
(i) isolation of a cell-containing sample from a subject;
(ii) transduction or transfection of the cells with: a nucleic acid construct according to claim 18; and
(iii) administering the cells from (ii) to a the subject.

33. A method according to claim 31, which involves monitoring toxic activity in the subject and comprises the step of administering an agent to the subject to reduce adverse toxic effects.

34. A method according to claim 31, wherein the disease is a cancer.

35. A method according to claim 34, wherein the cancer is a B cell malignancy.

36-37. (canceled)

38. A method for inhibiting a tunable CAR system of a cell according claim 2, which comprises the step of administering an agent which disrupts binding of the first and second/or CAR to the intracellular signalling molecule.

39. A kit according to claim 25 which comprises a third vector comprising a third nucleic acid sequence encoding an intracellular signalling molecule which comprises a heterodimerization domain reciprocal to the heterodimerization domain on the tunable CAR, and a signalling domain.


SEQUENCE LISTING (71 sequences)

SEQ ID NO: 1 - protein, 5 aa, Artificial Sequence
  heavy chain variable region (VH) complementarity determining region (CDR) CDR1
SYWMN

SEQ ID NO: 2 - protein, 16 aa, Artificial Sequence
  heavy chain variable region (VH) CDR2
QIWPGDGDTNYNGKFK

SEQ ID NO: 3 - protein, 15 aa, Artificial Sequence
  heavy chain variable region (VH) CDR3
RETTTVGRYYYAMDY

SEQ ID NO: 4 - protein, 15 aa, Artificial Sequence
  light chain variable region (VL) CDR1
KASQSVDYDGDSYLN

SEQ ID NO: 5 - protein, 7 aa, Artificial Sequence
  light chain variable region (VL) CDR2
DASNLVS

SEQ ID NO: 6 - protein, 9 aa, Artificial Sequence
  light chain variable region (VL) CDR3
QQSTEDPWT

SEQ ID NO: 7 - protein, 124 aa, Artificial Sequence
  chimeric antigen receptor (CAR), Murine CD19ALAb VH sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 8 - protein, 124 aa, Artificial Sequence
  CAR, Humanised CD19ALAb VH sequence
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 9 - protein, 111 aa, Artificial Sequence
  CAR, Murine CD19ALAb VL sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 10 - protein, 112 aa, Artificial Sequence
  CAR, Humanised CD19ALAb VL sequence, Kappa 16
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 11 - protein, 112 aa, Artificial Sequence
  CAR, Humanised CD19ALAb VL sequence, Kappa 7
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 12 - protein, 235 aa, Artificial Sequence
  CAR, Murine CD19ALAb scFv sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 13 - protein, 236 aa, Artificial Sequence
  CAR, Humanised CD19ALAb scFv sequence - Heavy 19, Kappa 16
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 14 - protein, 236 aa, Artificial Sequence
  CAR, Humanised CD19ALAb scFv sequence - Heavy 19, Kappa 7
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 15 - protein, 5 aa, Artificial Sequence
  heavy chain variable region (VH) complementarity determining region (CDR) CDR1
NYWIN

SEQ ID NO: 16 - protein, 17 aa, Artificial Sequence
  heavy chain variable region (VH) CDR2
NIYPSDSFTNYNQKFKD

SEQ ID NO: 17 - protein, 11 aa, Artificial Sequence
  heavy chain variable region (VH) CDR3
DTQERSWYFDV

SEQ ID NO: 18 - protein, 16 aa, Artificial Sequence
  light chain variable region (VL) CDR1
RSSQSLVHSNGNTYLH

SEQ ID NO: 19 - protein, 7 aa, Artificial Sequence
  light chain variable region (VL) CDR2
KVSNRFS

SEQ ID NO: 20 - protein, 9 aa, Artificial Sequence
  light chain variable region (VL) CDR3
SQSTHVPWT

SEQ ID NO: 21 - protein, 120 aa, Artificial Sequence
  CAR, Murine CD22ALAb VH sequence
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSS

SEQ ID NO: 22 - protein, 120 aa, Artificial Sequence
  CAR, Humanised CD22ALAb VH sequence
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSS

SEQ ID NO: 23 - protein, 112 aa, Artificial Sequence
  CAR, Murine CD22ALAb VL sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 24 - protein, 112 aa, Artificial Sequence
  CAR, Humanised CD22ALAb VL sequence
DIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 25 - protein, 232 aa, Artificial Sequence
  CAR, Murine CD22ALAb scFv sequence
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSSDVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 26 - protein, 232 aa, Artificial Sequence
  CAR, Humanised CD22ALAb scFv sequence
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSSDIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 27 - protein, 139 aa, Artificial Sequence
  TetR binding domain
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGH

SEQ ID NO: 28 - protein, 17 aa, Artificial Sequence
  TiP binding domain
MWTWNAYAFAAPSGGGS

SEQ ID NO: 29 - protein, 61 aa, Artificial Sequence
  modified CD4 endodomain linker
ALIVLGGVAGLLLFIGLGIFFCVRCRHRRRQAERMAQIKRVVSEKKTAQAPHRFQKTCSPI

SEQ ID NO: 30 - protein, 15 aa, Artificial Sequence
  Biotin mimicking peptide, long nanotag
DVEAWLDERVPLVET

SEQ ID NO: 31 - protein, 9 aa, Artificial Sequence
  Biotin mimicking peptide, short nanotag
DVEAWLGAR

SEQ ID NO: 32 - protein, 8 aa, Artificial Sequence
  Biotin mimicking peptide, streptag
WRHPQFGG

SEQ ID NO: 33 - protein, 8 aa, Artificial Sequence
  Biotin mimicking peptide, streptagII
WSHPQFEK

SEQ ID NO: 34 - protein, 38 aa, Artificial Sequence
  Biotin mimicking peptide, SBP-tag
MDEKTTGWRGGHVVEGLAGELEQLRARLEHHPQGQREP

SEQ ID NO: 35 - protein, 8 aa, Artificial Sequence
  Biotin mimicking peptide, ccstreptag
CHPQGPPC

SEQ ID NO: 36 - protein, 15 aa, Artificial Sequence
  Biotin mimicking peptide, flankedccstreptag
AECHPQGPPCIEGRK

SEQ ID NO: 37 - protein, 126 aa, Artificial Sequence
  core streptavidin sequence
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 38 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 39 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 40 - protein, 20 aa, Artificial Sequence
  spacer sequence, human IgG1 hinge
AEPKSPDKTHTCPPCPKDPK

SEQ ID NO: 41 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 42 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 43 - protein, 24 aa, Artificial Sequence
  tyrp-1 transmembrane sequence
IIAIAVVGALLLVALIFGTASYLI

SEQ ID NO: 44 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 140 aa, Artificial Sequence
  CD28 transmembrane domain and CD3 Z endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 180 aa, Artificial Sequence
  CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 216 aa, Artificial Sequence
  CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 20 aa, Thosea asigna virus
RAEGRGSLLTCGDVEENPGP

SEQ ID NO: 49 - protein, 1133 aa, Artificial Sequence
  CD19/CD22 ′OR′ gate construct
MSLPVTALLLPLALLLHAARPYPYDVPDYASLSGGGGSQVQLVQSGAEVKKPGASVKVSCKASGYTFTSNWMHWVRQAPGQGLEWMGEIDPSDSYTNYNQKFKGRVTITVDKSASTAYMELSSLRSEDTAVYYCARGSNPYYYAMDYWGQGTLVTVSSGGGGSGGGGSGGGGSEIVLTQSPATLSLSPGERATLSCSASSGVNYMHWYQQKPGQAPRRWIYDTSKLASGVPARFSGSGSGTSYSLTISSLEPEDFAVYYCHQRGSYTFGGGTKLEIKRSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 57 aa, Artificial Sequence
  sequence of coiled coil domain (Kinesin motor protein parallel homodimer)
MHAALSTEVVHLRQRTEELLRCNEQQAAELETCKEQLFQSNMERKELHNTVMDLRGN

SEQ ID NO: 51 - protein, 50 aa, Artificial Sequence [sequence omitted]

SEQ ID NO: 52 - protein, 61 aa, Artificial Sequence
  sequence of coiled coil domain (Archaeal box C/D sRNP core protein anti-parallel heterodimer)
RYVVALVKALEEIDESINMLNEKLEDIRAVKESEITEKFEKKIRELRELRRDVEREIEEVM

SEQ ID NO: 53 - protein, 31 aa, Artificial Sequence
  sequence of coiled coil domain (Mannose-binding protein A parallel homotrimer)
AIEVKLANMEAEINTLKSKLELTNKLHAFSM

SEQ ID NO: 54 - protein, 29 aa, Artificial Sequence
  sequence of coiled coil domain (Coiled-coil serine-rich protein 1 parallel homotrimer)
EWEALEKKLAALESKLQALEKKLEALEHG

SEQ ID NO: 55 - protein, 24 aa, Artificial Sequence
  sequence of coiled coil domain (Polypeptide release factor 2 anti-parallel heterotrimer Chain A)
INPVNNRIQDLTERSDVLRGYLDY

SEQ ID NO: 56 - protein, 51 aa, Artificial Sequence
  sequence of coiled coil domain (Polypeptide release factor 2 anti-parallel heterotrimer Chain B)
VVDTLDQMKQGLEDVSGLLELAVEADDEETFNEAVAELDALEEKLAQLEFR

SEQ ID NO: 57 - protein, 51 aa, Artificial Sequence
  sequence of coiled coil domain (SNAP-25 and SNARE parallel heterotetramer Chain A)
IETRHSEIIKLENSIRELHDMFMDMAMLVESQGEMIDRIEYNVEHAVDYVE

SEQ ID NO: 58 - protein, 67 aa, Artificial Sequence
  sequence of coiled coil domain (SNAP-25 and SNARE parallel heterotetramer Chain B)
ALSEIETRHSEIIKLENSIRELHDMFMDMAMLVESQGEMIDRIEYNVEHAVDYVERAVSDTKKAVKY

SEQ ID NO: 59 - protein, 69 aa, Artificial Sequence
  sequence of coiled coil domain (SNAP-25 and SNARE parallel heterotetramer Chain C)
ELEEMQRRADQLADESLESTRRMLQLVEESKDAGIRTLVMLDEQGEQLERIEEGMDQINKDMKEAEKNL

SEQ ID NO: 60 - protein, 51 aa, Artificial Sequence; identical to SEQ ID NO: 57
  sequence of coiled coil domain (SNAP-25 and SNARE parallel heterotetramer Chain D)

SEQ ID NO: 61 - protein, 20 aa, Artificial Sequence
  sequence of coiled coil domain (Lac repressor parallel homotetramer)
SPRALADSLMQLARQVSRLE

SEQ ID NO: 62 - protein, 136 aa, Artificial Sequence
  sequence of coiled coil domain (Apolipoprotein E anti-parallel heterotetramer)
SGQRWELALGRFWDYLRWVQTLSEQVQEELLSSQVTQELRALMDETMKELKAYKSELEEQLTARLSKELQAAQARLGADMEDVCGRLVQYRGEVQAMLGQSTEELRVRLASHLRKLRKRLLRDADDLQKRLAVYQA

SEQ ID NO: 63 - protein, 45 aa, Artificial Sequence
  COMP coiled-coil domain
DLGPQMLRELQETNAALQDVRELLRQQVREITFLKNTVMECDACG

SEQ ID NO: 64 - protein, 5 aa, Artificial Sequence
  truncated COMP coiled-coil domain (5 C-terminal amino acids)
CDACG

SEQ ID NO: 65 - protein, 20 aa, Artificial Sequence
  truncated COMP coiled-coil domain (C-terminus)
QQVREITFLKNTVMECDACG

SEQ ID NO: 66 - DNA, 1139 bp, Homo sapiens
atcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgcaga

SEQ ID NO: 67 - DNA, 1139 bp, Artificial Sequence
  codon-wobbled HCH2CH3-CD28tmZeta
atccagccgaaccaaagagccccgataagacccacacctgtcccccctgcccagccccagagctgctgggaggccccagcgtgtttctgtttccacccaagccaaaggataccctgatgattagtagaacacccgaagtgacctgtgtggtggtggatgtgtctcacgaggaccccgaggtgaaatttaattggtatgttgatggtgttgaagtgcacaacgccaaaaccaaacccagagaggagcagtacaattctacctatagagtcgtgtctgtgctgacagtgctgcatcaggattggctgaacggaaaagaatacaaatgtaaagtgagcaataaggccctgcccgctccaattgagaagacaattagcaaggccaagggccagccaagggagccccaggtgtatacactgccacccagtagagacgaactgacaaagaatcaggtgtctctgacatgtctggtgaagggattttacccatctgatatcgccgtggaatgggaatctaacggccagcccgagaataactataagacaaccccaccagtgctggatagcgatggcagcttttttctgtattctaagctgacagtggataagtcccggtggcagcagggaaatgtgtttagctgtagtgtcatgcatgaggccctgcacaatcactatacccagaaatctctgagtctgagcccaggcaagaaggaccccaagttctgggtcctggtggtggtgggaggcgtgctggcctgttactctctcctggtgaccgtggccttcatcatcttttgggtgcgctcccgggtgaagttttctcgctctgccgatgccccagcctatcagcagggccagaatcagctgtacaatgaactgaacctgggcaggcgggaggagtacgacgtgctggataagcggagaggcagagaccccgagatgggcggcaaaccacggcgcaaaaatccccaggagggactctataacgagctgcagaaggacaaaatggccgaggcctattccgagatcggcatgaagggagagagaagacgcggaaagggccacgacggcctgtatcagggattgtccaccgctacaaaagatacatatgatgccctgcacatgcaggccctgccacccaga

SEQ ID NO: 68 - protein, 258 aa, Homo sapiens
EEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARP

SEQ ID NO: 69 - protein, 16 aa, Artificial Sequence
  sequence of TiP
WTWNAYAFAAPSGGGS

SEQ ID NO: 70 - protein, 87 aa, Homo sapiens
LSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 71 - protein, 926 aa, Artificial Sequence
  basic TetCAR sequence
MWTWNAYAFAAPSGGGSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPMALIVLGGVAGLLLFIGLGIFFCVRCRHRRRQAERMAQIKRVVSEKKTAQAPHRFQKTCSPISGGGGSMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS